(19) United States
(12) Patent Application Publication
(10) Pub. No.: US 2024/0424112 A1
(43) Pub. Date: Dec. 26, 2024

(54) TETRAHYDROISOQUINOLINE COMPOUNDS THAT ARE KEAP1 BINDERS.

(71) Applicant: C4X Discovery Limited, Manchester (GB)

(72) Inventors: Catherine Louise Lucas, Manchester (GB); Nicholas Charles Ray, London (GB); William Rameshchandra Krishna Esmieu, Saffron Walden (GB)

(21) Appl. No.: 18/703,919

(22) PCT Filed: Oct. 25, 2022
(86) PCT No.: PCT/GB22/52719

§ 371 Date: Apr. 23, 2024

(30) Foreign Application Priority Data
Oct. 25, 2021 (GB) 2115330.9
Oct. 25, 2021 (GB) 2115334.1

(51) Int. Cl. A61K 47/55; A61K 47/54

(52) U.S. Cl. CPC A61K 47/55; A61K 47/542

(57) ABSTRACT

The present invention relates to tetrahydroisoquinoline compounds that are KEAP1 binders and their incorporation in bifunctional compounds which are capable of acting as proteolysis-targeting chimeras. The present invention also relates to processes for the preparation of these bifunctional compounds, and to their use in the treatment of diseases or disorders associated with proteins that may be degraded through the KEAP1 E3 ligase.

### INTRODUCTION

The present invention relates to tetrahydroisoquinoline compounds that are KEAP1 binders and their incorporation in bifunctional compounds which are capable of acting as protein degraders. The present invention also relates to processes for the preparation of these bifunctional compounds, and to their use in the treatment of diseases or disorders associated with proteins that may be degraded through the KEAP1 E3 ligase.

### BACKGROUND OF THE INVENTION

PROTAC®s (proteolysis-targeting chimeras) are protein degrading compounds that consist of an E3 ligase recruiter, a ligand to target the protein of interest (POI), and a linker to attach these two functionalities to one another. Through binding of the E3 ligase recruiter to its ligase, the PROTAC® is able to facilitate ubiquitination of the POI, leading to proteasomal degradation of the POI. To date only a small number of E3 ligase recruiters have been identified for the approximately 600 predicted human family E3 ligases, the four most widely used being the thalidomide-type immune-modulatory drugs that recruit cereblon (CRBN); hydroxyproline ligands that target the von-Hippel Lindau (VHL) E3 ligase; nutlins that recruit MDM2 and ligands that target cIAP. The absence of a wider range of E3 ligase-targeting functionality restricts the range of POIs that can be degraded, hence there is a need to identify effective ligase-recruiting groups for a broader range of E3 ligases. Kelch-like ECH-associated protein 1 (KEAP1) is a Bric-a-Brac (BTB)-Kelch protein that functions as a substrate adaptor protein for a Cullin 3 (CUL3)/Ring-Box 1 (RBX1)-dependent E3 ubiquitin ligase complex (doi: 10.1128/MCB.24.16.7130-7139.2004) that has as its most well-known substrate the nuclear transcription factor Nrf2. The KEAP1-CUL3 E3 ligase complex binds to Nrf2 through its N-terminal Neh2 domain and promotes 26S ubiquitin proteasomal degradation. KEAP1 has also been shown to be involved in the degradation of other substrates such as IKKb (doi:10.1016/j.molcel.2009.07.025). It is therefore anticipated that other POIs can be targeted for degradation through recruitment of KEAP1 and its associated complex by means of a suitable KEAP1 binding motif attached to a ligand selectively binding the POI. Such an approach has recently been validated for the BET family bromodomain POI with the known KEAP1 ligand bardoxolone (doi: 10.1038/s41598-020-72491-9) and a second ligand KI-696 (doi: 10.1021/jacs.1c04841) bound to the BET inhibitor JQ-1. WO2020/018788 describes benzotriazole degraders that target proteins via the KEAP1 ligase. WO2020/210229 describes bifunctional molecules that degrade KEAP1 (i.e. KEAP1 is the POI). The scope of KEAP1 E3 ligase-based PROTAC®s has recently been examined (doi: 10.1016/j.chembiol.2022.08.003).

There is therefore a need for the identification of bifunctional protein degraders (PROTAC®s) bearing selective KEAP1 ligand recruiting groups.

### SUMMARY OF THE INVENTION

In one aspect, the present invention provides novel bifunctional compounds which function to recruit targeted proteins to the KEAP1 E3 ligase complex for degradation.

In one aspect, the present invention provides a compound of Formula I, or a pharmaceutically acceptable salt, thereof as defined herein.

In another aspect, the present invention provides a pharmaceutical composition comprising a compound of Formula I as defined herein, or a pharmaceutically acceptable salt thereof, and one or more pharmaceutically acceptable excipients.

In another aspect, the present invention relates to a compound of Formula I as defined herein, or a pharmaceutically acceptable salt thereof, or a pharmaceutical composition as defined herein, for use in therapy.

In another aspect, the present invention relates to a method of treating a disease or disorder mediated by degradation of a protein of interest, said method comprising administering to a subject in need of such treatment a therapeutically effective amount of a compound of Formula I as defined herein, or a pharmaceutically acceptable salt thereof, or a pharmaceutical composition as defined herein.

The present invention further provides a method of synthesising a compound of Formula I, or a pharmaceutically acceptable salt thereof, as defined herein.

In another aspect, the present invention provides a compound of Formula I, or a pharmaceutically acceptable salt thereof, obtainable by, or obtained by, or directly obtained by a method of synthesis as defined herein.

Preferred, suitable, and optional features of any one particular aspect of the present invention are also preferred, suitable, and optional features of any other aspect.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

As used herein the term ‘KBG’ refers to a KEAP1 Binding Group.

As used herein the term ‘PBG’ refers to a Protein Binding Group.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

In this specification the term “alkylene” includes both straight and branched chain divalent alkyl groups. For example, “C₁₋₄alkylene” includes methylene (—CH₂—), ethylene (—CH₂CH₂—), propylene and butylene.

In this specification the term “alkoxy” includes both straight and branched chain alkyl groups singularly bonded to oxygen. For example, “C₁₋₄alkoxy” includes methoxy, ethoxy, isopropoxy and t-butoxy.

The term “(m-nC)” or “(m-nC) group” used alone or as a prefix, refers to any group having m to n carbon atoms.

“Cycloalkyl” means a hydrocarbon monocyclic or bicyclic ring containing carbon atoms. Examples of monocyclic cycloalkyl groups include cyclopropyl, cyclobutyl, cyclopentyl, cyclohexyl, cycloheptyl. Bicyclic rings may be fused or spiro attached; examples of bicyclic cycloalkyl groups include bicyclo[2.2.2]octane, bicyclo[2.1.1]hexane, bicyclo[1.1.1]pentane, spiro[2.4]heptane, bicyclo[4.1.0]heptane and bicyclo[2.2.1]heptane.

The term “halo” refers to fluoro, chloro, bromo and iodo.

The term “haloalkyl” is used herein to refer to an alkyl group respectively in which one or more hydrogen atoms have been replaced by halogen (e.g. fluorine) atoms. Examples of haloalkyl groups include fluoroalkyl groups such as —CHF₂, —CH₂CF₃, or perfluoroalkyl/alkoxy groups such as —CF₃, or —CF₂CF₃.

The term “heterocyclyl”, “heterocyclic” or “heterocycle” means a non-aromatic saturated or partially saturated monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, dihydroisoxazolyl (such as 4,5-dihydroisoxazolyl), dihydropyridinyl (such as 1,2-dihydropyridinyl or 1,6-dihydropyridinyl), tetrahydro-oxazolyl, tetrahydro-oxadiazolyl, tetrahydro-dioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O) or thioxo (=S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. Suitably, the term “heterocyclyl”, “heterocyclic” or “heterocycle” will refer to 4, 5, 6 or 7 membered monocyclic rings as defined above.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically, the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five. Suitably, the term “heteroaryl” or “heteroaromatic” will refer to 5 or 6 membered monocyclic heteroaryl rings as defined above.

Non-limiting examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl, 3,4-dihydro-2H-pyrido[3,2-b][1,4]oxazinyl, 4,5,6,7-tetrahydrobenzo[d]isoxazolyl, 4,5,6,7-tetrahydro-[1,2,3]triazolo[1,5-a]pyridinyl, 5,6-dihydro-8H-[1,2,4]triazolo[3,4-c][1,4]oxazinyl, 5,6-dihydro-4H-pyrrolo[1,2-c][1,2,3]triazolyl, 6,7-dihydro-5H-pyrrolo[2,1-c][1,2,4]triazolyl, 5,6,7,8-tetrahydro-[1,2,4]triazolo[4,3-a]pyridinyl, 6,7-dihydro-4H-[1,2,3]triazolo[5,1-c][1,4]-oxazinyl and 1,4,5,6-tetrahydrocyclopenta[d][1,2,3]triazol-5-yl.

Non-limiting examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Non-limiting examples of six membered heteroaryl groups include but are not limited to pyridyl, pyrazinyl, pyridazinyl, pyrimidinyl and triazinyl.

Particular non-limiting examples of bicyclic heteroaryl groups containing a six membered ring fused to a five membered ring include but are not limited to benzofuranyl, benzothiophenyl, benzimidazolyl, benzoxazolyl, benzisoxazolyl, benzothiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl, pyrrolopyridine, and pyrazolopyridinyl groups.

Particular non-limiting examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

Particular non-limiting examples of bicyclic heteroaryl groups containing a five membered ring fused to a five membered ring include but are not limited to 6,7-dihydro-5H-pyrrolo[2,1-c][1,2,4]triazolyl and 1,4,5,6-tetrahydrocyclopenta[d][1,2,3]triazol-5-yl.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In this particular embodiment, an aryl is phenyl or naphthyl, especially phenyl.

The term “carboxylic acid mimetic group” refers to surrogate structures or isosteres of the carboxylic acid group, which typically maintain the features of the carboxylic acid group needed for biological activity, but modify the physicochemical properties of the resultant compound, such as acidity or lipophilicity. Such carboxylic acid mimetic groups are known to those skilled in the art of medicinal chemistry. Examples of carboxylic acid mimetic groups include, but are not limited to, tetrazole, 3-trifluoromethyl-1,2,4-triazole, hydroxamic acids, hydroxamic esters, phosphonic acids, phosphinic acids, sulfonic acids, sulfinic acids, sulphonamides, sulfonyl ureas, acyl ureas, thiazolidine dione, oxazolidine dione, oxadiazol-5(4H)-one, thiadiazol-5(4H)-one, oxathiadiazole-2-oxide, oxadiazol-5(4H)-thione, isoxazole, tetramic acid, cyclopentane-1,3-diones and cyclopentane-1,2-diones.

The term “optionally substituted” refers to either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups.

The phrase “compound of the invention” means those compounds which are disclosed herein, both generically and specifically.

### Compounds of the Invention

In a first aspect, the present invention provides a compound of Formula I, or a pharmaceutically acceptable salt thereof:

KBG-L-PBG (I)
wherein L is a bivalent linker group covalently linking the KBG to the PBG; PBG is a Protein Binding Group comprising a moiety having a binding affinity to a target protein of interest; and
KBG is a KEAP1 Binding Group having the structure shown below:

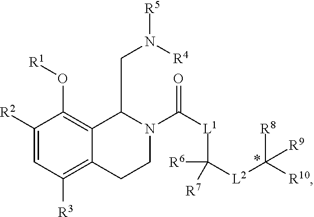

wherein:
R¹ is selected from C₁₋₄alkylene-R¹¹, heterocyclyl and 8-10 membered bicyclic heteroaryl, wherein said heterocyclyl is optionally substituted with one or more substituents independently selected from C₁₋₄alkyl, —C(O)—R¹², SO₂—R¹³, C₁₋₃alkylene-OR¹⁴ and heteroaryl which is optionally substituted with one or more substituents independently selected from C₁₋₄alkyl, C₃₋₇cycloalkyl, halo, OH, C₁₋₃alkoxy and cyano; and wherein said 8-10 membered bicyclic heteroaryl is optionally substituted with one or more substituents independently selected from C₁₋₄alkyl, C₃₋₇cycloalkyl, halo, OH and C₁₋₃alkoxy;
R² is selected from hydrogen, fluoro, chloro and C₁₋₃alkyl;
R³ is selected from hydrogen, fluoro, chloro, bromo, C₁₋₃alkoxy, C₁₋₃alkyl, C₁₋₃haloalkyl and cyano;
R⁴ is hydrogen or C₁₋₄alkyl;
R⁵ is —C(O)—C₁₋₄alkyl, —C(O)-heteroaryl or —C(O)-aryl, wherein said heteroaryl and aryl are optionally substituted with one or more substituents selected from C₁₋₄alkyl, halo, hydroxy, C₁₋₃alkoxy, CO₂R¹⁵ and cyano; or
R⁴ and R⁵, taken together with the nitrogen atom to which they are attached, form a 4-, 5- or 6-membered heterocyclyl ring, wherein said heterocyclyl ring:
   comprises one or more —C(O)— moieties attached to the nitrogen atom;
   optionally is fused to an aryl or heteroaryl ring;
   optionally is spiro-attached to a C₃₋₇cycloalkyl group; and
   optionally is substituted with one or more substituents independently selected from C₁₋₄alkyl, halo, OH, C₁₋₃alkoxy, C₁₋₃haloalkyl, cyano, NR¹⁶R¹⁷, C(O)R¹, S(O)R¹⁹ and SO₂R²⁰;
L¹ and L² are independently selected from a bond and —CR²¹R²²—;
R⁶ and R⁷ are independently selected from hydrogen, C₁₋₄alkyl, and C₃₋₇cycloalkyl; or
R⁶ and R⁷, taken together with the carbon atom to which they are attached, form a 3-, 4-, 5-, or 6-membered cycloalkyl ring;
R⁸ is selected from C(═O)NR^{3a}R^{3b}, —C(═O)R^{8c}, CO₂R²³, C(O)NHSO₂C₁₋₃alkyl, tetrazolyl, 3-trifluoromethyl-1,2,4-triazol-5-yl, and a carboxylic acid mimetic group selected from hydroxamic acids, hydroxamic esters, phosphonic acids, phosphinic acids, sulfonic acids, sulfinic acids, sulphonamides, sulfonyl ureas, acyl ureas, thiazolidine dione, oxazolidine dione, oxadiazol-5(4H)-one, thiadiazol-5(4H)-one, oxathiadiazole-2-oxide, oxadiazol-5(4H)-thione, isoxazole, tetramic acid, cyclopentane-1,3-diones and cyclopentane-1,2-diones;
R^{8a} is hydrogen or C₁₋₆alkyl;
R^{8b} is hydrogen, C₁₋₆alkyl or C₃₋₇cycloalkyl, wherein the C₁₋₆alkyl or C₃₋₇cycloalkyl groups are optionally substituted with one or more substituents independently selected from C₁₋₄alkyl, C₃₋₇cycloalkyl, halo, OH, C₁₋₃alkoxy, C₁₋₃haloalkyl, cyano, NR¹⁶R¹⁷, C(O)R¹⁸, S(O)R¹⁹ and SO₂R²⁰; or
R^{8a} and R^{8b}, taken together with the nitrogen atom to which they are attached, form a 3-, 4-, 5-, 6-, or 7-membered heterocyclyl ring, wherein the heterocyclyl ring optionally contains one or more additional heteroatoms selected from oxygen, nitrogen and sulfur, and is optionally substituted with one or more substituents independently selected from C₁₋₄alkyl, C₃₋₇cycloalkyl, halo, OH, C₁₋₃alkoxy, C₁₋₃haloalkyl, cyano, NR¹⁶R¹⁷, C(O)R¹⁸, S(O)R¹⁹ and SO₂R²⁰;
R^{8c} is C₁₋₃alkyl or C₁₋₃haloalkyl;
R⁹ is selected from hydrogen, C₁₋₄alkyl, hydroxy, C₁₋₃alkoxy and halo;
R¹⁰ is selected from hydrogen and C₁₋₄alkyl; or
R⁹ and R¹⁰, taken together with the carbon atom to which they are attached, form a 3-, 4-, 5-, or 6-membered cycloalkyl ring; or
L² is a bond and R⁷ and R¹⁰, taken together with the atoms to which they are attached, form a 4-, 5-, 6- or 7-membered cycloalkyl or heterocyclyl ring, wherein:
   said heterocyclyl ring contains 1 or 2 heteroatoms independently selected from nitrogen, oxygen and sulfur;
   said cycloalkyl ring optionally comprises 1 or 2 carbon-carbon double bonds and is optionally bridged by a C₁₋₃alkylene group connecting two carbon atoms of the ring, or R⁹ is optionally a C₁₋₃alkylene group connecting C* to a carbon atom of the ring; and
   said cycloalkyl and heterocyclyl rings are optionally substituted with one or more substituents independently selected from C₁₋₄alkyl, halo, OH, C₁₋₃alkoxy, C₁₋₃haloalkyl and deuterium;
R¹¹ is selected from —C(O)—R²⁴, —SO₂—R²⁵, —NR²⁶C(O)—R²⁷, —NR²⁸SO₂—R²⁹, heterocyclyl, aryl and heteroaryl, wherein said heterocyclyl, aryl and heteroaryl groups are optionally substituted with one or more substituents independently selected from C₁₋₄alkyl, C₁₋₃haloalkyl, C₃₋₇cycloalkyl, C₁₋₄alkylene-R³⁰, halo, OH, C₁₋₃alkoxy, heterocyclyl and cyano; and said heterocyclyl group is optionally substituted with one or more substituents independently selected from C₁₋₄alkyl, C₁₋₃haloalkyl, C₃₋₇cycloalkyl, C₁₋₄alkylene-R³⁰, halo, OH, C₁₋₃alkoxy, oxo and cyano;
R¹² is selected from C₁₋₄alkyl, C₃₋₇cycloalkyl, OR³¹, NR³²R³³, aryl and heteroaryl, wherein said aryl and heteroaryl are optionally substituted with one or more substituents independently selected from C₁₋₄alkyl, halo, OH, C₁₋₃alkoxy and cyano;
R¹³ is selected from C₁₋₄alkyl, C₃₋₇cycloalkyl, heteroaryl, heterocyclyl and NR³⁴R³⁵, wherein said heteroaryl and heterocyclyl are optionally substituted with one or more substituents independently selected from C₁₋₄alkyl, halo, OH, C₁₋₃alkoxy and cyano;
R¹⁷ is selected from hydrogen, C₁₋₄alkyl, C(O)C₁₋₃alkyl and C(O)NR³⁶R³⁷;
R¹⁸, R¹⁹ and R²⁰ are independently selected from C₁₋₄alkyl, OH, C₁₋₃alkoxy and NR³⁸R³⁹;
R²³ is selected from hydrogen and C₁₋₄alkyl;
R²⁴ is selected from C₁₋₄alkyl, NR⁴⁰R⁴¹ and OR⁴²;
R²⁵ is selected from C₁₋₄alkyl and NR⁴³R⁴⁴;
R²⁷ is selected from C₁₋₄alkyl, C₃₋₇cycloalkyl, C₁₋₃haloalkyl, heterocyclyl, aryl and heteroaryl, wherein said aryl and heteroaryl are optionally substituted with one or more substituents independently selected from C₁₋₄alkyl, C₁₋₃haloalkyl, C₃₋₇ cycloalkyl, C₁₋₄alkylene-R⁴⁵, halo, OH, C₁₋₃alkoxy and cyano;
R²⁹ is selected from C₁₋₄alkyl, C₃₋₇cycloalkyl, C₁₋₃haloalkyl, aryl and heteroaryl, wherein said aryl and heteroaryl are optionally substituted with one or more substituents independently selected from C₁₋₄alkyl, C₁₋₃haloalkyl, C₃₋₇cycloalkyl, C₁₋₄alkylene-R⁴⁶ halo, OH, C₁₋₃alkoxy and cyano;
R³⁰ is selected from hydroxy, C₁₋₃alkoxy, C₃₋₇cycloalkyl, cyano and NR⁴⁷R⁴⁸;
R⁴⁰ is selected from hydrogen and C₁₋₄alkyl;
R⁴¹ is selected from hydrogen, C₁₋₄alkyl, C₃₋₇cycloalkyl, C₁₋₃alkoxy, aryl and heteroaryl; or
R⁴⁰ and R⁴¹, taken together with the nitrogen atom to which they are attached, form a 4-, 5-, or 6-membered heteroaryl or heterocyclyl ring, wherein said heteroaryl and heterocyclyl rings are optionally substituted with one or more substituents independently selected from C₁₋₄alkyl, halo, OH, C₁₋₃alkoxy, C₃₋₇cycloalkyl and cyano;
R⁴⁵ and R⁴⁶ are independently selected from hydroxy, C₁₋₃alkoxy and C₃₋₇cycloalkyl; and
R¹⁴, R¹⁵, R¹⁶, R²¹, R²², R²⁶, R²⁸, R³¹, R³², R³³, R³⁴, R³⁵, R³⁶, R³⁷, R³⁸, R³⁹, R⁴², R⁴³, R⁴⁴, R⁴⁷ and R⁴⁸ are independently selected from hydrogen, C₁₋₄alkyl and C₃₋₇cycloalkyl.

Particular compounds of the invention include, for example, compounds of the formula I, or pharmaceutically acceptable salts thereof, wherein, unless otherwise stated, each of R¹, R², R³, R⁴, R⁵, L¹, L², R⁶, R⁷, R⁸, R^{8a}, R^{8b}, R^{8c}, R⁹, R¹⁰, R¹¹, R¹², R¹³, R¹⁷, R¹⁸, R¹⁹, R²⁰, R²⁴, R²⁵, R²⁷, R²⁹, R³⁰, R⁴⁰, R⁴¹, L, or PBG has any of the meanings defined hereinbefore, or in any of paragraphs (1) to (126) hereinafter. For the avoidance of doubt, the invention encompasses any and all combinations of two or more definitions as described in paragraphs (1) to (126):
(1) R¹ is C₁₋₄alkylene-R¹¹;
(2) R¹ is CH₂—R¹¹;
(3) R¹ is CH₂CH₂—R¹¹;
(4) R¹ is CH(Me)-R¹¹;
(5) R¹ is heterocyclyl, optionally substituted with one or more substituents independently selected from C₁₋₄alkyl, —C(O)—R¹², SO₂—R¹³, heteroaryl and C₁₋₃alkylene-OR¹⁴, wherein said heteroaryl is optionally substituted with one or more substituents independently selected from C₁₋₄alkyl, C₃₋₇cycloalkyl, halo, OH, C₁₋₃alkoxy and cyano;
(6) R¹ is heterocyclyl, optionally substituted with one or more substituents independently selected from C₁₋₄alkyl, —C(O)—R¹², SO₂—R¹³, heteroaryl and C₁₋₃alkylene-OR¹⁴, wherein said heteroaryl is optionally substituted with one or more substituents independently selected from C₁₋₄alkyl and C₃₋₇cycloalkyl;
(7) R¹ is piperidinyl or pyrrolidinyl, each optionally substituted with one or more substituents independently selected from —C(O)—R¹², SO₂—R¹³, heteroaryl and C₁₋₃alkylene-OR¹⁴, wherein said heteroaryl is optionally substituted with one or more substituents independently selected from C₁₋₄alkyl, C₃₋₇cycloalkyl, halo, OH, C₁₋₃alkoxy and cyano;
(8) R¹ is pyrrolidinyl, optionally substituted with one or more substituents independently selected from —C(O)—R¹², SO₂—R¹³, heteroaryl and C₁₋₃alkylene-OR¹⁴, wherein said heteroaryl is optionally substituted with C₁₋₄alkyl or C₃₋₇cycloalkyl;
(9) R¹ is selected from one of the following groups:

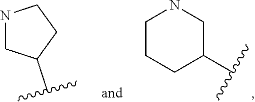

wherein [symbol] represents the point of attachment of the group to the oxygen atom of the rest of the compound and wherein each group is optionally substituted with one or more substituents independently selected from —C(O)—R¹², SO₂—R¹³, heteroaryl and C₁₋₃alkylene-OR¹⁴, wherein said heteroaryl is optionally substituted with C₁₋₄alkyl or C₃₋₇cycloalkyl;
(10) R¹ is an 8-10 membered bicyclic heteroaryl optionally substituted with one or more substituents independently selected from C₁₋₄alkyl, C₃₋₇cycloalkyl, halo, OH and C₁₋₃alkoxy;
(11) R¹ is an 8 membered bicyclic heteroaryl optionally substituted with one or more substituents independently selected from C₁₋₄alkyl, OH and C₁₋₃alkoxy;
(12) R¹ is selected from one of the following groups:

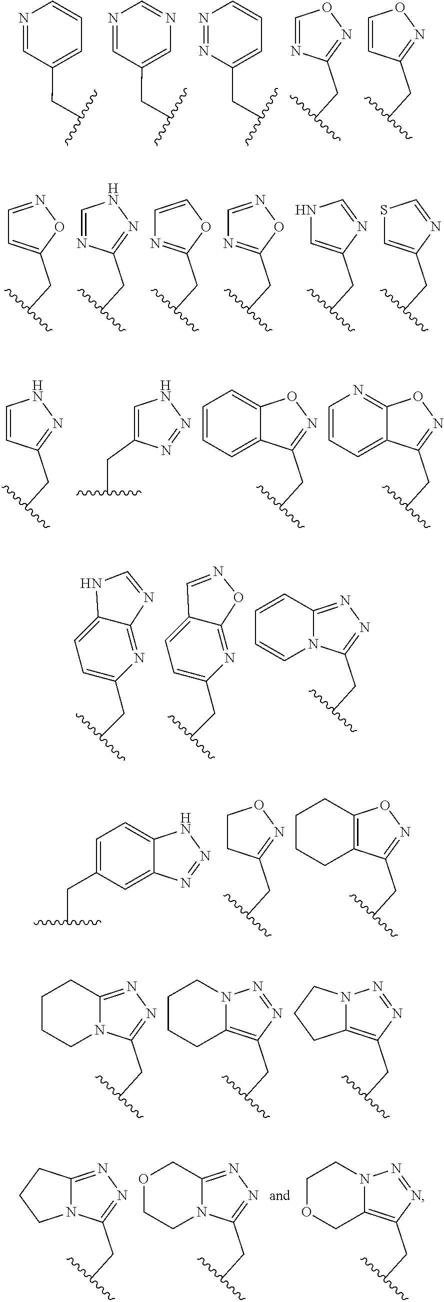

wherein [symbol] represents the point of attachment of the group to the oxygen atom of the rest of the compound and wherein each cyclic group is optionally substituted with one or more substituents independently selected from C₁₋₄alkyl, C₁₋₃haloalkyl, C₃₋₇cycloalkyl, C₁₋₄alkylene-R³⁰, halo, OH, C₁₋₃alkoxy, heterocycloalkyl and cyano;
(13) R² is selected from hydrogen, fluoro and chloro;
(14) R² is hydrogen or fluoro;
(15) R³ is selected from hydrogen, chloro, bromo, C₁₋₃alkoxy, C₁₋₃alkyl, C₁₋₃haloalkyl and cyano;
(17) R³ is selected from hydrogen, chloro, bromo, methoxy, methyl, trifluoromethyl and cyano;
(18) R³ is selected from hydrogen and chloro;
(19) R³ is chloro;
(20) R⁴ is hydrogen;
(21) R⁵ is —C(O)—C₁₋₄alkyl or —C(O)-aryl, wherein said aryl group is optionally substituted with one or more substituents selected from C₁₋₄alkyl, halo, hydroxy, C₁₋₃alkoxy, CO₂R¹⁵ and cyano;
(22) R⁴ is hydrogen and R⁵ is —C(O)—C₁₋₄alkyl or —C(O)-aryl, wherein said aryl group is optionally substituted with one or more substituents selected from C₁₋₄alkyl, halo, hydroxy, C₁₋₃alkoxy, CO₂R¹⁵ and cyano;
(23) R⁴ is hydrogen and R⁵ is —C(O)—C₁₋₄alkyl or —C(O)-aryl, wherein said aryl group is optionally substituted with one or more substituents selected from halo, hydroxy and CO₂R¹⁵;
(24) R⁴ and R⁵, taken together with the nitrogen atom to which they are attached, form a 4-, 5-, or 6-membered heterocyclyl ring, wherein said heterocyclyl ring comprises one or more —C(O)— moieties attached to the nitrogen atom and is optionally fused to an aryl ring, or optionally spiro-attached to a C₃₋₇cycloalkyl group; and said heterocyclyl ring is optionally substituted with one or more substituents independently selected from C₁₋₄alkyl, halo, OH, C₁₋₃alkoxy, C₁₋₃haloalkyl, cyano, NR¹⁶R¹⁷, C(O)R¹⁸, S(O)R¹⁹ and SO₂R²⁰;
(25) R⁴ and R⁵, taken together with the nitrogen atom to which they are attached, form a 5-membered heterocyclyl ring, wherein said heterocyclyl ring comprises a —C(O)— moiety attached to the nitrogen atom and is optionally fused to an aryl ring, or optionally spiro-attached to a C₃₋₇cycloalkyl group; and said heterocyclyl ring is optionally substituted with one or more substituents independently selected from C₁₋₄alkyl, halo, OH, C₁₋₃alkoxy and cyano;
(26) R⁴ and R⁵, taken together with the nitrogen atom to which they are attached, form a 5- or 6-membered heterocyclyl ring, wherein said heterocyclyl ring comprises a —C(O)— moiety attached to the nitrogen atom and is optionally fused to an aryl ring, or optionally spiro-attached to a C₃₋₇cycloalkyl group; and said heterocyclyl ring is optionally substituted with one or more substituents independently selected from C₁₋₄alkyl, halo and OH;
(27) R⁴ and R⁵, taken together with the nitrogen atom to which they are attached, form a 5-membered heterocyclyl ring, wherein said heterocyclyl ring comprises a —C(O)— moiety attached to the nitrogen atom and is optionally fused to a phenyl ring, or optionally spiro-attached to a cyclopropyl group; and said heterocyclyl ring is optionally substituted with one or more substituents independently selected from methyl, fluoro and OH;
(28) R⁴ and R⁵, taken together with the nitrogen atom to which they are attached, form a heterocyclic moiety selected from one of the following:

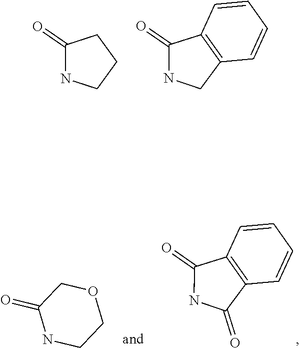

wherein the saturated ring of the heterocyclic moiety is optionally spiro-attached to a C₃₋₇cycloalkyl group, and wherein said heterocyclic moiety is optionally substituted with one or more substituents independently selected from C₁₋₄alkyl, halo and OH;
(29) R⁴ and R⁵, taken together with the nitrogen atom to which they are attached, form a heterocyclic moiety selected from one of the following:

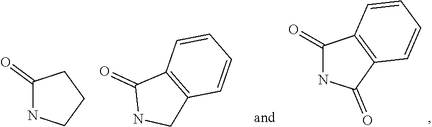

wherein the saturated ring of the heterocyclic moiety is optionally spiro-attached to a cyclopropyl group, and wherein said heterocyclic moiety is optionally substituted with one or more substituents independently selected from methyl, fluoro and OH;
(30) R⁴ and R⁵, taken together with the nitrogen atom to which they are attached, form the following heterocyclic moiety:

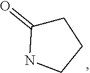

wherein the heterocyclic moiety is optionally spiro-attached to a cyclopropyl group, or is optionally substituted with one or more substituents independently selected from methyl and fluoro;
(31) R⁴ and R⁵, taken together with the nitrogen atom to which they are attached, form the following heterocyclic moiety:

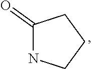

wherein the heterocyclic moiety is optionally spiro-attached to a cyclopropyl group, and is optionally substituted with C₁₋₃alkyl, wherein said cyclopropyl and/or C₁₋₃alkyl group is attached to the heterocyclic ring at a position either alpha or beta to the carbonyl group;
(32) R⁴ and R⁵, taken together with the nitrogen atom to which they are attached, form the following moiety:

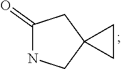

(33) L¹ and L² are independently selected from a bond and —CH₂—;
(34) L¹ is a bond and L² is —CH₂—;
(35) L¹ is CH₂— and L² is a bond;
(36) L¹ and L² are both bonds;
(37) R⁶ and R⁷ are independently selected from hydrogen and C₁₋₄alkyl;
(38) R⁶ and R⁷ are independently selected from hydrogen and methyl;
(39) R⁶ and R⁷ are both hydrogen;
(40) R⁶ and R⁷, taken together with the carbon atom to which they are attached, form a 3-, 4-, 5-, or 6-membered cycloalkyl ring;
(41) R³ is selected from C(═O)NR^{8a}R^{8b}, —C(═O)R^{8c}, CO₂R²³, C(O)NHSO₂C₁₋₃alkyl, tetrazolyl and 3-trifluoromethyl-1,2,4-triazol-5-yl;
(42) R³ is selected from C(═O)NR^{8a}R^{8b}, —C(═O)R^{8c}, CO₂H and tetrazolyl;
(43) R⁸ is selected from C(═O)NR^{8a}R^{8b} and CO₂H;
(44) R⁸ is CO₂H;
(45) R⁸ is C(═O)NR^{8a}R^{8b};
(46) R⁸ is —C(═O)R^{8c};
(47) R^{8a} is hydrogen or methyl;
(48) R^{8a} is hydrogen;
(49) R^{8b} is hydrogen, C₁₋₆alkyl or C₃₋₇cycloalkyl, wherein the C₁₋₆alkyl group is optionally substituted with one or more substituents independently selected from halo, OH, C₁₋₃alkoxy, C₃₋₇cycloalkyl, cyano, NR¹⁶R¹⁷, C(O)R¹⁸, S(O)R¹⁹ and SO₂R²⁰;
(50) R^{8b} is hydrogen, C₁₋₄alkyl or C₃₋₅cycloalkyl, wherein the C₁₋₄alkyl group is optionally substituted with one or more substituents independently selected from halo, OH, C₁₋₃alkoxy C₃₋₇cycloalkyl and cyano;
(51) R^{8b} is C₁₋₄alkyl or C₃₋₅cycloalkyl, wherein the C₁₋₄alkyl group is optionally substituted with one or more substituents independently selected from fluoro, OH, C₁₋₃alkoxy C₃₋₇cycloalkyl and cyano;
(52) R^{8b} is hydrogen, methyl, ethyl, n-propyl, cyclopropyl, cyclobutyl or cyclopropylmethyl wherein the methyl, ethyl or n-propyl groups are optionally substituted with one or more substituents independently selected from fluoro, OH, methoxy and cyano;
(53) R^{8b} is methyl, ethyl, n-propyl, cyclopropyl, cyclobutyl or cyclopropylmethyl;
(54) R^{8b} is methyl;
(55) R^{8a} is hydrogen and R^{8b} is hydrogen, C₁₋₆alkyl or C₃₋₇cycloalkyl, wherein the C₁₋₆alkyl group is optionally substituted with one or more substituents independently selected from halo, OH, C₁₋₃alkoxy, C₃₋₇cycloalkyl, cyano, NR¹⁶R¹⁷, C(O)R¹⁸, S(O)R¹⁹ and SO₂R²⁰;
(56) R^{8a} is hydrogen and R^{8b} is C₁₋₆alkyl or C₃₋₇cycloalkyl, wherein the C₁₋₆alkyl group is optionally substituted with one or more substituents independently selected from fluoro, OH, C₁₋₃alkoxy, C₃₋₇cycloalkyl, cyano, NR¹⁶R¹⁷, C(O)R¹⁸, S(O)R¹⁹ and SO₂R²⁰;
(57) R^{8a} is hydrogen and R^{8b} is methyl, ethyl, n-propyl, cyclopropyl, cyclobutyl or cyclopropylmethyl;
(58) R^{8a} is hydrogen and R^{8b} is methyl;
(59) R^{8a} and R^{8b}, taken together with the nitrogen atom to which they are attached, form a 3-, 4-, or 5-membered heterocyclyl ring, wherein the heterocyclyl ring optionally contains one or more additional heteroatoms selected from oxygen, nitrogen and sulfur, and is optionally substituted with one or more substituents independently selected from C₁₋₄alkyl, halo, OH, C₁₋₃alkoxy, C₁₋₃haloalkyl, C₃₋₇cycloalkyl, cyano, NR¹⁶R¹⁷, C(O)R¹⁸, S(O)R¹⁹ and SO₂R²⁰;
(60) R^{8a} and R^{3b}, taken together with the nitrogen atom to which they are attached, form a 4-, or 5-membered heterocyclyl ring, wherein the heterocyclyl ring is optionally substituted with one or more substituents independently selected from C₁₋₄alkyl, halo, OH, C₁₋₃alkoxy, C₁₋₃haloalkyl, C₃₋₇cycloalkyl, cyano, NR¹⁶R¹⁷, C(O)R¹⁸, S(O)R¹⁹ and SO₂R²⁰;
(61) R^{8a} and R^{3b}, taken together with the nitrogen atom to which they are attached, form an azetidinyl ring optionally substituted with one or more substituents independently selected from methyl, fluoro, OH, methoxy and C₁₋₃fluoroalkyl;
(62) R⁸⁰ is C₁₋₃alkyl or C₁₋₃fluoroalkyl;
(63) R⁸⁰ is C₁₋₃fluoroalkyl;
(64) R^{8c} is fluoromethyl, difluoromethyl or trifluoromethyl;
(65) R⁹ is selected from hydrogen, C₁₋₄alkyl, C₁₋₃alkoxy and halo;
(66) R⁹ is selected from hydrogen, methyl, methoxy and fluoro;
(67) R⁹ is selected from C₁₋₄alkyl, C₁₋₃alkoxy and halo;
(68) R⁹ is selected from methyl, methoxy and fluoro;
(69) R⁹ is hydrogen or C₁₋₄alkyl;
(70) R⁹ is hydrogen or methyl;
(71) R⁹ is methyl;
(72) R¹⁰ is selected from hydrogen and methyl;
(73) R⁹ and R¹⁰, taken together with the carbon atom to which they are attached, form a 3-, 4-, 5-, or 6-membered cycloalkyl ring;
(74) L² is a bond and R⁷ and R¹⁰, taken together with the atoms to which they are attached, form a 4-, 5- or 6-membered cycloalkyl or heterocyclyl ring, wherein: said heterocyclyl ring contains 1 or 2 heteroatoms independently selected from nitrogen, oxygen and sulfur;
   said cycloalkyl ring optionally comprises 1 or 2 carbon-carbon double bonds and is optionally bridged by a C₁₋₃alkylene group connecting two carbon atoms of the ring, or R⁹ is optionally a C₁₋₃alkylene group connecting C* to a carbon atom of the ring; and
   said cycloalkyl and heterocyclyl rings are optionally substituted with one or more substituents independently selected from C₁₋₄alkyl, halo, OH, C₁₋₃alkoxy and deuterium;
(75) L¹ and L² are both bonds and R⁷ and R¹⁰, taken together with the atoms to which they are attached, form a 4-, 5- or 6-membered cycloalkyl or heterocyclyl ring, wherein: said heterocyclyl ring contains 1 or 2 heteroatoms independently selected from nitrogen, oxygen and sulfur;
   said cycloalkyl ring optionally comprises a carbon-carbon double bond and is optionally bridged by a C₁₋₃alkylene group connecting two carbon atoms of the ring, or R⁹ is optionally a C₁₋₃alkylene group connecting C* to a carbon atom of the ring; and
   said cycloalkyl and heterocyclyl rings are optionally substituted with one or more substituents independently selected from C₁₋₄alkyl, halo, OH, C₁₋₃alkoxy and deuterium;
(76) L² is a bond and R⁷ and R¹⁰, taken together with the atoms to which they are attached, form a 4-, 5- or 6-membered cycloalkyl ring, wherein said cycloalkyl ring is optionally bridged by a C₁₋₃alkylene group connecting two carbon atoms of the ring, or R⁹ is optionally a C₁₋₃alkylene group connecting C* to a carbon atom of the ring, and said cycloalkyl ring is optionally substituted with one or more substituents independently selected from C₁₋₄alkyl, halo, OH, C₁₋₃alkoxy and deuterium;
(77) L² is a bond and R⁷ and R¹⁰, taken together with the atoms to which they are attached, form a cyclohexyl ring, wherein said cyclohexyl ring optionally comprises a carbon-carbon double bond and is optionally bridged by a C₁₋₃alkylene group connecting two carbon atoms of the ring, or R⁹ is optionally a C₁₋₃alkylene group connecting C* to a carbon atom of the ring, and said cyclohexyl ring is optionally substituted with one or more substituents independently selected from C₁₋₄alkyl, halo, OH and deuterium;
(78) R¹¹ is selected from —C(O)—R²⁴, —NR²¹C(O)—R²⁷, heterocyclyl, and heteroaryl wherein said heterocyclyl and heteroaryl groups are optionally substituted with one or more substituents independently selected from C₁₋₄alkyl, C₁₋₃haloalkyl, C₃₋₇cycloalkyl, C₁₋₄alkylene-R³⁰, halo, OH, C₁₋₃alkoxy, heterocyclyl and cyano;
(79) R¹¹ is —C(O)—R²⁴ and R²⁴ is selected from NR⁴⁰R⁴¹ and OR⁴²;
(80) R¹¹ is —NR²⁶C(O)—R²⁷ and R²⁷ is selected from C₁₋₄alkyl, C₃₋₇cycloalkyl, heterocyclyl, aryl and heteroaryl, wherein said aryl and heteroaryl are optionally substituted with one or more substituents independently selected from C₁₋₄alkyl and C₃₋₇cycloalkyl;
(81) R¹¹ is heteroaryl optionally substituted with one or more substituents independently selected from C₁₋₄alkyl, C₁₋₃haloalkyl, C₃₋₇cycloalkyl, C₁₋₄alkylene-R³¹, halo, OH, C₁₋₃alkoxy, heterocyclyl and cyano;
(82) R¹¹ is heteroaryl optionally substituted with one or more substituents independently selected from C₁₋₄alkyl, C₁₋₃haloalkyl, C₃₋₇cycloalkyl, C₁₋₄alkylene-R³⁰, C₁₋₃alkoxy, and cyano;
(83) R¹¹ is heteroaryl optionally substituted with one or more substituents independently selected from methyl, ethyl, isopropyl, difluoromethyl, trifluoromethyl, chloro, fluoro, cyclopropyl, methoxy, cyano, oxetanyl, CH₂—R³⁰ and CH₂CH₂—R³⁰;
(84) R¹¹ is pyridyl, pyrimidinyl, pyridazinyl, oxazolyl, isoxazolyl, 1,2,3-triazolyl, 1,2,4-oxadiazolyl, benzotriazolyl, benzisoxazolyl, isoxazolopyridinyl, imidazopyridinyl or triazolopyridinyl, each optionally substituted with one or more substituents independently selected from C₁₋₄alkyl, C₁₋₃haloalkyl, C₃₋₇cycloalkyl, C₁₋₄alkylene-R³⁰, halo, OH, C₁₋₃alkoxy, heterocyclyl and cyano;
(85) R¹¹ is selected from:

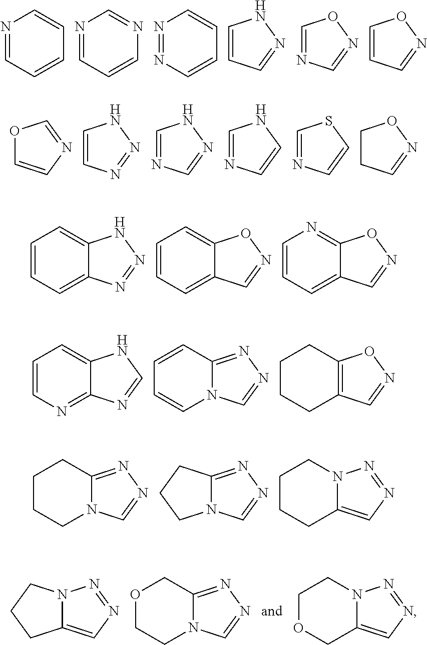

optionally substituted with one or more substituents independently selected from C₁₋₄alkyl, C₁₋₃haloalkyl, C₃₋₇cycloalkyl, C₁₋₄alkylene-R³⁰, halo, OH, C₁₋₃alkoxy, heterocycloalkyl and cyano;
(86) R¹¹ is oxazolyl, isoxazolyl or 1,2,3-triazolyl, each optionally substituted with one or more substituents independently selected from C₁₋₄alkyl, C₁₋₃haloalkyl, C₃₋₇cycloalkyl, C₁₋₄alkylene-R³⁰, halo, OH, C₁₋₃alkoxy, heterocycloalkyl and cyano;
(87) R¹¹ is 1,2,3-triazolyl optionally substituted with one or more substituents independently selected from C₁₋₄alkyl, C₁₋₃haloalkyl, C₃₋₇cycloalkyl, C₁₋₄alkylene-R³⁰, halo, OH, C₁₋₃alkoxy, heterocycloalkyl and cyano;
(88) R¹¹ is heterocyclyl optionally substituted with one or more substituents independently selected from C₁₋₄alkyl, C₁₋₃haloalkyl, C₃₋₇cycloalkyl, C₁₋₄alkylene-R³⁰, halo, OH, C₁₋₃alkoxy, oxo and cyano;
(89) R¹¹ is heterocyclyl optionally substituted with one or more substituents independently selected from C₁₋₄alkyl, C₁₋₃haloalkyl, halo, OH and oxo;
(90) R²⁹ is selected from C₁₋₄alkyl, C₃₋₇cycloalkyl, C₁₋₃haloalkyl and heteroaryl, wherein said heteroaryl is optionally substituted with one or more substituents independently selected from C₁₋₄alkyl and C₁₋₃haloalkyl;
(91) R²⁹ is methyl, ethyl or cyclopropyl;
(92) R³⁰ is selected from hydroxy, C₁₋₃alkoxy, C₃₋₇cycloalkyl and cyano;
(93) R³⁰ is selected from hydroxy, methoxy, cyclopropyl and cyano;
(94) R⁴⁰ is selected from hydrogen and methyl;
(95) R⁴¹ is selected from hydrogen, methyl, cyclopropyl, methoxy and phenyl;
(96) R⁴⁰ and R⁴¹, taken together with the nitrogen atom to which they are attached, form 5-membered heteroaryl or heterocyclyl ring, wherein said heteroaryl and heterocyclyl rings are optionally substituted with one or more substituents independently selected from C₁₋₄alkyl, halo, OH, C₁₋₃alkoxy, C₃₋₇cycloalkyl and cyano;
(97) R⁴⁰ and R⁴¹, taken together with the nitrogen atom to which they are attached, form 5-membered heterocyclyl ring, wherein said heterocyclyl ring is optionally substituted with one or more substituents independently selected from C₁₋₄alkyl, halo and OH;
(98) R⁴⁰ and R⁴¹, taken together with the nitrogen atom to which they are attached, form 5-membered heterocyclyl ring, wherein said heterocyclyl ring is optionally substituted with one or more substituents independently selected from methyl, fluoro and OH;
(99) L is a covalent bond or a bivalent, saturated or unsaturated, straight or branched hydrocarbon chain, comprising 1-50 carbon atoms; wherein
   between 0 and 6 alkylene units of L are independently replaced by -Cy-, —O—, —N(R), —S—, —OC(O)—, —C(O)O—, —S(O)—, —S(O)₂—, —N(R)S(O)₂—, —S(O)₂N(R)—, —N(R)C(O)—, —C(O)N(R)—, —OC(O)N(R)—,

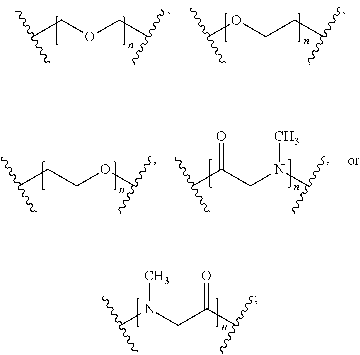

and
each -Cy- is independently an optionally substituted bivalent ring selected from:
   i) phenylenyl,
   ii) an 8-10 membered bicyclic arylenyl,
   iii) a 4-7 membered saturated or partially unsaturated carbocyclenyl,
   iv) a 4-7 membered saturated or partially unsaturated spiro carbocyclylenyl,
   v) an 8-10 membered bicyclic saturated or partially unsaturated carbocyclylenyl,
   vi) a 4-7 membered saturated or partially unsaturated heterocyclylenyl having 1-2 heteroatoms independently selected from nitrogen, oxygen, and sulfur,
   vii) a 4-7 membered saturated or partially unsaturated spiro heterocyclylenyl having 1-2 heteroatoms independently selected from nitrogen, oxygen or sulfur,
   viii) an 8-10 membered bicyclic saturated or partially unsaturated heterocyclylenyl having 1-2 heteroatoms independently selected from nitrogen, sulfur or oxygen,
   ix) a 5-6 membered heteroarylenyl having 1-4 heteroatoms independently selected from nitrogen, oxygen and sulfur, and
   x) an 8-10 membered bicyclic heteroarylenyl having 1-5 heteroatoms independently selected from nitrogen, oxygen or sulfur;
each n is independently 1-10; and
each R is independently hydrogen, or an optionally substituted group selected from C₁₋₆alkyl, phenyl, a 4-7 membered saturated or partially unsaturated heterocyclic having 1-2 heteroatoms independently selected from nitrogen, oxygen and sulfur, and a 5-6 membered heteroaryl ring having 1-4 heteroatoms independently selected from nitrogen, oxygen and sulfur;
(100) L is a bivalent, saturated or unsaturated, straight or branched hydrocarbon chain, comprising 1-20 carbon atoms; wherein
   between 0 and 6 alkylene units of L are independently replaced by -Cy-, —O—, —N(R), —S—, —OC(O)—, —C(O)O—, —S(O)—, —S(O)₂—, —N(R)S(O)₂—, —S(O)₂N(R)—, —N(R)C(O)—, —C(O)N(R)—, —OC(O)N(R)—,

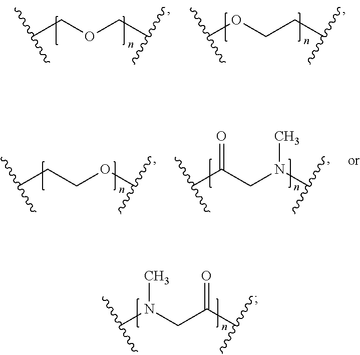

and
each -Cy- is independently an optionally substituted bivalent ring selected from:
   i) phenylenyl,
   ii) an 8-10 membered bicyclic arylenyl,
   iii) a 4-7 membered saturated or partially unsaturated carbocyclenyl,
   iv) a 4-7 membered saturated or partially unsaturated spiro carbocyclylenyl,
   v) an 8-10 membered bicyclic saturated or partially unsaturated carbocyclylenyl,
   vi) a 4-7 membered saturated or partially unsaturated heterocyclylenyl having 1-2 heteroatoms independently selected from nitrogen, oxygen, and sulfur,
   vii) a 4-7 membered saturated or partially unsaturated spiro heterocyclylenyl having 1-2 heteroatoms independently selected from nitrogen, oxygen or sulfur,
   viii) an 8-10 membered bicyclic saturated or partially unsaturated heterocyclylenyl having 1-2 heteroatoms independently selected from nitrogen, sulfur or oxygen,
   ix) a 5-6 membered heteroarylenyl having 1-4 heteroatoms independently selected from nitrogen, oxygen and sulfur, and
   x) an 8-10 membered bicyclic heteroarylenyl having 1-5 heteroatoms independently selected from nitrogen, oxygen or sulfur;
each n is independently 1-10; and
each R is independently hydrogen, or an optionally substituted group selected from C₁₋₆alkyl, phenyl, a 4-7 membered saturated or partially unsaturated heterocyclic having 1-2 heteroatoms independently selected from nitrogen, oxygen and sulfur, and a 5-6 membered heteroaryl ring having 1-4 heteroatoms independently selected from nitrogen, oxygen and sulfur;
(101) L is a bivalent, saturated or unsaturated, straight or branched hydrocarbon chain, comprising 1-20 carbon atoms; wherein between 0 and 6 alkylene units of L are independently replaced by —O—, —N(R), —S—, —OC(O)—, —C(O)O—, —S(O)—, —S(O)₂—, —N(R)S(O)₂—, —S(O)₂N(R)—, —N(R)C(O)—, —C(O)N(R)—, —OC(O)N(R)—, 4-7 membered saturated or partially unsaturated heterocyclylenyl having 1-2 heteroatoms independently selected from nitrogen, oxygen, and sulfur,

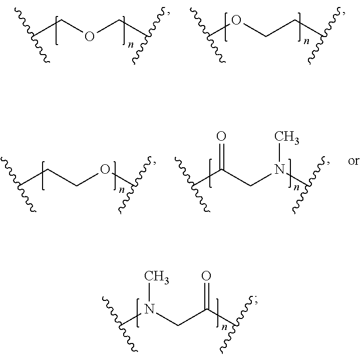

each n is independently 1-10; and each R is independently hydrogen, or C₁₋₆alkyl;
(102) L is a bivalent, saturated, straight hydrocarbon chain, comprising 5-15 carbon atoms; wherein between 2 and 6 alkylene units of L are independently replaced by —O—, —N(R), —S—, —OC(O)—, —C(O)O—, —S(O)—, —S(O)₂—, —N(R)S(O)₂—, —S(O)₂N(R)—, —N(R)C(O)—, —C(O)N(R)—, —OC(O)N(R)—, 5-7 membered saturated heterocyclylenyl having 1-2 heteroatoms independently selected from nitrogen, oxygen, and sulfur,

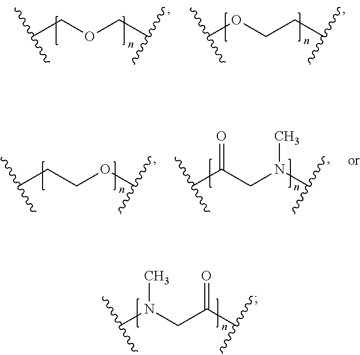

each n is independently 1-5; and each R is independently hydrogen, or C₁₋₃alkyl;
(103) L is a bivalent, saturated, straight hydrocarbon chain, comprising 1-10 carbon atoms; wherein between 0 and 4 alkylene units of L are independently replaced by —O—, —N(R), —S—, —OC(O)—, —C(O)O—, —S(O)—, —S(O)₂—, —N(R)S(O)₂—, —S(O)₂N(R)—, —N(R)C(O)—, —C(O)N(R)—, —OC(O)N(R)—, 5-7 membered saturated heterocyclylenyl having 1-2 heteroatoms independently selected from nitrogen, oxygen, and sulfur,

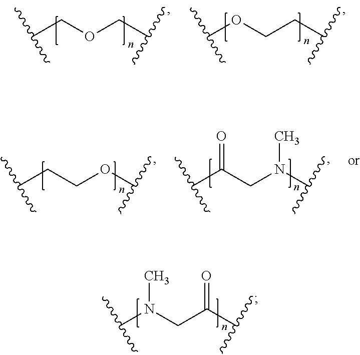

each n is independently 1-5; and each R is independently hydrogen, or C₁₋₃alkyl;
(104) L is a bivalent, saturated, straight or branched hydrocarbon chain comprising 1-10 carbon atoms; wherein between 0 and 4 alkylene units of L are independently replaced by —O—, —N(R), —S—, —OC(O)—, —C(O)O—, —S(O)—, —S(O)₂—, —N(R)S(O)₂—, —S(O)₂N(R)—, —N(R)C(O)—, —C(O)N(R)—, or —OC(O)N(R)—; and each R is independently hydrogen or C₁₋₃alkyl (such as methyl);
(105) L is selected from:

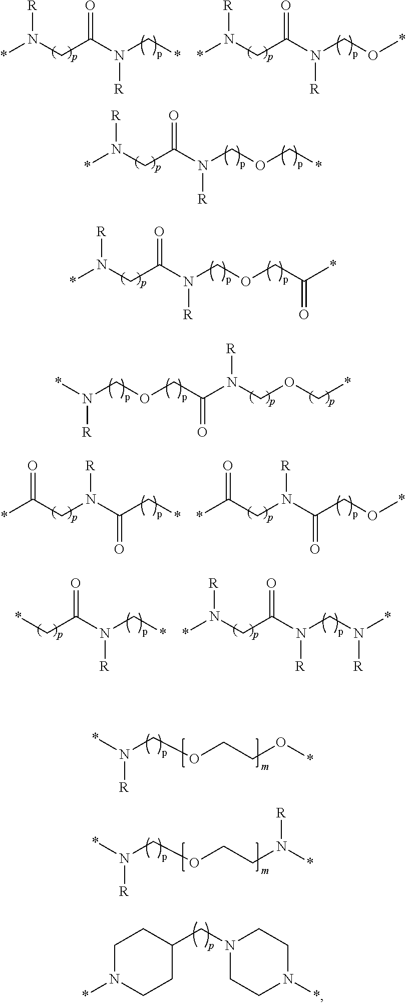

wherein * is the point of attachment to the KBG or the PBG; m is 0 to 4; each p is independently 1 to 4; and each R is independently hydrogen or C₁₋₃alkyl (such as methyl);
(106) L is selected from:

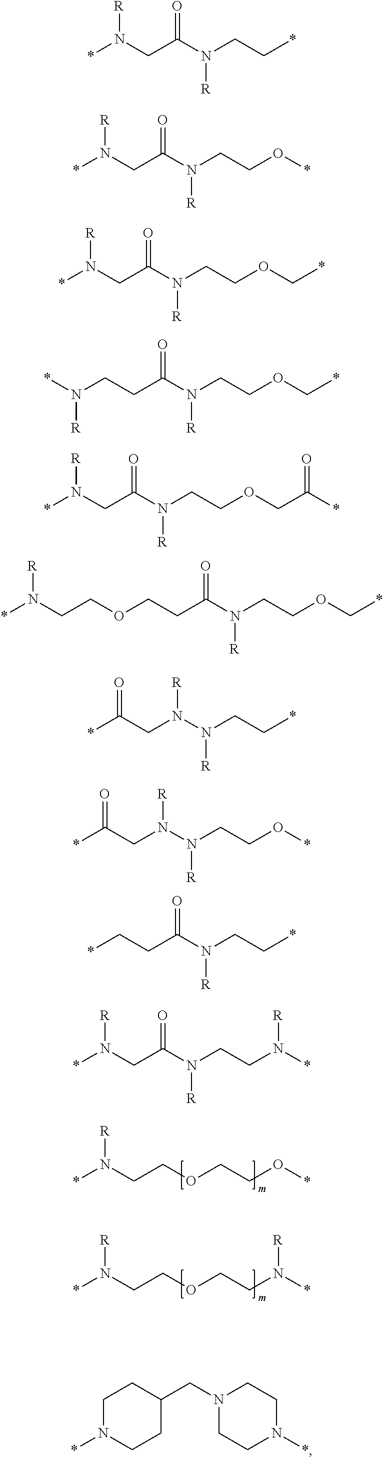

wherein * is the point of attachment to the KBG or the PBG; m is 0 to 4; and each R is independently hydrogen or C₁₋₃alkyl (such as methyl);
(107) L is covalently attached to any one of the groups —R¹, —N(R⁴)(R⁵), or -L¹C(R⁶)(R⁷)L²C(R⁸)(R⁹)(R¹⁰) on the KBG;
(108) L is covalently attached to any one of the groups —R¹, —N(R⁴)(R⁵), or -L¹C(R⁶)(R⁷)L²C(R⁸)(R⁹)(R¹⁰) on the KBG, provided that L is not covalently attached through the R⁸ substituent;
(109) L is covalently attached to the group —R¹ on the KBG;
(110) L is covalently attached to the group —N(R⁴)(R⁵) on the KBG;
(111) L is covalently attached to the group -L¹C(R⁶)(R⁷)L²C(R⁸)(R⁹)(R¹⁰) on the KBG;
(112) L is covalently attached to the group -L¹C(R⁶)(R⁷)L²C(R⁸)(R⁹)(R¹⁰) on the KBG, provided that L is not covalently attached through the R⁸ substituent;
(113) L is covalently attached to the group —R¹ on the KBG, wherein R¹ is a group as described in paragraph (12) above;
(114) L is covalently attached to the group —R¹ on the KBG, wherein R¹ is a group selected from:

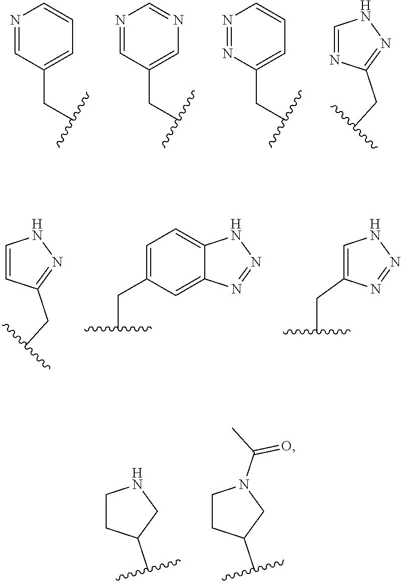

wherein [symbol] represents the point of attachment of the group to the oxygen atom of the rest of the KBG; and each cyclic group is optionally substituted with one or more substituents independently selected from C₁₋₄alkyl, C₁₋₃haloalkyl, C₃₋₇cycloalkyl, C₁₋₄alkylene-R³⁰, halo, OH, C₁₋₃alkoxy, heterocyclyl and cyano;
(115) L is covalently attached to the group —R¹ on the KBG, wherein R¹ is a group as described in paragraph (113) or paragraph (114) above; and wherein L is covalently attached to the heteroaryl ring or heterocyclyl ring in the above R¹ groups, or to a substituent on said heteroaryl or heterocyclyl rings;
(116) L is covalently attached to the group —R¹ on the KBG, wherein R¹ is a group selected from:

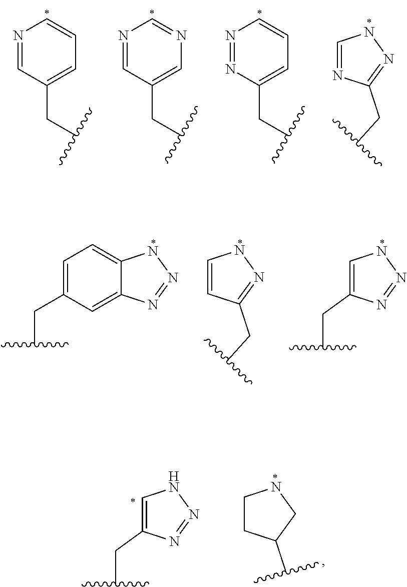

wherein [symbol] represents the point of attachment of the group to the oxygen atom of the rest of the KBG; each group is optionally substituted with one or more substituents independently selected from C₁₋₄alkyl, C₁₋₃haloalkyl, C₃₋₇cycloalkyl, C₁₋₄alkylene-R³⁰, halo, OH, C₁₋₃alkoxy, heterocyclyl and cyano; and wherein * represents the point of attachment of the linking group L;
(117) L is covalently attached to the group —N(R⁴)(R⁵) on the KBG, and replaces one of R4 or R5;
(118) L is covalently attached to the group —N(R⁴)(R⁵) on the KBG, wherein —N(R⁴)(R⁵) is a group as described in paragraph (32) above, and L is covalently attached to any ring atom;
(119) L is covalently attached to the group —N(R⁴)(R⁵) on the KBG, and —N(R⁴)(R⁵) is selected from one of the following structures:

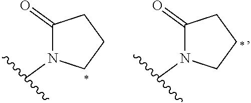

wherein [symbol] represents the point of attachment of the group to the rest of the KBG; each group is optionally substituted with one or more substituents independently selected from C₁₋₄alkyl, halo and OH; and wherein * represents the point of attachment of the linking group L;
(120) L is covalently attached to the group -L¹C(R⁶)(R⁷)L²C(R³)(R⁹)(R¹⁰) on the KBG, and -L¹C(R⁶)(R⁷)L²C(R⁸)(R⁹)(R¹⁰) is selected from one of the following structures:

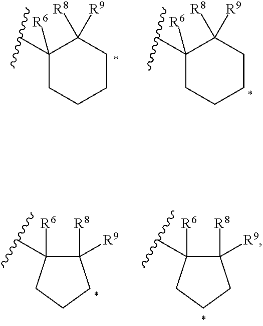

wherein [symbol] represents the point of attachment of the group to the rest of the KBG; each group is optionally substituted with one or more substituents independently selected from C₁₋₄alkyl, halo, OH, C₁₋₃alkoxy and deuterium; and wherein * represents the point of attachment of the linking group L;
(121) L is covalently attached to the group -L¹C(R⁶)(R⁷)L²C(R⁸)(R⁹)(R¹⁰) on the KBG, and -L¹C(R⁶)(R⁷)L²C(R⁸)(R⁹)(R¹⁰) is selected from one of the following structures:

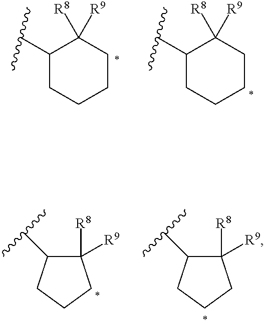

wherein [symbol] represents the point of attachment of the group to the rest of the KBG; each group is optionally substituted with one or more substituents independently selected from methyl and fluoro; and wherein * represents the point of attachment of the linking group L;
(122) L is covalently attached to the group -L¹C(R⁶)(R⁷)L²C(R³)(R⁹)(R¹⁰) on the KBG, and -L¹C(R⁶)(R⁷)L²C(R⁸)(R⁹)(R¹⁰) is selected from one of the following structures:

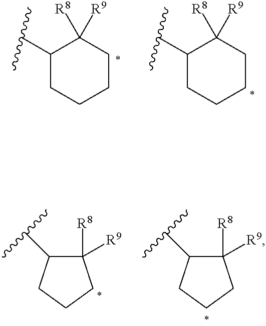

wherein [symbol] represents the point of attachment of the group to the rest of the KBG; R⁸ is selected from CO₂H, C(O)NHSO₂Me and tetrazolyl; R⁹ is hydrogen or methyl; and wherein * represents the point of attachment of the linking group L;
(123) L is covalently attached to the group -L¹C(R⁶)(R⁷)L²C(R⁸)(R⁹)(R¹⁰) on the KBG, and -L¹C(R⁶)(R⁷)L²C(R⁸)(R⁹)(R¹⁰) is selected from one of the following structures:

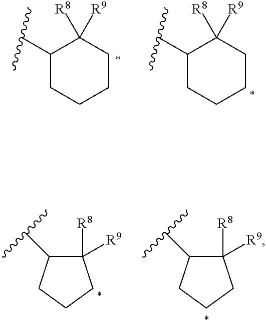

wherein [symbol] represents the point of attachment of the group to the rest of the KBG; R⁸ is CO₂H; R⁹ is methyl; and wherein * represents the point of attachment of the linking group L;
(124) PBG is a protein binding group comprising a moiety having a binding affinity to a target protein of interest, wherein the target protein of interest is:
   (i) a bromodomain and extra-terminal (BET) family protein (such as BRD3, BRD4 or BRD9);
   (ii) an androgen receptor;
   (iii) an estrogen receptor;
   (iv) BCL-XL;
   (v) IRAK4;
   (vi) STAT3;
   (vii) BTK; or
   (viii) TRK;
(125) PBG is a protein binding group comprising a moiety having a binding affinity to a target protein of interest, wherein the target protein of interest is BRD4;
(126) PBG is a protein binding group comprising a moiety having a binding affinity to a target protein of interest, wherein the target protein of interest is BRD4, and the moiety having a binding affinity is:

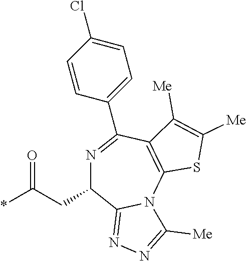 [SMILES: *C(=O)C[C@@H]1N=C(c2ccc(Cl)cc2)c2c(sc(C)c2C)-n2c(C)nnc21]

wherein * represents the point of attachment of the linking group L.

Suitably, R¹ is as defined in any one of paragraphs (1) to (12) above. In an embodiment, R¹ is as defined in paragraph (2) above. In another embodiment, R¹ is as defined in paragraphs (7) to (9) above. In another embodiment, R¹ is as defined in paragraph (12) above.

Suitably, R² is as defined in any one of paragraphs (13) to (14) above. In an embodiment, R² is as defined in paragraph (14) above.

Suitably, R³ is as defined in any one of paragraphs (15) to (19) above. In another embodiment, R³ is as defined in paragraphs (18) to (19) above.

Suitably, R⁴ is as defined in paragraph (20) above.

Suitably, R⁵ is as defined in paragraph (21) above.

Suitably, R⁴ and R⁵ are as defined in any one of paragraphs (22) to (24) above.

In another embodiment, R⁴ and R⁵ are as defined in paragraph (24) above. In an embodiment, R⁴ and R⁵ are as defined in paragraphs (25) to (32) above. In another embodiment, R⁴ and R⁵ are as defined in paragraph (29) above. In a further embodiment, R⁴ and R⁵ are as defined in paragraph (32) above.

Suitably, L¹ and L² are as defined in any one of paragraphs (33) to (36) above. In another embodiment, L¹ and L² are as defined in paragraph (36) above.

Suitably, R⁶ and R⁷ are as defined in any one of paragraphs (37) to (40) above.

Suitably, R⁸ is as defined in any one of paragraphs (41) to (46) above. In another embodiment, R⁸ is as defined in paragraphs (45) to (46) above. In an embodiment, R⁸ is as defined in paragraph (46) above.

Suitably, R^{8a} is as defined in any one of paragraphs (47) to (48) above. In an embodiment, R^{8a} is as defined in paragraph (48) above.

Suitably, R^{8b} is as defined in any one of paragraphs (49) to (54) above. In an embodiment, R^{8b} is as defined in paragraph (53) or (54) above.

Suitably, R^{8a} and R^{8b} are as defined in any one of paragraphs (55) to (61) above.

In an embodiment, R^{8a} and R^{8b} are as defined in paragraph (58) or (61) above.

Suitably, R^{8c} is as defined in any one of paragraphs (62) to (64) above. In an embodiment, R^{8c} is as defined in paragraph (64) above.

Suitably, R⁹ is as defined in any one of paragraphs (65) to (71) above. In an embodiment, R⁹ is as defined in paragraph (71) above.

Suitably, R¹⁰ is as defined in paragraph (72) above.

Suitably, R⁹ and R¹⁰ are as defined in paragraph (73) above.

Suitably, L², R⁷ and R¹⁰ are as defined in any one of paragraphs (74) to (77) above. In another embodiment, L², R⁷ and R¹⁰ are as defined in paragraph (77) above.

Suitably, R¹¹ is as defined in any one of paragraphs (78) to (89) above. In a further embodiment, R¹ is as defined in paragraph (2) above and R¹¹ is as defined in paragraphs (87) to (89) above.

Suitably, R²⁹ is as defined in paragraphs (90) to (91) above. In an embodiment, R²⁹ is as defined in paragraph (91) above.

Suitably, R³⁰ is as defined in paragraphs (92) to (93) above. In an embodiment, R³⁰ is as defined in paragraph (93) above.

Suitably, R⁴⁰ is as defined in paragraph (94) above.

Suitably, R⁴¹ is as defined in paragraph (95) above.

Suitably, R⁴⁰ and R⁴¹ are as defined in any one of paragraphs (96) to (98) above.

In another embodiment, R⁴⁰ and R⁴¹ are as defined in paragraph (98) above.

Suitably, L is as defined in any one of paragraphs (99) to (106) above. In another embodiment, L is as defined in paragraphs (102) to (106) above.

Suitably, L is covalently attached to the KBG as defined in any one of paragraphs (107) to (123) above.

Suitably, the PBG is as defined in any one of paragraphs (124) to (126) above.

In an embodiment, the KBG is selected from one of the structural formulae (II) to (X) shown below:

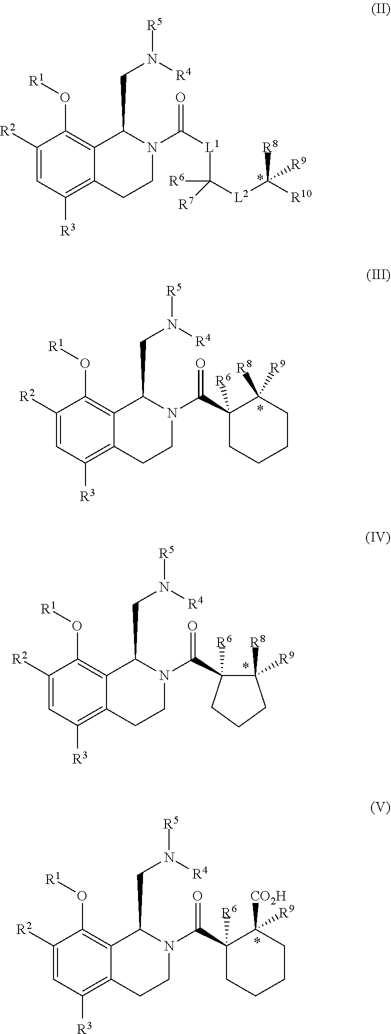

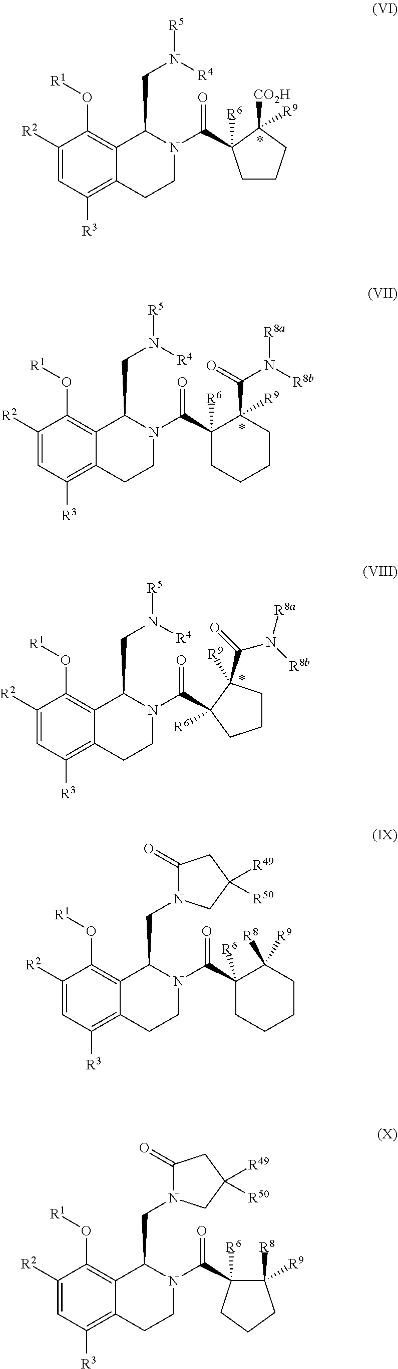

wherein R¹ to R¹⁰, L¹ and L² are as defined herein; the cyclohexyl or cyclopentyl ring in formulae (Ill) to (X) is optionally substituted with one or more substituents independently selected from C₁₋₄alkyl, halo, OH, C₁₋₃alkoxy and deuterium and is optionally bridged by a C₁₋₃alkylene group connecting two carbon atoms of the ring, or R⁹ is optionally a C₁₋₃alkylene group connecting C* to a carbon atom of the ring; and R⁴⁹ and R⁵⁰ are independently selected from hydrogen, C₁₋₄alkyl and halo; or R⁴⁹ and R⁵⁰, together with the carbon atom to which they are attached, form a cyclopropyl or cyclobutyl ring.

In an embodiment, the KEG is selected from one of the structural formulae (XI) to (XVI) shown below:

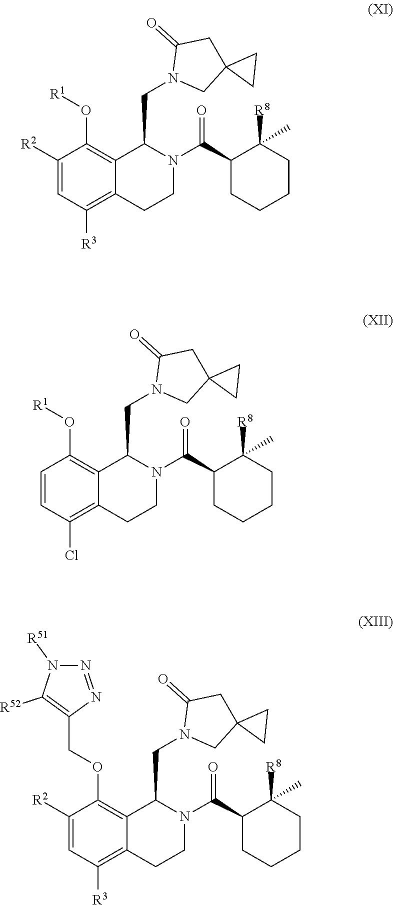

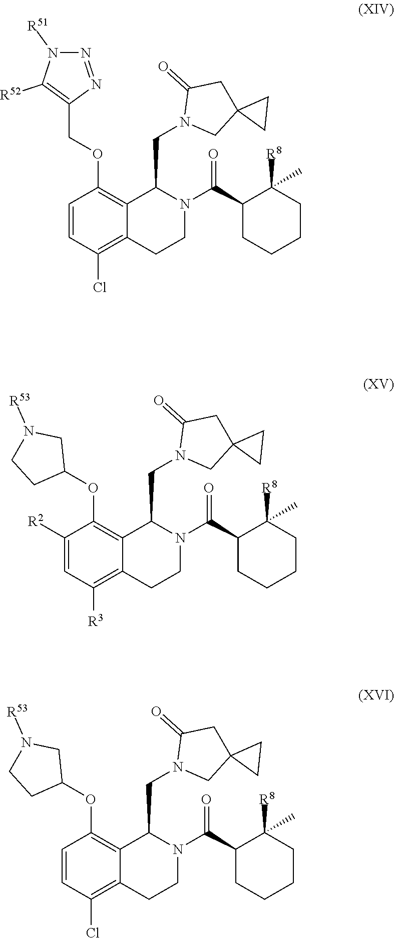

wherein R¹ to R³ are as defined herein; R⁸ is CO₂H or C(O)NR^{8a}R^{8b}; R⁵¹ and R⁵² are independently selected from hydrogen, methyl, fluoromethyl, and difluoromethyl; and R⁵³ is hydrogen, methyl or C(O)Me.

In an embodiment, the KBG is selected from one of the following groups: (1S,2R)-2-((S)-1-((1,3-dioxoisoindolin-2-yl)methyl)-8-(2-(5-methylisoxazole-3-carboxamido)ethoxy)-1,2,3,4-tetrahydroisoquinoline-2-carbonyl)cyclohexane-1-carboxylic acid;
(1S,2R)-2-((S)-8-(2-(Benzo[d]oxazole-2-carboxamido)ethoxy)-1-((1,3-dioxoisoindolin-2-yl)methyl)-1,2,3,4-tetrahydroisoquinoline-2-carbonyl)cyclohexane-1-carboxylic acid;
(1S,2R)-2-((S)-8-(((S)-1-Acetylpyrrolidin-3-yl)oxy)-1-((1,3-dioxoisoindolin-2-yl)methyl)-1,2,3,4-tetrahydroisoquinoline-2-carbonyl)cyclohexane-1-carboxylic acid;
(1S,2R)-2-((S)-1-((1,3-dioxoisoindolin-2-yl)methyl)-8-(((S)-1-(5-methylisoxazole-3-carbonyl)pyrrolidin-3-yl)oxy)-1,2,3,4-tetrahydroisoquinoline-2-carbonyl)cyclohexane-1-carboxylic acid;
(1S,2R)-2-((S)-5-bromo-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)cyclohexane-1-carboxylic acid;
(1S,2R)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)cyclohexane-1-carboxylic acid;
(1S,2R)-2-((S)-1-((1,3-dioxoisoindolin-2-yl)methyl)-8-(((S)-1-(2-methylthiazole-5-carbonyl)pyrrolidin-3-yl)oxy)-1,2,3,4-tetrahydroisoquinoline-2-carbonyl)cyclohexane-1-carboxylic acid;
(1S,2R)-2-((S)-1-((1,3-dioxoisoindolin-2-yl)methyl)-8-((1-methyl-1H-benzo[d][1,2,3]triazol-5-yl)methoxy)-1,2,3,4-tetrahydroisoquinoline-2-carbonyl)cyclohexane-1-carboxylic acid;
(1S,2R)-2-((S)-1-((1,3-dioxoisoindolin-2-yl)methyl)-8-((1-methyl-1H-1,2,3-triazol-4-yl)methoxy)-1,2,3,4-tetrahydroisoquinoline-2-carbonyl)cyclohexane-1-carboxylic acid;
(1S,2R)-2-((S)-1-((1,3-dioxoisoindolin-2-yl)methyl)-8-((1-methyl-1H-benzo[d]imidazol-5-yl)methoxy)-1,2,3,4-tetrahydroisoquinoline-2-carbonyl)cyclohexane-1-carboxylic acid;
(1S,2R)-2-((S)-8-(((S)-1-acetylpyrrolidin-3-yl)oxy)-5-bromo-1-((1,3-dioxoisoindolin-2-yl)methyl)-1,2,3,4-tetrahydroisoquinoline-2-carbonyl)cyclohexane-1-carboxylic acid;
(1S,2R)-2-((S)-8-(((S)-1-acetylpyrrolidin-3-yl)oxy)-5-chloro-1-((1,3-dioxoisoindolin-2-yl)methyl)-1,2,3,4-tetrahydroisoquinoline-2-carbonyl)cyclohexane-1-carboxylic acid;
(1S,2R)-2-((S)-8-(((S)-1-acetylpyrrolidin-3-yl)oxy)-5,7-dichloro-1-((1,3-dioxoisoindolin-2-yl)methyl)-1,2,3,4-tetrahydroisoquinoline-2-carbonyl)cyclohexane-1-carboxylic acid;
(1S,2R)-2-((S)-5-chloro-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)cyclohexane-1-carboxylic acid;
(1S,2R)-2-((S)-1-((1-oxoisoindolin-2-yl)methyl)-8-(((S)-1-(thiazole-5-carbonyl)pyrrolidin-3-yl)oxy)-1,2,3,4-tetrahydroisoquinoline-2-carbonyl)cyclohexane-1-carboxylic acid;
(1S,2R)-2-((S)-8-([1,2,4]triazolo[4,3-a]pyridin-3-ylmethoxy)-5-bromo-1-((1-oxoisoindolin-2-yl)methyl)-1,2,3,4-tetrahydroisoquinoline-2-carbonyl)cyclohexane-1-carboxylic acid;
(1S,2R)-2-((S)-8-((1-isopropyl-1H-1,2,3-triazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-8-((1-methyl-1H-benzo[d][1,2,3]triazol-5-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-8-(((S)-1-acetylpyrrolidin-3-yl)oxy)-5-bromo-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-(((S)-1-(2-methylthiazole-5-carbonyl)pyrrolidin-3-yl)oxy)-1-((1-oxoisoindolin-2-yl)methyl)-1,2,3,4-tetrahydroisoquinoline-2-carbonyl)cyclohexane-1-carboxylic acid;
(1S,2R)-2-((1S)-5-bromo-8-(((3S)-1-(5-methyl-5H-1|4-thiazol-2-yl)pyrrolidin-3-yl)oxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-(((S)-1-(5-methylpyridazin-3-yl)pyrrolidin-3-yl)oxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((4-methyl-4H-1,2,4-triazol-3-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((5-methyl-1,2,4-oxadiazol-3-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-Bromo-8-((1-methyl-1H-imidazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((5-methylisoxazol-3-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((2-ethyl-2H-1,2,3-triazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((1S)-5-bromo-8-((5-methylthiazol-2-yl)methoxy)-1-((1-oxo-1,3,3a,4,5,6-hexahydro-2H-isoindol-2-yl)methyl)-1,2,3,4-tetrahydroisoquinoline-2-carbonyl)cyclohexane-1-carboxylic acid;
(1S,2R)-2-((S)-5-bromo-8-((1-methyl-1H-1,2,4-triazol-5-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-1-((1-oxoisoindolin-2-yl)methyl)-8-(pyridazin-3-ylmethoxy)-1,2,3,4-tetrahydroisoquinoline-2-carbonyl)cyclohexane-1-carboxylic acid;
(1S,2R)-2-((S)-5-bromo-8-((1-(cyclopropylmethyl)-1H-1,2,3-triazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-(imidazo[1,2-a]pyridin-7-ylmethoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((1-methyl-1H-benzo[d]imidazol-5-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((1-methyl-1H-1,2,3-triazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((1-isopropyl-1H-1,2,3-triazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((5-methyl-1,3,4-oxadiazol-2-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((7-methoxy-1-methyl-1H-benzo[d][1,2,3]triazol-5-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((1-methyl-1H-benzo[d][1,2,3]triazol-5-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((1-methyl-1H-1,2,4-triazol-3-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((1-isopropyl-1H-imidazol-2-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((5-(difluoromethyl)-1-methyl-1H-1,2,3-triazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((4-ethyl-4H-1,2,4-triazol-3-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((5-cyano-1-ethyl-1H-imidazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-1-((1-oxoisoindolin-2-yl)methyl)-8-((5,6,7,8-tetrahydro-[1,2,4]triazolo[4,3-a]pyridin-3-yl)methoxy)-1,2,3,4-tetrahydroisoquinoline-2-carbonyl)cyclohexane-1-carboxylic acid;
(1S,2R)-2-((1S)-5-bromo-8-(1-(1-methyl-1H-1,2,3-triazol-4-yl)ethoxy)-1-((1-oxoisoindolin-2-yl)methyl)-1,2,3,4-tetrahydroisoquinoline-2-carbonyl)cyclohexane-1-carboxylic acid;
(1S,2R)-2-((1S)-5-bromo-8-(1-(1-isopropyl-1H-1,2,3-triazol-4-yl)ethoxy)-1-((1-oxoisoindolin-2-yl)methyl)-1,2,3,4-tetrahydroisoquinoline-2-carbonyl)cyclohexane-1-carboxylic acid;
(1S,2R)-2-((1S)-5-bromo-8-(1-(1-isopropyl-1H-1,2,3-triazol-4-yl)ethoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-chloro-8-((1-methyl-1H-benzo[d][1,2,3]triazol-5-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-chloro-8-((5-methylisoxazol-3-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-8-([1,2,4]triazolo[4,3-a]pyridin-3-ylmethoxy)-5-chloro-1-((1-oxoisoindolin-2-yl)methyl)-1,2,3,4-tetrahydroisoquinoline-2-carbonyl)cyclohexane-1-carboxylic acid;
(1S,2R)-2-((S)-5-chloro-1-((1-oxoisoindolin-2-yl)methyl)-8-((1-(2,2,2-trifluoroethyl)-1H-imidazol-2-yl)methoxy)-1,2,3,4-tetrahydroisoquinoline-2-carbonyl)cyclohexane-1-carboxylic acid;
(1S,2R)-2-((S)-5-chloro-8-((1-(2,2-difluoroethyl)-1H-imidazol-2-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-chloro-8-((5-(difluoromethyl)-1-methyl-1H-1,2,3-triazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-chloro-8-((1-methyl-5-(trifluoromethyl)-1H-1,2,3-triazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-chloro-8-((1-ethyl-1H-pyrazol-3-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-chloro-8-((5-(difluoromethyl)-1-methyl-1H-imidazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-chloro-8-((1-isopropyl-1H-1,2,3-triazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-fluoro-8-((1-isopropyl-1H-1,2,3-triazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-8-((1-isopropyl-1H-1,2,3-triazol-4-yl)methoxy)-5-methyl-1-((1-oxoisoindolin-2-yl)methyl)-1,2,3,4-tetrahydroisoquinoline-2-carbonyl)cyclohexane-1-carboxylic acid;
(1S,2R)-2-((S)-5-cyano-8-((1-isopropyl-1H-1,2,3-triazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-8-((1-isopropyl-1H-1,2,3-triazol-4-yl)methoxy)-5-methoxy-1-((1-oxoisoindolin-2-yl)methyl)-1,2,3,4-tetrahydroisoquinoline-2-carbonyl)cyclohexane-1-carboxylic acid;
(1S,2R)-2-((S)-8-((1-isopropyl-1H-1,2,3-triazol-4-yl)methoxy)-1-((1-oxoisoindolin-2-yl)methyl)-5-(trifluoromethyl)-1,2,3,4-tetrahydroisoquinoline-2-carbonyl)cyclohexane-1-carboxylic acid;
(1S,2R)-2-((S)-5-bromo-1-((2-oxopyrrolidin-1-yl)methyl)-8-(((S)-1-(thiazole-5-carbonyl)pyrrolidin-3-yl)oxy)-1,2,3,4-tetrahydroisoquinoline-2-carbonyl)cyclohexane-1-carboxylic acid;
(1S,2R)-2-((S)-8-([1,2,4]triazolo[4,3-a]pyridin-3-ylmethoxy)-5-bromo-1-((2-oxopyrrolidin-1-yl)methyl)-1,2,3,4-tetrahydroisoquinoline-2-carbonyl)cyclohexane-1-carboxylic acid;
(1S,2R)-2-((S)-5-bromo-8-((5-methylthiazol-2-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-5-bromo-8-((5-isopropyl-1,2,4-oxadiazol-3-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-8-(benzo[d]isoxazol-3-ylmethoxy)-5-bromo-1-((2-oxopyrrolidin-1-yl)methyl)-1,2,3,4-tetrahydroisoquinoline-2-carbonyl)cyclohexane-1-carboxylic acid;
(1S,2R)-2-((S)-8-(benzo[d]isothiazol-3-ylmethoxy)-5-chloro-1-((2-oxopyrrolidin-1-yl)methyl)-1,2,3,4-tetrahydroisoquinoline-2-carbonyl)cyclohexane-1-carboxylic acid;
(1S,2R)-2-((S)-5-chloro-1-((2-oxopyrrolidin-1-yl)methyl)-8-(pyridin-2-ylmethoxy)-1,2,3,4-tetrahydroisoquinoline-2-carbonyl)cyclohexane-1-carboxylic acid;
(1S,2R)-2-((S)-5-chloro-8-((5-methylisothiazol-3-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-5-chloro-8-(isothiazol-3-ylmethoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-8-(benzo[d]isoxazol-3-ylmethoxy)-5-chloro-1-((2-oxopyrrolidin-1-yl)methyl)-1,2,3,4-tetrahydroisoquinoline-2-carbonyl)cyclohexane-1-carboxylic acid
(1S,2R)-2-((1S)-5-bromo-8-((1-isopropyl-1H-1,2,3-triazol-4-yl)methoxy)-1-((3-methyl-2-oxopyrrolidin-1-yl)methyl)-1,2,3,4-tetrahydroisoquinoline-2-carbonyl)cyclohexane-1-carboxylic acid;
(1S,2R)-2-((1S)-5-bromo-8-((1-isopropyl-1H-1,2,3-triazol-4-yl)methoxy)-1-((3-methyl-2-oxopyrrolidin-1-yl)methyl)-1,2,3,4-tetrahydroisoquinoline-2-carbonyl)cyclohexane-1-carboxylic acid;
(1S,2R)-2-((S)-5-bromo-8-((1-isopropyl-1H-1,2,3-triazol-4-yl)methoxy)-1-(((R)-4-methyl-2-oxopyrrolidin-1-yl)methyl)-1,2,3,4-tetrahydroisoquinoline-2-carbonyl)cyclohexane-1-carboxylic acid;
(1S,2R)-2-((S)-5-bromo-8-((1-isopropyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)cyclohexane-1-carboxylic acid;
(1S,2R)-2-((S)-5-bromo-8-((1-isopropyl-1H-1,2,3-triazol-4-yl)methoxy)-1-(((S)-4-methyl-2-oxopyrrolidin-1-yl)methyl)-1,2,3,4-tetrahydroisoquinoline-2-carbonyl)cyclohexane-1-carboxylic acid;
(1S,2R)-2-((S)-5-chloro-1-((2-oxopyrrolidin-1-yl)methyl)-8-(pyridin-3-ylmethoxy)-1,2,3,4-tetrahydroisoquinoline-2-carbonyl)cyclohexane-1-carboxylic acid;
(1S,2R)-2-((S)-5-chloro-8-((5-(difluoromethyl)-1-ethyl-1H-1,2,3-triazol-4-yl)methoxy)-1-((2-oxopyrrolidin-1-yl)methyl)-1,2,3,4-tetrahydroisoquinoline-2-carbonyl)cyclohexane-1-carboxylic acid;
(R)-4-((S)-8-(benzo[d]isoxazol-3-ylmethoxy)-5-chloro-1-((2-oxopyrrolidin-1-yl)methyl)-3,4-dihydroisoquinolin-2(1H)-yl)-3-methyl-4-oxobutanoic acid;
1-(((S)-2-((1R,2S)-2-(2H-tetrazol-5-yl)cyclohexane-1-carbonyl)-8-(benzo[d]isoxazol-3-ylmethoxy)-5-chloro-1,2,3,4-tetrahydroisoquinolin-1-yl)methyl)pyrrolidin-2-one;
(1S,2R)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-5-chloro-8-((5-isopropyl-1,2,4-oxadiazol-3-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-5-chloro-8-((5-(difluoromethyl)-1-methyl-1H-1,2,3-triazol-4-yl)methoxy)-1-((4-methyl-1H-pyrazol-1-yl)methyl)-1,2,3,4-tetrahydroisoquinoline-2-carbonyl)cyclohexane-1-carboxylic acid;
(1S,2R)-2-((S)-5-chloro-1-((2-oxopyrrolidin-1-yl)methyl)-8-(pyrimidin-5-ylmethoxy)-1,2,3,4-tetrahydroisoquinoline-2-carbonyl)cyclohexane-1-carboxylic acid;
(1S,2R)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)cyclopentane-1-carboxylic acid;
(1R,2S)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)cyclopentane-1-carboxylic acid;
(1S,2R)-2-((S)-5-chloro-1-((2-oxopyrrolidin-1-yl)methyl)-8-(pyridazin-3-ylmethoxy)-1,2,3,4-tetrahydroisoquinoline-2-carbonyl)cyclohexane-1-carboxylic acid;
(1S,2R)-2-((S)-5-chloro-8-((3-methyl-3H-imidazo[4,5-b]pyridin-6-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-5-chloro-8-((1-methyl-1H-benzo[d][1,2,3]triazol-5-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((1,7-dihydroimidazo[1,2-a]pyrimidin-2-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-5-chloro-8-(isoxazolo[5,4-b]pyridin-3-ylmethoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-8-([1,2,4]triazolo[4,3-a]pyridin-3-ylmethoxy)-5-chloro-1-((2-oxopyrrolidin-1-yl)methyl)-1,2,3,4-tetrahydroisoquinoline-2-carbonyl)cyclohexane-1-carboxylic acid;
(1S,2R)-2-((S)-5-chloro-8-((3-methylisoxazolo[5,4-b]pyridin-6-yl)methoxy)-1-((2-oxopyrrolidin-1-yl)methyl)-1,2,3,4-tetrahydroisoquinoline-2-carbonyl)cyclohexane-1-carboxylic acid;
3-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)bicyclo[2.2.1]heptane-2-carboxylic acid;
3-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)tetrahydrofuran-2-carboxylic acid;
(1S,2R)-2-((S)-5-chloro-8-((1-methyl-1H-indazol-3-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-5-chloro-8-((3-methyl-3H-imidazo[4,5-b]pyridin-5-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-8-(benzo[d]isoxazol-3-ylmethoxy)-5-chloro-1-((2-oxopyrrolidin-1-yl)methyl)-1,2,3,4-tetrahydroisoquinoline-2-carbonyl)-1-methylcyclohexane-1-carboxylic acid;
2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-4,4-difluorocyclopentane-1-carboxylic acid;
(1S,2R)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-1-methylcyclopentane-1-carboxylic acid;
(1S,2R)-2-((S)-5-chloro-8-((4,5-dimethyl-4H-1,2,4-triazol-3-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-5-chloro-8-((1-methyl-1H-1,2,3-triazol-4-yl)methoxy)-1-((2-oxopyrrolidin-1-yl)methyl)-1,2,3,4-tetrahydroisoquinoline-2-carbonyl)cyclohexane-1-carboxylic acid;
(1R,2S)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-1-fluorocyclohexane-1-carboxylic acid;
(1S,2R)-2-((S)-5-chloro-8-((1-methyl-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((1,5-dimethyl-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((1-methyl-5-(trifluoromethyl)-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-methyl-1H-1,2,3-triazol-4-yl)methoxy)-1-(((R)-4-methyl-2-oxopyrrolidin-1-yl)methyl)-1,2,3,4-tetrahydroisoquinoline-2-carbonyl)-1-methylcyclopentane-1-carboxylic acid;
(1S,2R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclopentane-1-carboxylic acid;
(1S,2R)-2-((S)-5-chloro-8-((5-(difluoromethyl)-1-methyl-1H-1,2,3-triazol-4-yl)methoxy)-1-(((R)-4-methyl-2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-methyl-1H-1,2,3-triazol-4-yl)methoxy)-1-(((R)-3-methyl-2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-methyl-1H-1,2,3-triazol-4-yl)methoxy)-1-(((R)-3-methyl-2-oxopyrrolidin-1-yl)methyl)-1,2,3,4-tetrahydroisoquinoline-2-carbonyl)-1-methylcyclopentane-1-carboxylic acid;
(1S,2R)-2-((S)-5-chloro-8-((5-(difluoromethyl)-1-methyl-1H-1,2,3-triazol-4-yl)methoxy)-1-(((S)-3-methyl-2-oxopyrrolidin-1-yl)methyl)-1,2,3,4-tetrahydroisoquinoline-2-carbonyl)-1-methylcyclopentane-1-carboxylic acid;
(1S,2R)-2-((S)-5-chloro-8-((5-(difluoromethyl)-1-methyl-1H-1,2,3-triazol-4-yl)methoxy)-1-(((S)-3-methyl-2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-methyl-1H-1,2,3-triazol-4-yl)methoxy)-1-(((S)-4-methyl-2-oxopyrrolidin-1-yl)methyl)-1,2,3,4-tetrahydroisoquinoline-2-carbonyl)-1-methylcyclopentane-1-carboxylic acid;
(1S,2R)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-1-ethylcyclohexane-1-carboxylic acid;
(1S,2R)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-1-ethylcyclopentane-1-carboxylic acid;
(1S,2R)-2-((S)-5-chloro-8-((5-(difluoromethyl)-1-ethyl-1H-1,2,3-triazol-4-yl)methoxy)-1-((2-oxopyrrolidin-1-yl)methyl)-1,2,3,4-tetrahydroisoquinoline-2-carbonyl)-1-methylcyclopentane-1-carboxylic acid;
(1S,2R)-2-((S)-5-chloro-8-((5-(difluoromethyl)-1-ethyl-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-isopropyl-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-isopropyl-1H-1,2,3-triazol-4-yl)methoxy)-1-((2-oxopyrrolidin-1-yl)methyl)-1,2,3,4-tetrahydroisoquinoline-2-carbonyl)-1-methylcyclopentane-1-carboxylic acid;
(1S,2R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((4,5-dimethylisoxazol-3-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((4-chloro-5-methylisoxazol-3-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((4-methylisoxazol-5-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-(methoxymethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-(2-methoxyethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-cyclopropyl-1-methyl-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-1-(((S)-3-methyl-2-oxopyrrolidin-1-yl)methyl)-8-((1-methyl-5-(trifluoromethyl)-1H-1,2,3-triazol-4-yl)methoxy)-1,2,3,4-tetrahydroisoquinoline-2-carbonyl)-1-methylcyclohexane-1-carboxylic acid;
(1S,2R)-2-((S)-5-chloro-1-(((R)-3-methyl-2-oxopyrrolidin-1-yl)methyl)-8-((1-methyl-5-(trifluoromethyl)-1H-1,2,3-triazol-4-yl)methoxy)-1,2,3,4-tetrahydroisoquinoline-2-carbonyl)-1-methylcyclohexane-1-carboxylic acid;
(1S,2R)-2-((S)-5-chloro-1-(((R)-4-methyl-2-oxopyrrolidin-1-yl)methyl)-8-((1-methyl-5-(trifluoromethyl)-2,3-dihydro-1H-1,2,3-triazol-4-yl)methoxy)-1,2,3,4-tetrahydroisoquinoline-2-carbonyl)-1-methylcyclohexane-1-carboxylic acid;
(1S,2S)-2-((S)-5-chloro-1-(((R)-4-methyl-2-oxopyrrolidin-1-yl)methyl)-8-((1-methyl-5-(trifluoromethyl)-1H-1,2,3-triazol-4-yl)methoxy)-1,2,3,4-tetrahydroisoquinoline-2-carbonyl)-1-methylcyclohexane-1-carboxylic acid;
(1S,2R)-2-((S)-5-chloro-8-((1-cyclopropyl-5-(difluoromethyl)-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((4-(difluoromethyl)pyrimidin-5-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5,5-dimethyl-4,5-dihydroisoxazol-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((4-methylisoxazol-3-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-1-((2-oxopyrrolidin-1-yl)methyl)-8-((4,5,6,7-tetrahydrobenzo[d]isoxazol-3-yl)methoxy)-1,2,3,4-tetrahydroisoquinoline-2-carbonyl)-1-methylcyclohexane-1-carboxylic acid;
(1S,2R)-2-((S)-8-((2,5-bis(difluoromethyl)-2H-1,2,3-triazol-4-yl)methoxy)-5-chloro-1-((6-oxo-5-azaspiro[2.4]heptan-5-yl)methyl)-1,2,3,4-tetrahydroisoquinoline-2-carbonyl)-1-methylcyclohexane-1-carboxylic acid;
2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)tetrahydro-2H-pyran-3-carboxylic acid;
(R)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)bicyclo[2.2.2]octane-1-carboxylic acid;
(1S,2R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic-4,4-d2 acid;
(1S,2R)-2-((S)-5-chloro-8-((1,5-dimethyl-1H-1,2,3-triazol-4-yl)methoxy)-7-fluoro-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-7-fluoro-1-((6-oxo-5-azaspiro[2.4]heptan-5-yl)methyl)-8-((4,5,6,7-tetrahydro-[1,2,3]triazolo[1,5-a]pyridin-3-yl)methoxy)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-(2-methoxyethyl)-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5,6-dihydro-4H-pyrrolo[1,2-c][1,2,3]triazol-3-yl)methoxy)-7-fluoro-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-methyl-1H-1,2,3-triazol-4-yl)methoxy)-7-fluoro-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((1-methyl-2-oxo-1,2-dihydropyridin-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2S)-2-((S)-5-chloro-8-((5-(difluoromethyl)-1-methyl-1H-1,2,3-triazol-4-yl)methoxy)-1-(((R)-4-methyl-2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((1-methyl-5-(trifluoromethyl)-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
1-(((S)-5-chloro-8-((5-(difluoromethyl)-1-methyl-1H-1,2,3-triazol-4-yl)methoxy)-2-((1R,2S)-2-methyl-2-(2H-tetrazol-5-yl)cyclohexane-1-carbonyl)-1,2,3,4-tetrahydroisoquinolin-1-yl)methyl)pyrrolidin-2-one;
(1S,2R)-2-((S)-5-chloro-8-((5-(difluoromethyl)-1-methyl-1H-1,2,3-triazol-4-yl)methoxy)-1-((4,4-dimethyl-2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((1-methyl-5-(trifluoromethyl)-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclopentane-1-carboxylic acid;
(1S,2R)-2-((S)-5-chloro-8-((5-methyl-4-(trifluoromethyl)isoxazol-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-(2,2,2-trifluoroethyl)-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((1,5-dimethyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((4-(difluoromethyl)-5-methylisoxazol-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5-cyclopropyl-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5-(methoxymethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((1-methyl-1H-1,2,3-triazol-4-yl)methoxy)-1-(((R)-4-methyl-2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-(methoxymethyl)-1-methyl-1H-1,2,3-triazol-4-yl)methoxy)-1-(((R)-4-methyl-2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-cyclopropyl-1-methyl-1H-1,2,3-triazol-4-yl)methoxy)-1-(((R)-4-methyl-2-oxopyrrolidin-1-yl)methyl)-1,2,3,4-tetrahydroisoquinoline-2-carbonyl)-1-methylcyclohexane-1-carboxylic acid;
(1S,2R)-2-((1S)-5-chloro-8-((7-fluoro-2,7a-dihydrobenzo[d]isoxazol-3-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((6,7-difluorobenzo[d]isoxazol-3-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-methylisoxazol-3-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((3-methyl-1,2,4-oxadiazol-5-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-methyl-1,2,4-oxadiazol-3-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2S)-2-((S)-5-chloro-8-((1-methyl-5-(trifluoromethyl)-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-1-((6-oxo-5-azaspiro[2.4]heptan-5-yl)methyl)-8-((4,5,6,7-tetrahydro-[1,2,3]triazolo[1,5-a]pyridin-3-yl)methoxy)-1,2,3,4-tetrahydroisoquinoline-2-carbonyl)-1-methylcyclohexane-1-carboxylic acid;
(1S,2R)-2-((S)-5-chloro-1-((6-oxo-5-azaspiro[2.4]heptan-5-yl)methyl)-8-((4-(trifluoromethyl)pyrimidin-5-yl)methoxy)-1,2,3,4-tetrahydroisoquinoline-2-carbonyl)-1-methylcyclohexane-1-carboxylic acid;
(1S,2R)-2-((S)-5-chloro-8-((1-cyclopropyl-5-(difluoromethyl)-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((1-(2-(dimethylamino)ethyl)-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-1-methyl-2-((S)-5-methyl-8-((1-methyl-5-(trifluoromethyl)-1H-1,2,3-triazol-4-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-5-chloro-8-((4-methyl-4H-1,2,4-triazol-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5,6-dihydro-8H-[1,2,4]triazolo[3,4-c][1,4]oxazin-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((4-cyclopropyl-5-methyl-4H-1,2,4-triazol-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-fluoro-8-((1-methyl-5-(trifluoromethyl)-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((4,5-dimethyl-4H-1,2,4-triazol-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-1-((6-oxo-5-azaspiro[2.4]heptan-5-yl)methyl)-8-((4,5,6,7-tetrahydro-[1,2,3]triazolo[1,5-a]pyridin-3-yl)methoxy)-1,2,3,4-tetrahydroisoquinoline-2-carbonyl)-1-methylcyclopentane-1-carboxylic acid;
(1S,2R)-2-((S)-5-chloro-8-((5,6-dihydro-4H-pyrrolo[1,2-c][1,2,3]triazol-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-fluoro-1-((6-oxo-5-azaspiro[2.4]heptan-5-yl)methyl)-8-((4,5,6,7-tetrahydro-[1,2,3]triazolo[1,5-a]pyridin-3-yl)methoxy)-1,2,3,4-tetrahydroisoquinoline-2-carbonyl)-1-methylcyclohexane-1-carboxylic acid;
(1S,2R)-2-((S)-5-chloro-8-((6,7-dihydro-5H-pyrrolo[2,1-c][1,2,4]triazol-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5-cyclopropyl-4-methyl-4H-1,2,4-triazol-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((1-methyl-1H-imidazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-1-((6-oxo-5-azaspiro[2.4]heptan-5-yl)methyl)-8-((5,6,7,8-tetrahydro-[1,2,4]triazolo[4,3-a]pyridin-3-yl)methoxy)-1,2,3,4-tetrahydroisoquinoline-2-carbonyl)-1-methylcyclohexane-1-carboxylic acid;
(1S,2R)-2-((S)-5-chloro-8-(((S)-1-(methylsulfonyl)pyrrolidin-3-yl)oxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-8-(((S)-1-acetylpyrrolidin-3-yl)oxy)-5-chloro-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((6,7-dihydro-4H-[1,2,3]triazolo[5,1-c][1,4]oxazin-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
((1S,2R)-1-methyl-2-((S)-8-((1-methyl-5-(trifluoromethyl)-1H-1,2,3-triazol-4-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-5-bromo-8-((1-methyl-5-(trifluoromethyl)-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-(oxetan-3-yl)-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-8-((1,5-bis(difluoromethyl)-1H-1,2,3-triazol-4-yl)methoxy)-5-chloro-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-methyl-1H-1,2,3-triazol-4-yl)methoxy)-1-(((R)-2-methyl-5-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-methyl-1H-1,2,3-triazol-4-yl)methoxy)-1-(((S)-2-methyl-5-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-1-((3-oxomorpholino)methyl)-8-((4,5,6,7-tetrahydro-[1,2,3]triazolo[1,5-a]pyridin-3-yl)methoxy)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)cyclohexane-1-carboxylic acid;
(1S,2R)-2-((S)-5-bromo-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((1-methyl-5-(oxetan-3-yl)-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-methyl-1H-1,2,3-triazol-4-yl)methoxy)-1-((3-oxomorpholino)methyl)-1,2,3,4-tetrahydroisoquinoline-2-carbonyl)-1-methylcyclohexane-1-carboxylic acid;
(1S,6R)-6-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohex-3-ene-1-carboxylic acid;
5-(((S)-5-chloro-8-((5-(difluoromethyl)-1-methyl-1H-1,2,3-triazol-4-yl)methoxy)-2-((1R,2S)-2-(2,3-dihydro-1|2-tetrazol-5-yl)cyclohexane-1-carbonyl)-1,2,3,4-tetrahydroisoquinolin-1-yl)methyl)-5-azaspiro[2.4]heptan-6-one;
(1S,2R)-2-((S)-5-chloro-8-((5-(difluoromethyl)-1-methyl-1H-1,2,3-triazol-4-yl)methoxy)-7-fluoro-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)thiazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5-methylthiazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((2-methyl-2H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1R,3S,4R,6S)-4-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-3-methylbicyclo[4.1.0]heptane-3-carboxylic acid;
(1S,3S,4R,6R)-4-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-3-methylbicyclo[4.1.0]heptane-3-carboxylic acid;
(1S,2R)-2-((1S)-5-chloro-8-((6,7-dihydro-5H-pyrrolo[2,1-c][1,2,4]triazol-7-yl)oxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R,4S,5R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic-4,5-d2 acid;
(1S,2R)-2-((S)-5-chloro-8-((1-methyl-2-oxo-1,2-dihydropyridin-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((1S)-5-chloro-8-((5-(difluoromethyl)-1-methyl-1H-1,2,3-triazol-4-yl)methoxy)-1-((2-oxo-4-(trifluoromethyl)pyrrolidin-1-yl)methyl)-1,2,3,4-tetrahydroisoquinoline-2-carbonyl)-1-methylcyclohexane-1-carboxylic acid;
(1S,2R)-2-((1S)-5-chloro-8-((5-(difluoromethyl)-1-methyl-1H-1,2,3-triazol-4-yl)methoxy)-1-((2-oxo-4-(trifluoromethyl)pyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((1-methyl-6-oxo-1,6-dihydropyridin-2-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((1S)-5-chloro-8-((1-methyl-1,4,5,6-tetrahydrocyclopenta[d][1,2,3]triazol-5-yl)oxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((1S)-5-chloro-8-((1-methyl-1,4,5,6-tetrahydrocyclopenta[d][1,2,3]triazol-5-yl)oxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-4,4-difluoro-1-methylcyclohexane-1-carboxylic acid;
(1S,2R,4S)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-4-fluoro-1-methylcyclohexane-1-carboxylic acid;
(1S,2R,4R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic-4-d acid;
(1S,2R,4S)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-4-hydroxy-1-methylcyclohexane-1-carboxylic acid;
(1S,2R,5S)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-5-hydroxy-1-methylcyclohexane-1-carboxylic acid;
(1S,2R,5R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-5-hydroxy-1-methylcyclohexane-1-carboxylic acid;
(1S,2R,4R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-4-hydroxy-1-methylcyclohexane-1-carboxylic acid;
(2S,3R)-3-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)tetrahydro-2H-pyran-2-carboxylic acid;
(1R,2R,6S)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-6-hydroxy-1-methylcyclohexane-1-carboxylic acid;
(1R,2R,6R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-6-hydroxy-1-methylcyclohexane-1-carboxylic acid;
(1S,2S,3R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-3-hydroxy-1-methylcyclohexane-1-carboxylic acid;
(1S,2S,3S)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-3-hydroxy-1-methylcyclohexane-1-carboxylic acid;
5-(((S)-5-chloro-8-((5-(difluoromethyl)-1-methyl-1H-1,2,3-triazol-4-yl)methoxy)-7-fluoro-2-((1R,2S)-2-methyl-2-(1H-tetrazol-5-yl)cyclohexane-1-carbonyl)-1,2,3,4-tetrahydroisoquinolin-1-yl)methyl)-5-azaspiro[2.4]heptan-6-one;
(1S,2R)-2-((S)-5-chloro-8-((5-(difluoromethyl)-1-methyl-1H-1,2,3-triazol-4-yl)methoxy)-7-fluoro-1-((3-oxomorpholino)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-methyl-1H-1,2,3-triazol-4-yl)methoxy)-7-fluoro-1-(((R)-4-methyl-2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-7-fluoro-1-((6-oxo-5-azaspiro[2.4]heptan-5-yl)methyl)-8-((4,5,6,7-tetrahydro-[1,2,3]triazolo[1,5-a]pyridin-3-yl)methoxy)-1,2,3,4-tetrahydroisoquinoline-2-carbonyl)-1-methylcyclopentane-1-carboxylic acid;
(1S,2R)-2-((S)-5-chloro-8-((4,5-dimethyl-4H-1,2,4-triazol-3-yl)methoxy)-7-fluoro-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-7-fluoro-8-((1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-7-fluoro-8-((5-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-1-((1,3-dioxoisoindolin-2-yl)methyl)-8-(4-(methylamino)-4-oxobutoxy)-1,2,3,4-tetrahydroisoquinoline-2-carbonyl)-N-methylcyclohexane-1-carboxamide;
N-(2-(((S)-1-((1,3-dioxoisoindolin-2-yl)methyl)-2-((1R,2S)-2-(ethylcarbamoyl)cyclohexane-1-carbonyl)-1,2,3,4-tetrahydroisoquinolin-8-yl)oxy)ethyl)-5-methylisoxazole-3-carboxamide;
(1S,2R)-2-((S)-1-(cyclopropanecarboxamidomethyl)-8-(((S)-1-(thiazole-5-carbonyl)pyrrolidin-3-yl)oxy)-1,2,3,4-tetrahydroisoquinoline-2-carbonyl)-N-methylcyclohexane-1-carboxamide;
(1S,2R)-2-((S)-8-((1H-pyrazol-5-yl)methoxy)-1-((1,3-dioxoisoindolin-2-yl)methyl)-1,2,3,4-tetrahydroisoquinoline-2-carbonyl)-N-methylcyclohexane-1-carboxamide;
(1S,2R)-2-((S)-1-(acetamidomethyl)-8-(((S)-1-(thiazole-5-carbonyl)pyrrolidin-3-yl)oxy)-1,2,3,4-tetrahydroisoquinoline-2-carbonyl)-N-methylcyclohexane-1-carboxamide;
(1S,2R)—N-methyl-2-((S)-1-((2-oxooxazolidin-3-yl)methyl)-8-(((S)-1-(thiazole-5-carbonyl)pyrrolidin-3-yl)oxy)-1,2,3,4-tetrahydroisoquinoline-2-carbonyl)cyclohexane-1-carboxamide;
4-(((S)-1-((1,3-dioxoisoindolin-2-yl)methyl)-2-((1R,2S)-2-(methylcarbamoyl)cyclohexane-1-carbonyl)-1,2,3,4-tetrahydroisoquinolin-8-yl)oxy)butanoic acid;
(R)-1-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)-N-methylpiperidine-2-carboxamide;
(S)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-N-methylpyrrolidine-1-carboxamide;
1-(((S)-5-chloro-2-((S)-1-(2,2-difluoroacetyl)piperidine-2-carbonyl)-8-((5-(difluoromethyl)-1-methyl-1H-1,2,3-triazol-4-yl)methoxy)-1,2,3,4-tetrahydroisoquinolin-1-yl)methyl)pyrrolidin-2-one;
(S)-6-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-N-methyl-5-azaspiro[2.4]heptane-5-carboxamide;
(1R,2S)-2-((R)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-N,1-dimethylcyclohexane-1-carboxamide;
(1S,2R)-2-((S)-1-((1,3-dioxoisoindolin-2-yl)methyl)-8-(4-oxo-4-(pyrrolidin-1-yl)butoxy)-1,2,3,4-tetrahydroisoquinoline-2-carbonyl)cyclohexane-1-carboxamide;
5-cyclopropyl-N-(2-(((S)-1-((1,3-dioxoisoindolin-2-yl)methyl)-2-((1R,2S)-2-(methylcarbamoyl)cyclohexane-1-carbonyl)-1,2,3,4-tetrahydroisoquinolin-8-yl)oxy)ethyl)isoxazole-3-carboxamide;
N-(2-(((S)-1-((1,3-dioxoisoindolin-2-yl)methyl)-2-((1R,2S)-2-(methylcarbamoyl)cyclohexane-1-carbonyl)-1,2,3,4-tetrahydroisoquinolin-8-yl)oxy)ethyl)-4,5-dimethylisoxazole-3-carboxamide;
(1S,2R)-2-((S)-8-(((S)-1-acetylpyrrolidin-3-yl)oxy)-1-((1,3-dioxoisoindolin-2-yl)methyl)-1,2,3,4-tetrahydroisoquinoline-2-carbonyl)-N-methylcyclohexane-1-carboxamide;
N-(2-(((S)-2-((1R,2S)-2-(cyclopropylcarbamoyl)cyclohexane-1-carbonyl)-1-((1,3-dioxoisoindolin-2-yl)methyl)-1,2,3,4-tetrahydroisoquinolin-8-yl)oxy)ethyl)-5-methylisoxazole-3-carboxamide;
N-(2-(((S)-1-((1,3-dioxoisoindolin-2-yl)methyl)-2-((1R,2S)-2-(3-hydroxyazetidine-1-carbonyl)cyclohexane-1-carbonyl)-1,2,3,4-tetrahydroisoquinolin-8-yl)oxy)ethyl)-5-methylisoxazole-3-carboxamide;
N-(2-(((S)-2-((1R,2S)-2-(cyclobutylcarbamoyl)cyclohexane-1-carbonyl)-1-((1,3-dioxoisoindolin-2-yl)methyl)-1,2,3,4-tetrahydroisoquinolin-8-yl)oxy)ethyl)-5-methylisoxazole-3-carboxamide;
5-cyclopropyl-N-(2-(((S)-1-((1,3-dioxoisoindolin-2-yl)methyl)-2-((1R,2S)-2-(methylcarbamoyl)cyclohexane-1-carbonyl)-1,2,3,4-tetrahydroisoquinolin-8-yl)oxy)ethyl)isoxazole-3-carboxamide;
N-(2-(((S)-1-((1,3-dioxoisoindolin-2-yl)methyl)-2-((1R,2S)-2-(methylcarbamoyl)cyclohexane-1-carbonyl)-1,2,3,4-tetrahydroisoquinolin-8-yl)oxy)ethyl)benzo[d]oxazole-2-carboxamide;
N-(2-(((S)-1-((1,3-dioxoisoindolin-2-yl)methyl)-2-((1R,2S)-2-(methylcarbamoyl)cyclohexane-1-carbonyl)-1,2,3,4-tetrahydroisoquinolin-8-yl)oxy)ethyl)benzo[d]isoxazole-3-carboxamide;
(1S,2R)-2-((S)-1-((1,3-dioxoisoindolin-2-yl)methyl)-8-(2-((5-methylisoxazole)-4-sulfonamido)ethoxy)-1,2,3,4-tetrahydroisoquinoline-2-carbonyl)-N-methylcyclohexane-1-carboxamide;
(1S,2R)-2-((S)-1-((1,3-dioxoisoindolin-2-yl)methyl)-8-(((S)-1-(pyrimidine-5-carbonyl)pyrrolidin-3-yl)oxy)-1,2,3,4-tetrahydroisoquinoline-2-carbonyl)-N-methylcyclohexane-1-carboxamide;
(1S,2R)-2-((S)-1-((1,3-dioxoisoindolin-2-yl)methyl)-8-(((S)-1-nicotinoylpyrrolidin-3-yl)oxy)-1,2,3,4-tetrahydroisoquinoline-2-carbonyl)-N-methylcyclohexane-1-carboxamide;
(1S,2R)-2-((S)-1-((1,3-dioxoisoindolin-2-yl)methyl)-8-(((S)-1-((5-methylisoxazol-4-yl)sulfonyl)pyrrolidin-3-yl)oxy)-1,2,3,4-tetrahydroisoquinoline-2-carbonyl)-N-methylcyclohexane-1-carboxamide;
(1S,2R)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)-N-methylcyclohexane-1-carboxamide;
N-(2-(((S)-1-((1,3-dioxoisoindolin-2-yl)methyl)-2-((1R,2S)-2-(methylcarbamoyl)cyclohexane-1-carbonyl)-1,2,3,4-tetrahydroisoquinolin-8-yl)oxy)ethyl)thiazole-2-carboxamide;
(1S,2R)-2-((S)-8-(((S)-1-(cyclopropanecarbonyl)pyrrolidin-3-yl)oxy)-1-((1,3-dioxoisoindolin-2-yl)methyl)-1,2,3,4-tetrahydroisoquinoline-2-carbonyl)-N-methylcyclohexane-1-carboxamide;
(1S,2R)-2-((S)-1-((1,3-dioxoisoindolin-2-yl)methyl)-8-(((S)-1-(5-methylisoxazole-3-carbonyl)pyrrolidin-3-yl)oxy)-1,2,3,4-tetrahydroisoquinoline-2-carbonyl)-N-methylcyclohexane-1-carboxamide;
(1S,2R)-2-((S)-1-((1,3-dioxoisoindolin-2-yl)methyl)-8-(oxazol-5-ylmethoxy)-1,2,3,4-tetrahydroisoquinoline-2-carbonyl)-N-methylcyclohexane-1-carboxamide;
(1S,2R)-2-((S)-1-((1,3-dioxoisoindolin-2-yl)methyl)-8-(((S)-1-(2-methylthiazole-5-carbonyl)pyrrolidin-3-yl)oxy)-1,2,3,4-tetrahydroisoquinoline-2-carbonyl)-N-methylcyclohexane-1-carboxamide;
(1S,2R)-2-((S)-8-(((S)-1-(2,4-dimethylthiazole-5-carbonyl)pyrrolidin-3-yl)oxy)-1-((1,3-dioxoisoindolin-2-yl)methyl)-1,2,3,4-tetrahydroisoquinoline-2-carbonyl)-N-methylcyclohexane-1-carboxamide;
(1S,2R)-2-((S)-1-((1,3-dioxoisoindolin-2-yl)methyl)-8-(((S)-1-(2-methylthiazole-4-carbonyl)pyrrolidin-3-yl)oxy)-1,2,3,4-tetrahydroisoquinoline-2-carbonyl)-N-methylcyclohexane-1-carboxamide;
(R)-4-((S)-1-((1,3-dioxoisoindolin-2-yl)methyl)-8-(((S)-1-(thiazole-5-carbonyl)pyrrolidin-3-yl)oxy)-3,4-dihydroisoquinolin-2(1H)-yl)-N,3-dimethyl-4-oxobutanamide;
(1S,2R)—N-methyl-2-((S)-1-((2-oxopyrrolidin-1-yl)methyl)-8-(((S)-1-(thiazole-5-carbonyl)pyrrolidin-3-yl)oxy)-1,2,3,4-tetrahydroisoquinoline-2-carbonyl)cyclohexane-1-carboxamide;
(1S,2R)-2-((S)-1-((1,3-dioxoisoindolin-2-yl)methyl)-8-(pyrazin-2-ylmethoxy)-1,2,3,4-tetrahydroisoquinoline-2-carbonyl)-N-methylcyclohexane-1-carboxamide;
(S)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)-N-methylpiperidine-1-carboxamide;
(1S,2R)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)-N-propylcyclohexane-1-carboxamide;
(1S,2R)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)-N-(2-methoxyethyl)cyclohexane-1-carboxamide;
(1S,2R)—N-(2,2-difluoroethyl)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)cyclohexane-1-carboxamide;
(1S,2R)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)-N-(2,2,2-trifluoroethyl)cyclohexane-1-carboxamide;
(1S,2R)—N-(cyanomethyl)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)cyclohexane-1-carboxamide;
(1S,2R)—N-(2,2-difluoro-3-hydroxypropyl)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)cyclohexane-1-carboxamide;
(1S,2R)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)-N-(2-hydroxyethyl)cyclohexane-1-carboxamide;
1-((S)-1-((S)-1-((1,3-dioxoisoindolin-2-yl)methyl)-8-(((S)-1-(thiazole-5-carbonyl)pyrrolidin-3-yl)oxy)-3,4-dihydroisoquinolin-2(1H)-yl)-1-oxopropan-2-yl)-3-methylurea;
(1S,2R)-2-((S)-8-(((S)-1-acetylpyrrolidin-3-yl)oxy)-5-chloro-1-((1,3-dioxoisoindolin-2-yl)methyl)-1,2,3,4-tetrahydroisoquinoline-2-carbonyl)-N-methylcyclohexane-1-carboxamide;
5-((S)-1-((1,3-dioxoisoindolin-2-yl)methyl)-8-(((S)-1-(thiazole-5-carbonyl)pyrrolidin-3-yl)oxy)-3,4-dihydroisoquinolin-2(1H)-yl)-N-methyl-5-oxopentanamide:
4-((S)-1-((1,3-dioxoisoindolin-2-yl)methyl)-8-(((S)-1-(thiazole-5-carbonyl)pyrrolidin-3-yl)oxy)-3,4-dihydroisoquinolin-2(1H)-yl)-N-methyl-4-oxobutanamide;
(1S,2R)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)-N-ethylcyclohexane-1-carboxamide;
(1S,2R)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)-N-(3-hydroxypropyl)cyclohexane-1-carboxamide;
(1S,2R)—N-methyl-2-((S)-1-(propionamidomethyl)-8-(((S)-1-(thiazole-5-carbonyl)pyrrolidin-3-yl)oxy)-1,2,3,4-tetrahydroisoquinoline-2-carbonyl)cyclohexane-1-carboxamide;
(1S,2R)-2-((S)-1-((1,3-dioxoisoindolin-2-yl)methyl)-8-((1-methyl-1H-pyrazol-4-yl)methoxy)-1,2,3,4-tetrahydroisoquinoline-2-carbonyl)-N-methylcyclohexane-1-carboxamide;
(1S,2R)-2-((S)-8-(((S)-1-acetylpyrrolidin-3-yl)oxy)-5-bromo-1-((1,3-dioxoisoindolin-2-yl)methyl)-1,2,3,4-tetrahydroisoquinoline-2-carbonyl)-N-methylcyclohexane-1-carboxamide;
methyl (S)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)pyrrolidine-1-carboxylate;
(1S,2R)-2-((S)-8-(benzo[d]isoxazol-3-ylmethoxy)-5-chloro-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-8-(benzo[d]isoxazol-3-ylmethoxy)-5-chloro-1-((2-oxopyrrolidin-1-yl)methyl)-1,2,3,4-tetrahydroisoquinoline-2-carbonyl)-N-ethylcyclohexane-1-carboxamide;
(1S,2R)-2-((S)-8-(benzo[d]isoxazol-3-ylmethoxy)-5-chloro-1-((2-oxopyrrolidin-1-yl)methyl)-1,2,3,4-tetrahydroisoquinoline-2-carbonyl)cyclohexane-1-carboxamide;
(1S,2R)-2-((S)-5-bromo-8-((5-isopropyl-1,2,4-oxadiazol-3-yl)methoxy)-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-5-bromo-8-((5-(difluoromethyl)-1-methyl-1H-1,2,3-triazol-4-yl)methoxy)-1-((1-oxoisoindolin-2-yl)methyl)-1,2,3,4-tetrahydroisoquinoline-2-carbonyl)-N-methylcyclohexane-1-carboxamide;
(1S,2R)-2-((S)-5-chloro-8-((5-isopropyl-1,2,4-oxadiazol-3-yl)methoxy)-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-N-methylcyclohexane-1-carboxamide;
(2R,3R)-4-((S)-5-chloro-8-((5-isopropyl-1,2,4-oxadiazol-3-yl)methoxy)-1-((2-oxopyrrolidin-1-yl)methyl)-3,4-dihydroisoquinolin-2(1H)-yl)-N,2,3-trimethyl-4-oxobutanamide;
(1S,2R)-2-((S)-5-chloro-1-((2-oxopyrrolidin-1-yl)methyl)-8-(pyridin-3-ylmethoxy)-1,2,3,4-tetrahydroisoquinoline-2-carbonyl)-N-methylcyclohexane-1-carboxamide;
(1S,2R)-2-((S)-5-chloro-1-((2-oxopyrrolidin-1-yl)methyl)-8-(pyridazin-3-ylmethoxy)-1,2,3,4-tetrahydroisoquinoline-2-carbonyl)-N-methylcyclohexane-1-carboxamide;
(1S,2R)-2-((S)-5-chloro-1-((2-oxopyrrolidin-1-yl)methyl)-8-(pyrazin-2-ylmethoxy)-1,2,3,4-tetrahydroisoquinoline-2-carbonyl)-N-methylcyclohexane-1-carboxamide;
(1S,2R)-2-((S)-5-chloro-1-((2-oxopyrrolidin-1-yl)methyl)-8-(pyridin-2-ylmethoxy)-1,2,3,4-tetrahydroisoquinoline-2-carbonyl)-N-methylcyclohexane-1-carboxamide;
(1S,2R)-2-((S)-5-chloro-1-((2-oxopyrrolidin-1-yl)methyl)-8-(pyrimidin-5-ylmethoxy)-1,2,3,4-tetrahydroisoquinoline-2-carbonyl)-N-methylcyclohexane-1-carboxamide;
2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-N-methylcyclopentane-1-carboxamide;
(1S,2R)-2-((S)-5-chloro-8-(2-(1-methyl-1H-1,2,3-triazol-4-yl)ethoxy)-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-5-chloro-8-((3-methyl-3H-imidazo[4,5-b]pyridin-6-yl)methoxy)-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-N,1-dimethylcyclohexane-1-carboxamide;
(1S,2R)-2-((S)-5-chloro-8-(isoxazolo[5,4-b]pyridin-3-ylmethoxy)-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-5-chloro-8-((3-methylisoxazolo[5,4-b]pyridin-6-yl)methoxy)-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-5-chloro-8-((1-methyl-1H-benzo[d][1,2,3]triazol-5-yl)methoxy)-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-8-([1,2,4]triazolo[4,3-a]pyridin-3-ylmethoxy)-5-chloro-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-5-chloro-8-((1-methyl-1H-1,2,3-triazol-4-yl)methoxy)-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-5-chloro-8-(imidazo[1,2-a]pyrimidin-2-ylmethoxy)-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-5-chloro-8-((5-(difluoromethyl)-1-methyl-1H-1,2,3-triazol-4-yl)methoxy)-1-((1-oxoisoindolin-2-yl)methyl)-1,2,3,4-tetrahydroisoquinoline-2-carbonyl)-N-methylcyclohexane-1-carboxamide;
(1S,2R)-2-((S)-8-([1,2,4]triazolo[4,3-a]pyridin-3-ylmethoxy)-5-chloro-1-((1-oxoisoindolin-2-yl)methyl)-1,2,3,4-tetrahydroisoquinoline-2-carbonyl)-N-methylcyclohexane-1-carboxamide;
(1S,2R)-2-((S)-8-(benzo[d]isoxazol-3-ylmethoxy)-5-chloro-1-((2-oxopyrrolidin-1-yl)methyl)-1,2,3,4-tetrahydroisoquinoline-2-carbonyl)-N-(2,2-difluoro-3-hydroxypropyl)cyclohexane-1-carboxamide;
(S)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-N-methylpiperidine-1-carboxamide;
(1S,2R)-2-((S)-5-chloro-8-((3-methyl-3H-imidazo[4,5-b]pyridin-5-yl)methoxy)-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-5-chloro-8-((1-methyl-1H-indazol-3-yl)methoxy)-1-((2-oxopyrrolidin-1-yl)methyl)-1,2,3,4-tetrahydroisoquinoline-2-carbonyl)-N-methylcyclohexane-1-carboxamide;
3-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-N-methyltetrahydrofuran-2-carboxamide;
(1S,2R)-2-((S)-8-(benzo[d]isoxazol-3-ylmethoxy)-5-chloro-1-((2-oxopyrrolidin-1-yl)methyl)-1,2,3,4-tetrahydroisoquinoline-2-carbonyl)-N,1-dimethylcyclohexane-1-carboxamide;
(1S,2R)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-N,1-dimethylcyclopentane-1-carboxamide;
(1S,2R)-2-((S)-5-chloro-8-((4,5-dimethyl-4H-1,2,4-triazol-3-yl)methoxy)-1-((2-oxopyrrolidin-1-yl)methyl)-1,2,3,4-tetrahydroisoquinoline-2-carbonyl)-N-methylcyclohexane-1-carboxamide;
(S)-3-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-N-methylmorpholine-4-carboxamide;
(S)-2-((S)-5-chloro-8-((1-methyl-1H-1,2,3-triazol-4-yl)methoxy)-1-((2-oxopyrrolidin-1-yl)methyl)-1,2,3,4-tetrahydroisoquinoline-2-carbonyl)-N-methylpyrrolidine-1-carboxamide;
(1S,2R)-2-((S)-5-chloro-8-((1,5-dimethyl-1H-1,2,3-triazol-4-yl)methoxy)-1-((2-oxopyrrolidin-1-yl)methyl)-1,2,3,4-tetrahydroisoquinoline-2-carbonyl)-N,1-dimethylcyclohexane-1-carboxamide;
(1S,2R)-2-((S)-5-chloro-8-((1-methyl-5-(trifluoromethyl)-1H-1,2,3-triazol-4-yl)methoxy)-1-((2-oxopyrrolidin-1-yl)methyl)-1,2,3,4-tetrahydroisoquinoline-2-carbonyl)-N,1-dimethylcyclohexane-1-carboxamide;
(S)-2-((S)-5-chloro-8-((1-methyl-1H-1,2,3-triazol-4-yl)methoxy)-1-((2-oxopyrrolidin-1-yl)methyl)-1,2,3,4-tetrahydroisoquinoline-2-carbonyl)-N-methylpiperidine-1-carboxamide;
(1S,2R)-2-((S)-5-chloro-8-((1-methyl-1H-1,2,3-triazol-4-yl)methoxy)-1-((2-oxopyrrolidin-1-yl)methyl)-1,2,3,4-tetrahydroisoquinoline-2-carbonyl)-N,1-dimethylcyclohexane-1-carboxamide;
(1S,2R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-N,1-dimethylcyclopentane-1-carboxamide;
(1S,2R)-2-((1S)-5-chloro-8-((5-(difluoromethyl)-1-methyl-1H-1,2,3-triazol-4-yl)methoxy)-1-((3-methyl-2-oxopyrrolidin-1-yl)methyl)-1,2,3,4-tetrahydroisoquinoline-2-carbonyl)-N,1-dimethylcyclopentane-1-carboxamide;
(1S,2R)-2-((1S)-5-chloro-8-((5-(difluoromethyl)-1-methyl-1H-1,2,3-triazol-4-yl)methoxy)-1-((3-methyl-2-oxopyrrolidin-1-yl)methyl)-1,2,3,4-tetrahydroisoquinoline-2-carbonyl)-N,1-dimethylcyclohexane-1-carboxamide;
(1S,2R)-2-((S)-5-chloro-8-((1-methyl-5-(trifluoromethyl)-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-N,1-dimethylcyclohexane-1-carboxamide;
(1S,2R)-2-((S)-5-chloro-8-((5-cyclopropyl-1-methyl-1H-1,2,3-triazol-4-yl)methoxy)-1-((2-oxopyrrolidin-1-yl)methyl)-1,2,3,4-tetrahydroisoquinoline-2-carbonyl)-N,1-dimethylcyclohexane-1-carboxamide;
(1S,2R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxamide;
(1S,2R)-2-((S)-5-chloro-8-((1,5-dimethyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-N,1-dimethylcyclohexane-1-carboxamide;
(1S,2R)-2-((S)-5-chloro-8-((1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-N,1-dimethylcyclohexane-1-carboxamide;
(1S,2R)-2-((S)-5-chloro-8-((5-cyclopropyl-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-N,1-dimethylcyclohexane-1-carboxamide;
(1S,2R)-2-((S)-5-chloro-8-((1-methyl-1H-1,2,3-triazol-4-yl)methoxy)-1-(((R)-4-methyl-2-oxopyrrolidin-1-yl)methyl)-1,2,3,4-tetrahydroisoquinoline-2-carbonyl)-N,1-dimethylcyclohexane-1-carboxamide;
(1S,2R)-2-((S)-5-chloro-8-((5-(methoxymethyl)-1-methyl-1H-1,2,3-triazol-4-yl)methoxy)-1-(((R)-4-methyl-2-oxopyrrolidin-1-yl)methyl)-1,2,3,4-tetrahydroisoquinoline-2-carbonyl)-N,1-dimethylcyclohexane-1-carboxamide;
(1S,2R)-2-((S)-5-chloro-8-((5-(difluoromethyl)-1-methyl-1H-1,2,3-triazol-4-yl)methoxy)-7-fluoro-1-((6-oxo-5-azaspiro[2.4]heptan-5-yl)methyl)-1,2,3,4-tetrahydroisoquinoline-2-carbonyl)-N,1-dimethylcyclohexane-1-carboxamide;
(1S,2R)-2-((S)-5-chloro-8-((5-(difluoromethyl)-1-methyl-1H-1,2,3-triazol-4-yl)methoxy)-7-fluoro-1-((6-oxo-5-azaspiro[2.4]heptan-5-yl)methyl)-1,2,3,4-tetrahydroisoquinoline-2-carbonyl)-1-methylcyclohexane-1-carboxamide;
(1S,2R)-2-((S)-5-chloro-8-((5-(difluoromethyl)-1-methyl-1H-1,2,3-triazol-4-yl)methoxy)-7-fluoro-1-((3-oxomorpholino)methyl)-1,2,3,4-tetrahydroisoquinoline-2-carbonyl)-N,1-dimethylcyclohexane-1-carboxamide;
(1S,2R)-2-((S)-5-chloro-8-((5-(difluoromethyl)-1-methyl-1H-1,2,3-triazol-4-yl)methoxy)-7-fluoro-1-((2-oxopyrrolidin-1-yl)methyl)-1,2,3,4-tetrahydroisoquinoline-2-carbonyl)-N,1-dimethylcyclohexane-1-carboxamide;
(1S,2R)-2-((S)-5-chloro-7-fluoro-8-((1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-N,1-dimethylcyclohexane-1-carboxamide;
(1S,2R)-2-((S)-5-chloro-7-fluoro-1-((6-oxo-5-azaspiro[2.4]heptan-5-yl)methyl)-8-((4,5,6,7-tetrahydro-[1,2,3]triazolo[1,5-a]pyridin-3-yl)methoxy)-1,2,3,4-tetrahydroisoquinoline-2-carbonyl)-1-methylcyclohexane-1-carboxamide;
(1S,2R)-2-((S)-5-chloro-8-((1,5-dimethyl-1H-1,2,3-triazol-4-yl)methoxy)-7-fluoro-1-((6-oxo-5-azaspiro[2.4]heptan-5-yl)methyl)-1,2,3,4-tetrahydroisoquinoline-2-carbonyl)-1-methylcyclohexane-1-carboxamide;
(1S,2R)-2-((S)-5-chloro-8-((5-cyclopropyl-1-methyl-1H-1,2,3-triazol-4-yl)methoxy)-1-(((R)-4-methyl-2-oxopyrrolidin-1-yl)methyl)-1,2,3,4-tetrahydroisoquinoline-2-carbonyl)-N,1-dimethylcyclohexane-1-carboxamide; and
(1S,2R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-N-methylcyclohexane-1-carboxamide.

In an embodiment, the compound of Formula I is selected from any one of the following compounds, or a pharmaceutically acceptable salt thereof:

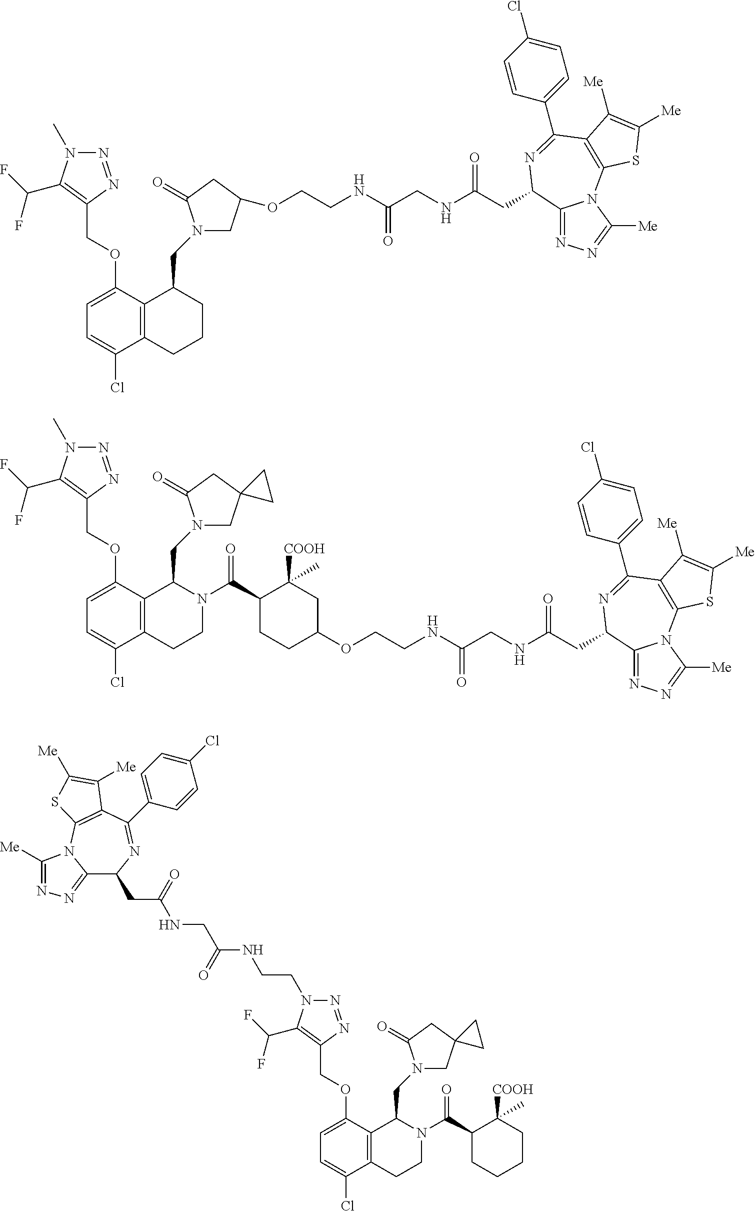 [SMILES: Cc1sc2c(c1C)C(c1ccc(Cl)cc1)=N[C@@H](CC(=O)NCC(=O)NCCOC1CC(=O)N(C[C@H]3CCCc4c(Cl)ccc(OCc5nnn(C)c5C(F)F)c43)C1)c1nnc(C)n1-2 ; Cc1sc2c(c1C)C(c1ccc(Cl)cc1)=N[C@@H](CC(=O)NCC(=O)NCCOC1CC[C@@H](C(=O)N3CCc4c(Cl)ccc(OCc5nnn(C)c5C(F)F)c4[C@H]3CN3CC4(CC4)CC3=O)[C@@](C)(C(=O)O)C1)c1nnc(C)n1-2 ; Cc1sc2c(c1C)C(c1ccc(Cl)cc1)=N[C@@H](CC(=O)NCC(=O)NCCn1nnc(COc3ccc(Cl)c4c3[C@@H](CN3CC5(CC5)CC3=O)N(C(=O)[C@@H]3CCCC[C@]3(C)C(=O)O)CC4)c1C(F)F)c1nnc(C)n1-2]

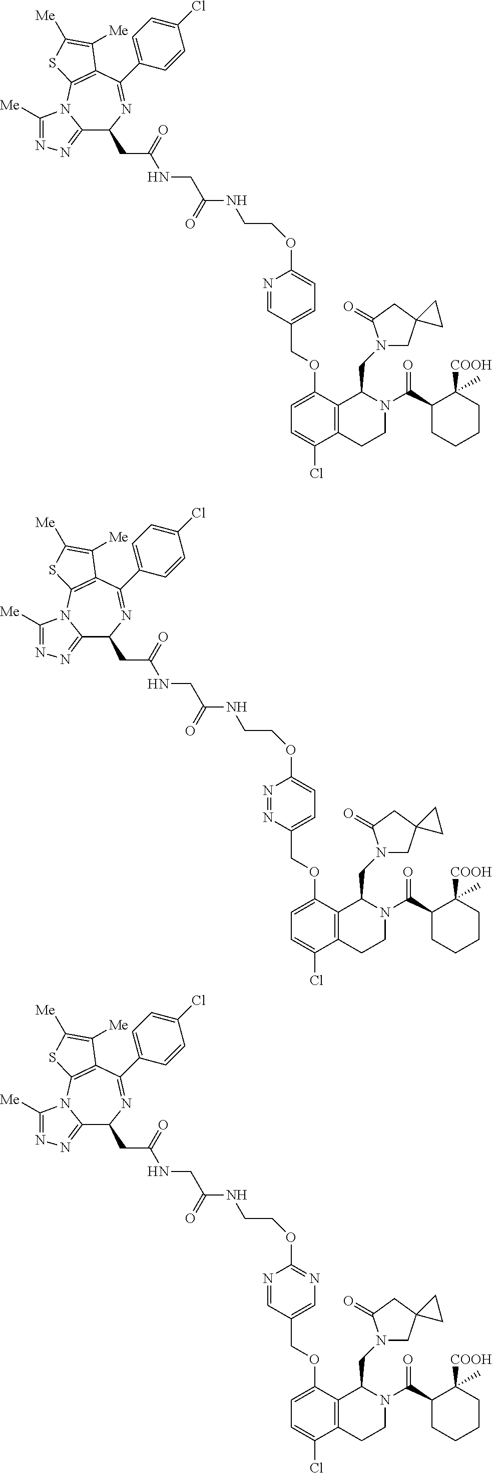 [SMILES: Cc1sc2c(c1C)C(c1ccc(Cl)cc1)=N[C@@H](CC(=O)NCC(=O)NCCOc1ccc(COc3ccc(Cl)c4c3[C@@H](CN3CC5(CC5)CC3=O)N(C(=O)[C@@H]3CCCC[C@]3(C)C(=O)O)CC4)cn1)c1nnc(C)n1-2 ; Cc1sc2c(c1C)C(c1ccc(Cl)cc1)=N[C@@H](CC(=O)NCC(=O)NCCOc1ccc(COc3ccc(Cl)c4c3[C@@H](CN3CC5(CC5)CC3=O)N(C(=O)[C@@H]3CCCC[C@]3(C)C(=O)O)CC4)nn1)c1nnc(C)n1-2 ; Cc1sc2c(c1C)C(c1ccc(Cl)cc1)=N[C@@H](CC(=O)NCC(=O)NCCOc1ncc(COc3ccc(Cl)c4c3[C@@H](CN3CC5(CC5)CC3=O)N(C(=O)[C@@H]3CCCC[C@]3(C)C(=O)O)CC4)cn1)c1nnc(C)n1-2]

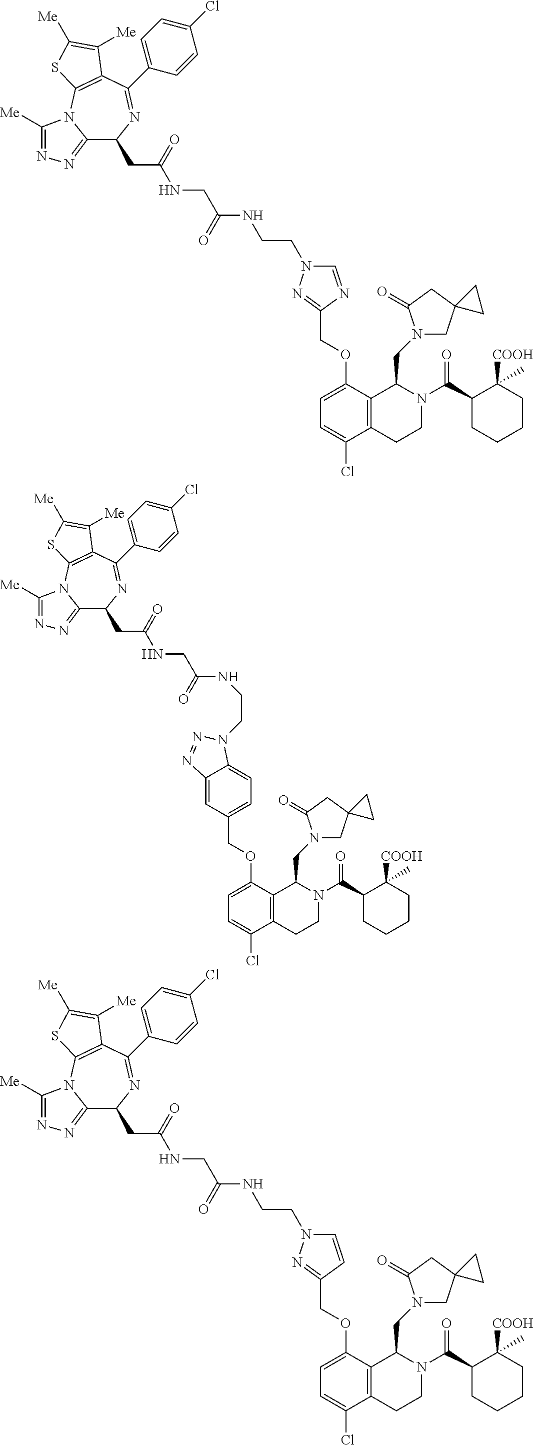 [SMILES: Cc1sc2c(c1C)C(c1ccc(Cl)cc1)=N[C@@H](CC(=O)NCC(=O)NCCn1cnc(COc3ccc(Cl)c4c3[C@@H](CN3CC5(CC5)CC3=O)N(C(=O)[C@@H]3CCCC[C@]3(C)C(=O)O)CC4)n1)c1nnc(C)n1-2 ; Cc1sc2c(c1C)C(c1ccc(Cl)cc1)=N[C@@H](CC(=O)NCC(=O)NCCn1nnc3cc(COc4ccc(Cl)c5c4[C@@H](CN4CC6(CC6)CC4=O)N(C(=O)[C@@H]4CCCC[C@]4(C)C(=O)O)CC5)ccc31)c1nnc(C)n1-2 ; Cc1sc2c(c1C)C(c1ccc(Cl)cc1)=N[C@@H](CC(=O)NCC(=O)NCCn1ccc(COc3ccc(Cl)c4c3[C@@H](CN3CC5(CC5)CC3=O)N(C(=O)[C@@H]3CCCC[C@]3(C)C(=O)O)CC4)n1)c1nnc(C)n1-2]

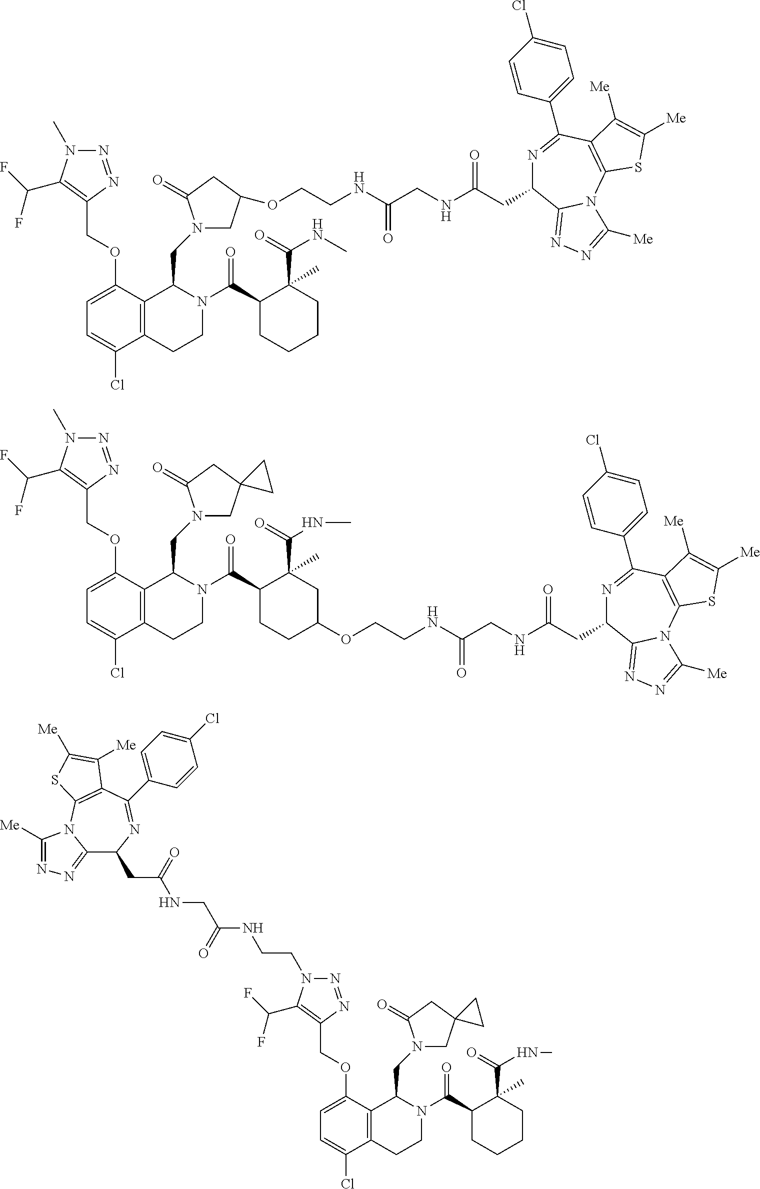 [SMILES: CNC(=O)[C@@]1(C)CCCC[C@H]1C(=O)N1CCc2c(Cl)ccc(OCc3nnn(C)c3C(F)F)c2[C@H]1CN1CC(OCCNC(=O)CNC(=O)C[C@@H]2N=C(c3ccc(Cl)cc3)c3c(sc(C)c3C)-n3c(C)nnc32)CC1=O ; CNC(=O)[C@@]1(C)CC(OCCNC(=O)CNC(=O)C[C@@H]2N=C(c3ccc(Cl)cc3)c3c(sc(C)c3C)-n3c(C)nnc32)CC[C@H]1C(=O)N1CCc2c(Cl)ccc(OCc3nnn(C)c3C(F)F)c2[C@H]1CN1CC2(CC2)CC1=O ; CNC(=O)[C@@]1(C)CCCC[C@H]1C(=O)N1CCc2c(Cl)ccc(OCc3nnn(CCNC(=O)CNC(=O)C[C@@H]4N=C(c5ccc(Cl)cc5)c5c(sc(C)c5C)-n5c(C)nnc54)c3C(F)F)c2[C@H]1CN1CC2(CC2)CC1=O]

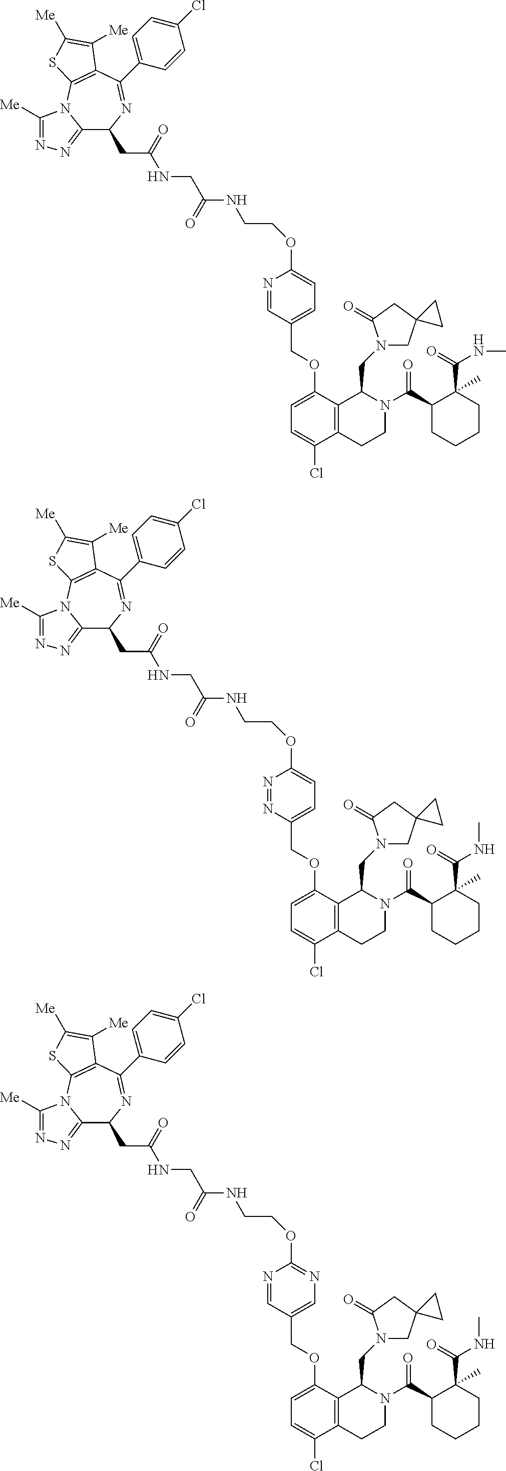 [SMILES: CNC(=O)[C@@]1(C)CCCC[C@H]1C(=O)N1CCc2c(Cl)ccc(OCc3ccc(OCCNC(=O)CNC(=O)C[C@@H]4N=C(c5ccc(Cl)cc5)c5c(sc(C)c5C)-n5c(C)nnc54)nc3)c2[C@H]1CN1CC2(CC2)CC1=O ; CNC(=O)[C@@]1(C)CCCC[C@H]1C(=O)N1CCc2c(Cl)ccc(OCc3ccc(OCCNC(=O)CNC(=O)C[C@@H]4N=C(c5ccc(Cl)cc5)c5c(sc(C)c5C)-n5c(C)nnc54)nn3)c2[C@H]1CN1CC2(CC2)CC1=O ; CNC(=O)[C@@]1(C)CCCC[C@H]1C(=O)N1CCc2c(Cl)ccc(OCc3cnc(OCCNC(=O)CNC(=O)C[C@@H]4N=C(c5ccc(Cl)cc5)c5c(sc(C)c5C)-n5c(C)nnc54)nc3)c2[C@H]1CN1CC2(CC2)CC1=O]

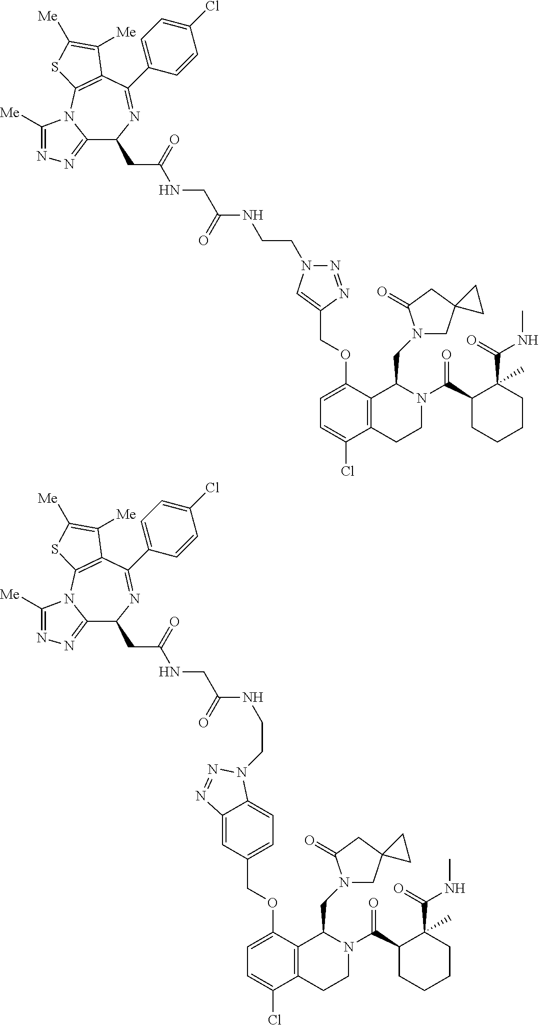 [SMILES: CNC(=O)[C@@]1(C)CCCC[C@H]1C(=O)N1CCc2c(Cl)ccc(OCc3cn(CCNC(=O)CNC(=O)C[C@@H]4N=C(c5ccc(Cl)cc5)c5c(sc(C)c5C)-n5c(C)nnc54)nn3)c2[C@H]1CN1CC2(CC2)CC1=O ; CNC(=O)[C@@]1(C)CCCC[C@H]1C(=O)N1CCc2c(Cl)ccc(OCc3ccc4c(c3)nnn4CCNC(=O)CNC(=O)C[C@@H]3N=C(c4ccc(Cl)cc4)c4c(sc(C)c4C)-n4c(C)nnc43)c2[C@H]1CN1CC2(CC2)CC1=O]

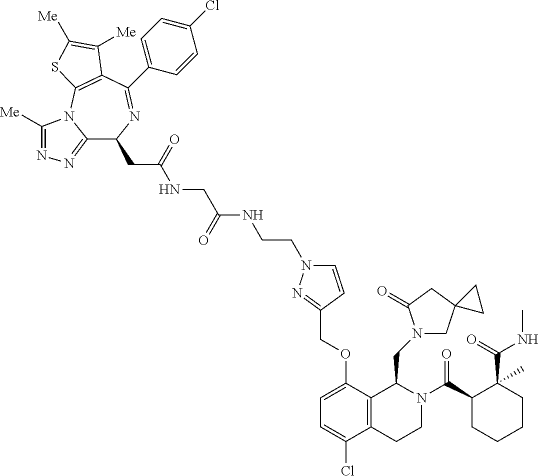 [SMILES: CNC(=O)[C@@]1(C)CCCC[C@H]1C(=O)N1CCc2c(Cl)ccc(OCc3ccn(CCNC(=O)CNC(=O)C[C@@H]4N=C(c5ccc(Cl)cc5)c5c(sc(C)c5C)-n5c(C)nnc54)n3)c2[C@H]1CN1CC2(CC2)CC1=O]

In an embodiment, the compound of Formula I is selected from any one of the following compounds, or a pharmaceutically acceptable salt thereof:

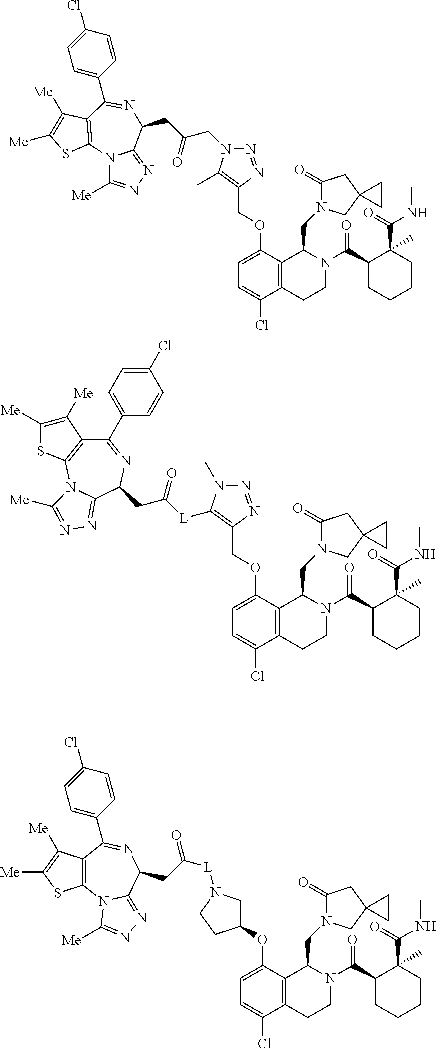

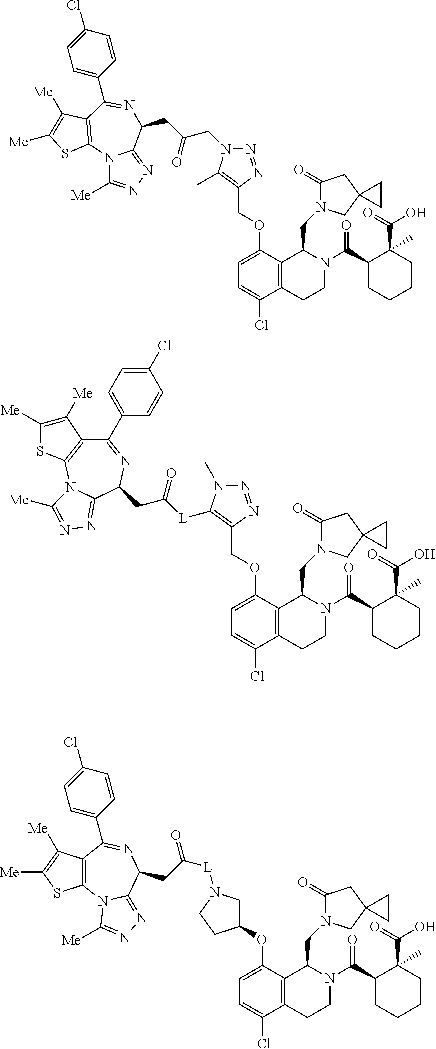

wherein L is independently selected from one of the following bivalent linker groups:

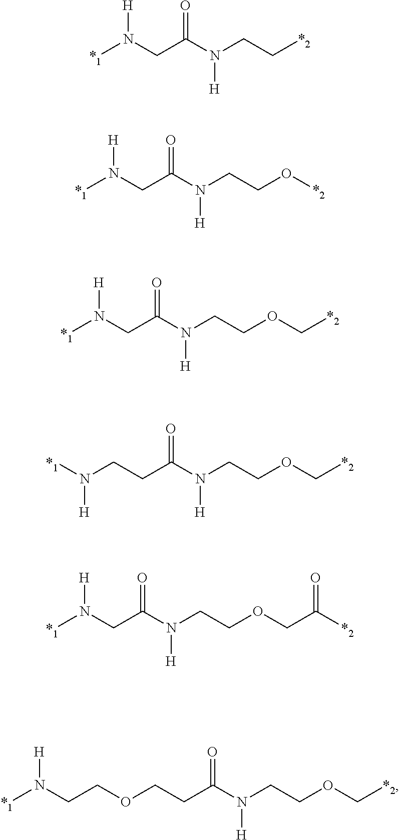

wherein *1 is the point of attachment to the PBG and *2 is the point of attachment to the KBG.

In an embodiment, the compound of Formula I is selected from any one of Example compounds 1 to 6, described hereinbelow, or a pharmaceutically acceptable salt thereof.

Suitable or preferred features of any compounds of the present invention may also be suitable features of any other aspect.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterized by the absolute configuration of its asymmetric centre and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention typically possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers, diastereoisomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess Nrf2 activation activity.

The present invention also encompasses compounds of the invention as defined herein which comprise one or more isotopic substitutions. For example, H may be in any isotopic form, including ¹H, ²H (D) and ³H (T); C may be in any isotopic form including ¹²C, ¹³C, and ¹⁴C; and O may be in any isotopic form, including ¹⁶O and ¹⁸O; and the like.

It is also to be understood that certain compounds of the invention may exist in solvated as well as unsolvated forms such as, for example, hydrated forms. It is to be understood that the invention encompasses all such solvated forms that possess Nrf2 activation activity.

It is also to be understood that certain compounds of the invention may exhibit polymorphism, and that the invention encompasses all such forms that possess Nrf2 activation activity.

Compounds of the invention may exist in a number of different tautomeric forms and references to compounds of the invention include all such forms. For the avoidance of doubt, where a compound can exist in one of several tautomeric forms, and only one is specifically described or shown, all others are nevertheless embraced by compounds of the invention. Examples of tautomeric forms include keto-, enol-, and enolate-forms, as in, for example, the following tautomeric pairs: keto/enol, imine/enamine, amide/imino alcohol, amidine/amidine, nitroso/oxime, thioketone/enethiol, and nitro/aci-nitro.

Compounds of the invention containing an amine function may also form N-oxides. A reference herein to a compound of the formula I that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

The compounds of the invention may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the invention and in-vivo cleavable amide derivatives that may be formed at a carboxy group or an amino group in a compound of the invention.

Accordingly, the present invention includes those compounds of the formula I as defined hereinbefore when made available by organic synthesis and when made available within the human or animal body by way of cleavage of a pro-drug thereof. Accordingly, the present invention includes those compounds of the formula I that are produced by organic synthetic means and also such compounds that are produced in the human or animal body by way of metabolism of a precursor compound, that is a compound of the formula I may be a synthetically-produced compound or a metabolically-produced compound.

A suitable pharmaceutically acceptable pro-drug of a compound of the formula I is one that is based on reasonable medical judgement as being suitable for administration to the human or animal body without undesirable pharmacological activities and without undue toxicity.

Various forms of pro-drug have been described, for example in the following documents:
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the formula I that possesses a carboxy group is, for example, an in vivo cleavable ester thereof. An in vivo cleavable ester of a compound of the formula I containing a carboxy group is, for example, a pharmaceutically acceptable ester which is cleaved in the human or animal body to produce the parent acid. Suitable pharmaceutically acceptable esters for carboxy include C₁₋₆alkyl esters such as methyl, ethyl and tert-butyl, C₁₋₆alkoxymethyl esters such as methoxymethyl esters, C₁₋₆alkanoyloxymethyl esters such as pivaloyloxymethyl esters, 3-phthalidyl esters, C₃₋₈cycloalkylcarbonyloxy-C₁₋₆alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C₁₋₆alkoxycarbonyloxy-C₁₋₆alkyl esters such as methoxycarbonyloxymethyl and 1-methoxycarbonyloxyethyl esters.

A suitable pharmaceutically acceptable pro-drug of a compound of the formula I that possesses a hydroxy group is, for example, an in vivo cleavable ester or ether thereof. An in vivo cleavable ester or ether of a compound of the formula I containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁₋₁₀alkanoyl groups such as acetyl, benzoyl, phenylacetyl and substituted benzoyl and phenylacetyl groups, C₁₋₁₀alkoxycarbonyl groups such as ethoxycarbonyl, N,N—(C₁₋₆)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl. Suitable pharmaceutically acceptable ether forming groups for a hydroxy group include a-acyloxyalkyl groups such as acetoxymethyl and pivaloyloxymethyl groups.

A suitable pharmaceutically acceptable pro-drug of a compound of the formula I that possesses a carboxy group is, for example, an in vivo cleavable amide thereof, for example an amide formed with an amine such as ammonia, a C₁₋₄alkylamine such as methylamine, a (C₁₋₄alkyl)₂ amine such as dimethylamine, N-ethyl-N-methylamine or diethylamine, a C₁₋₄alkoxy-C₂₋₄alkylamine such as 2-methoxyethylamine, a phenyl-C₁₋₄alkylamine such as benzylamine and amino acids such as glycine or an ester thereof.

A suitable pharmaceutically acceptable pro-drug of a compound of the formula I that possesses an amino group is, for example, an in vivo cleavable amide derivative thereof. Suitable pharmaceutically acceptable amides from an amino group include, for example an amide formed with C₁₋₁₀alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl.

The in vivo effects of a compound of the formula I may be exerted in part by one or more metabolites that are formed within the human or animal body after administration of a compound of the formula 1. As stated hereinbefore, the in vivo effects of a compound of the formula I may also be exerted by way of metabolism of a precursor compound (a pro-drug).

It shall also be appreciated that compounds of the formula I may also be covalently linked (at any suitable position) to other groups such as, for example, solubilising moieties (for example, PEG polymers), moieties that enable them to be bound to a solid support (such as, for example, biotin-containing moieties), and targeting ligands (such as antibodies or antibody fragments).

### Synthesis

In the description of the synthetic methods described below and in the referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

Necessary starting materials may be obtained by standard procedures of organic chemistry. The preparation of such starting materials is described in conjunction with the following representative process variants and within the accompanying Examples. Alternatively, necessary starting materials are obtainable by analogous procedures to those illustrated which are within the ordinary skill of an organic chemist.

It will be appreciated that during the synthesis of the compounds of the invention in the processes defined below, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, “Protecting groups in Organic Synthesis (3^{rd} Ed), John Wiley & Sons, NY (1999)”, T. Greene & P. Wuts. Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively, an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example BF₃·OEt₂. A suitable alternative protecting group for a primary amino group is, for example, a phthaloyl group which may be removed by treatment with an alkylamine, for example dimethylaminopropylamine, or with hydrazine.

The person skilled in the art will recognise that the compounds of the invention may be prepared, in known manner, in a variety of ways. Compounds of formula I can be prepared by the methods given below, by the methods given in the experimental or by analogous methods. The routes described are merely illustrative of some of the methods that can be employed for the synthesis of compounds of formula I and the person skilled in the art will appreciate that the order of the reaction steps is not limited to those described. It will also be appreciated that the assignment of nucleophile and electrophile is not limited to that described herein and in some cases it may be appropriate for the assignment to be reversed. Different approaches to synthetic chemistry strategy are described in “Organic Synthesis: The Disconnection Approach”, 2^{nd} edition, S. Warren and P. Wyatt (2008).

The synthesis of example KEAP1 binding groups (KBGs) of the invention are described in WO 2020/084300 and WO 2021/214472.

The synthesis of the BRD4 ligand attached to a range of linkers is described in doi.org/10.1038/s41598-020-72491-9 and doi.org/10.1021/jacs.1c04841.

### General Method A

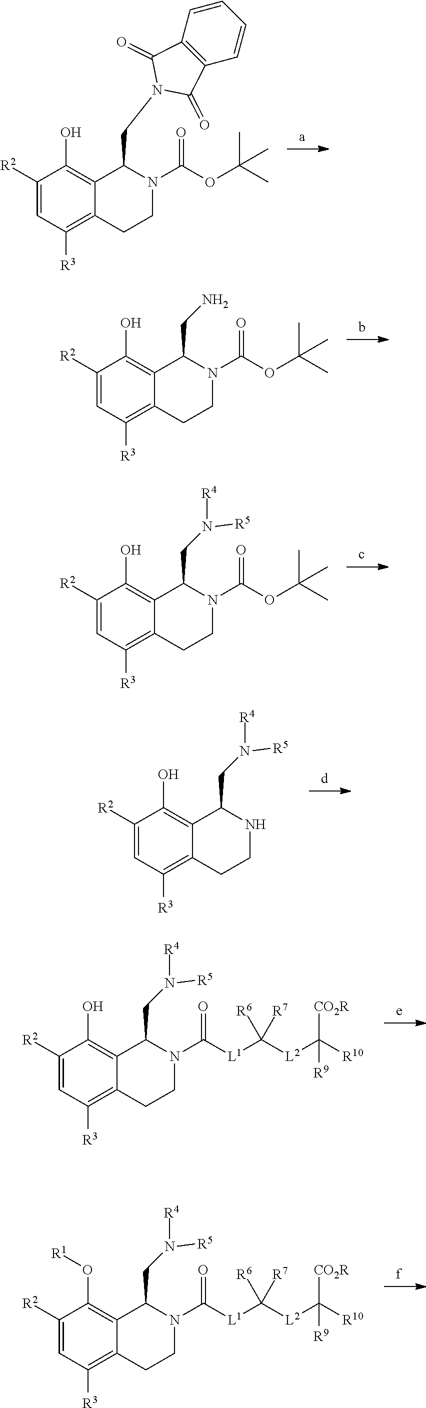

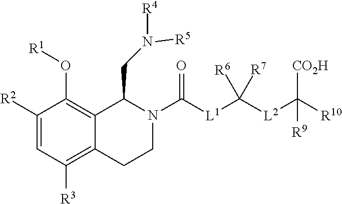

In a typical synthetic procedure the phthalimide, when used as a protecting group, is removed using typical reagents (e.g. hydrazine) and the amine is reacted with appropriate reagents to install the desired substitution NR⁴R5. In a third step the Boc protecting group (CO₂Bu) is then removed typically by treatment with HCl. The L¹C(R⁶)(R⁷)L²C(R⁸)(R⁹)R¹⁰ group may be introduced in a fourth step from the appropriately substituted and protected bis-acid derivative; ideally where one of the acid groups is activated for reaction with the amine of the tetrahydroisoquinoline (THIQ) scaffold and the other acid group is suitably protected, for example as a benzyl or dimethoxybenzyl ester, or by ring opening of an appropriate cyclic anhydride, or by reaction with an acid chloride. Typical amide coupling reagents such as HATU are used to effect acid activation. A fifth step involves installation of the required ether through conventional methods such as alkylation with an alkyl halide or activated alcohol (e.g. mesylate, triflate) or Mitsunobu reaction using reagents such as DBAD or DEAD and an appropriate phosphine. The required ether may be the linker-POI or an appropriate ether with the required functionality or protecting group to install the linker-POI in additional steps. In a final step, the protecting group is removed from the carboxylic acid by the appropriate methodology such as hydrolysis, hydrogenolysis, strong acid such as HCl or TFA or Lewis acid such as BBr₃.

### General Method B

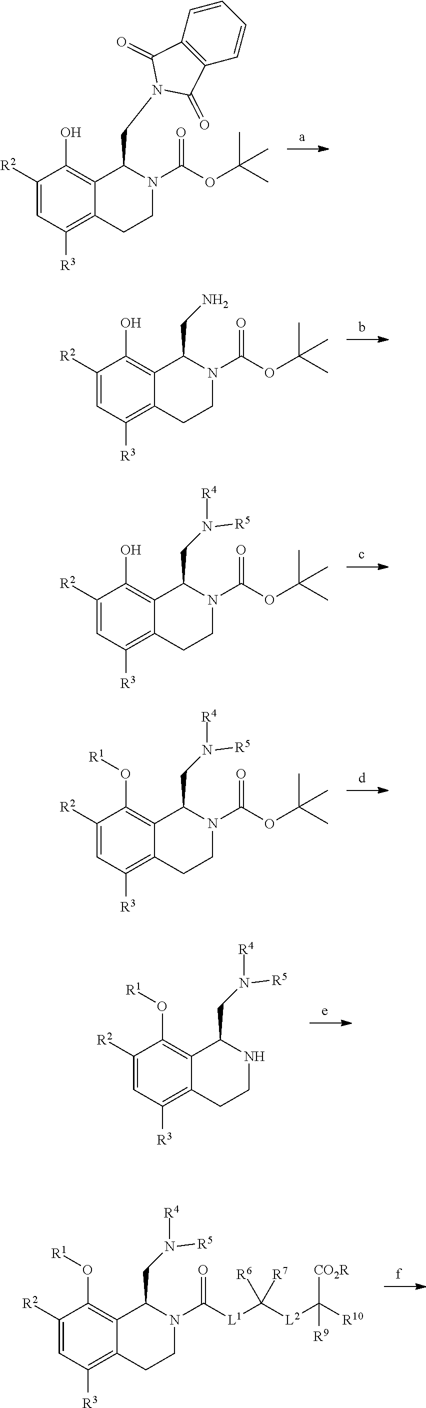

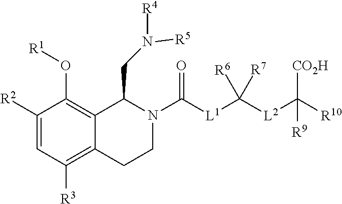

In a further typical procedure, the order of steps can be altered compared with general method A. The first two steps are carried out as described in general method A. A third step involves installation of the required ether through conventional methods such as alkylation with an alkyl halide or activated alcohol (e.g. mesylate, triflate) or Mitsunobu reaction using reagents such as DBAD or DEAD and an appropriate phosphine. The required ether may be the linker-POI or an appropriate ether with the required functionality or protecting group to install the linker-POI in a later step. The Boc protecting group is then removed typically by treatment with HCl. The L¹C(R⁶)(R⁷)L²C(R³)(R⁹)R¹⁰ group may be introduced from the appropriately substituted and protected bis-acid derivative, as described in General Method A. In a final step, the protecting group is removed from the carboxylic acid by the appropriate methodology such as hydrolysis, hydrogenolysis, strong acid such as HCl or TFA or Lewis acid such as BBr₃.

### General Method C

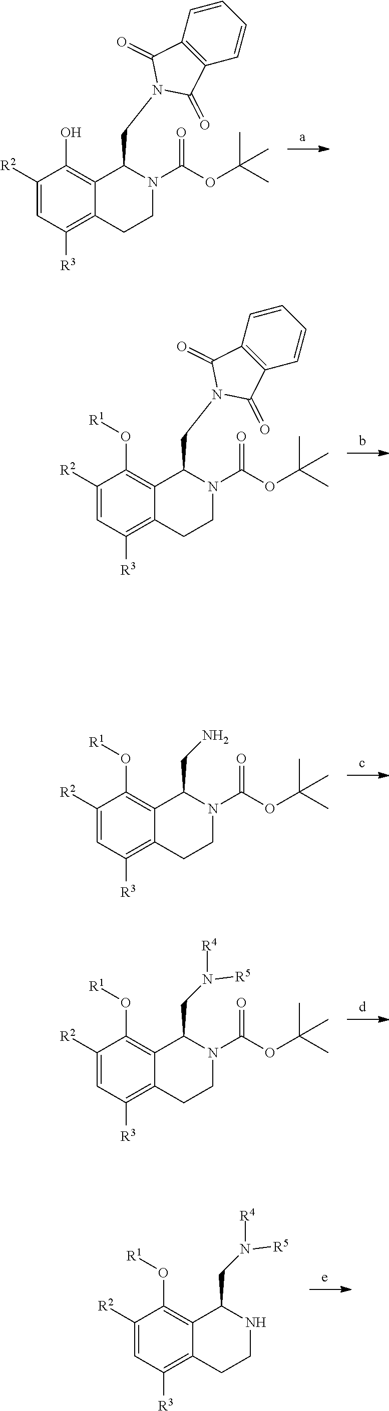

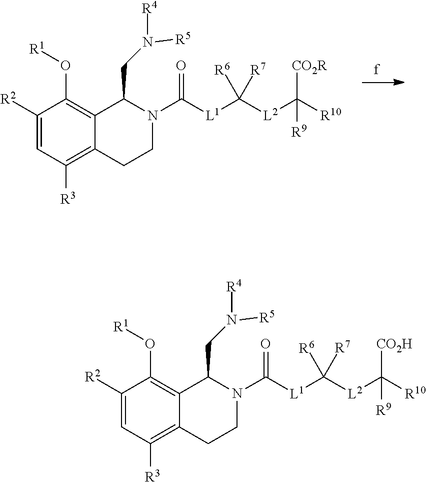

In a further typical procedure, the order of steps can be altered compared with general methods A and B. The required ether is installed in a first step, again through conventional methods as described in the above General Methods A and B. The phthalimide protecting group is removed in a second step and the amine is reacted with appropriate reagents to install the desired substitution NR⁴R⁵. The Boc protecting group is then removed and the L¹C(R⁶)(R⁷)L²C(R³)(R⁹)R¹⁰ group may then be introduced in a fifth step. The final step comprises removal of the carboxylic acid protecting group as described in the above general methods.

### General Method D

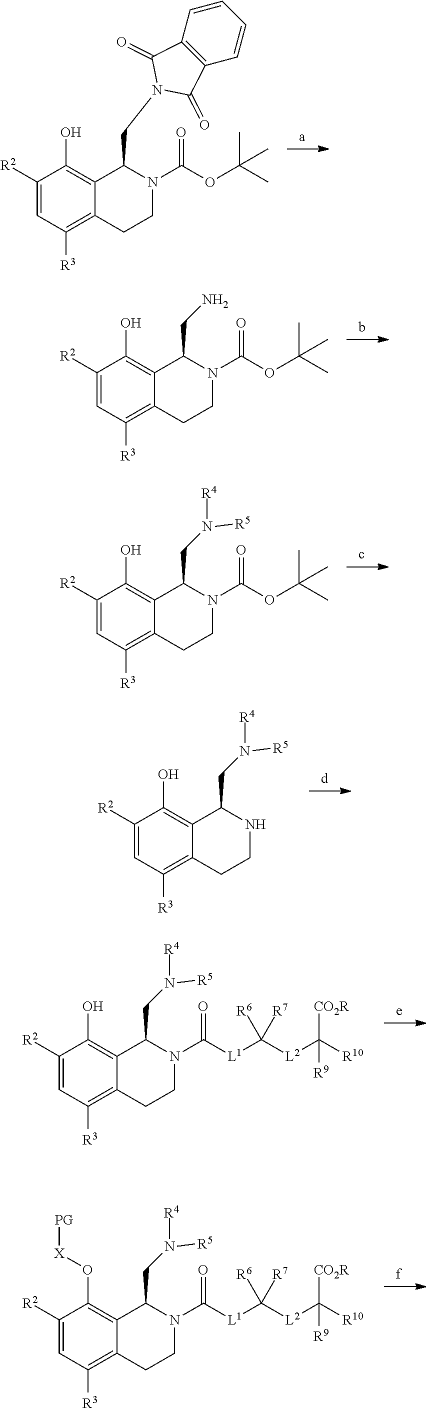

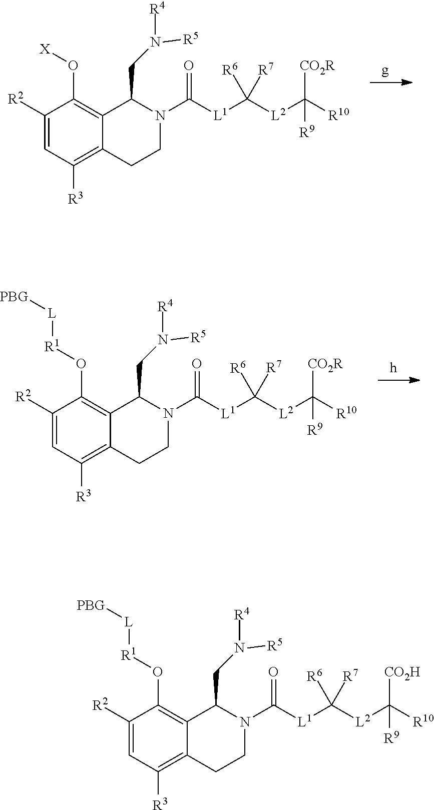

In a further typical procedure, General Method A can be modified to incorporate a functional group bearing a protecting group that may be reacted to form a covalent bond within the linker. Steps 1-4 are carried out as described in General Method A. In a fifth step a group X-PG is introduced wherein PG is a protecting group, for example a heterocycle bearing a BOC or phthalimide protecting group. In a sixth step this protecting group (PG) may be deprotected using appropriate methods and in a seventh step the reactive functionality, for instance an amine or alcohol, may be further reacted to install the required linker and PBG. The final step comprises removal of the carboxylic acid protecting group as described in the above general methods.

The THIQ scaffold wherein R³ is chloro may be constructed according to the route outlined below:

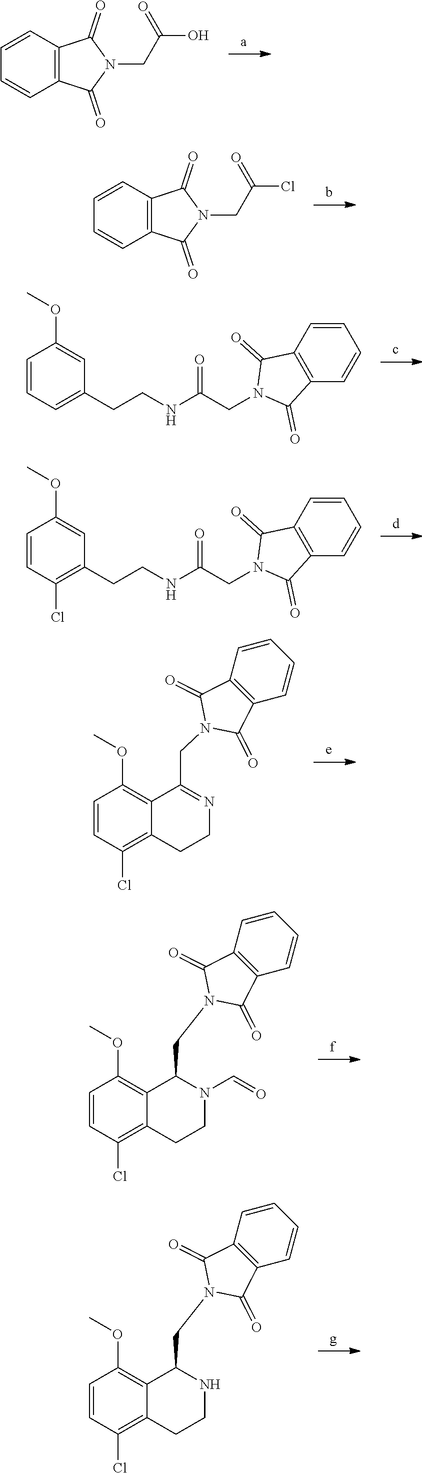

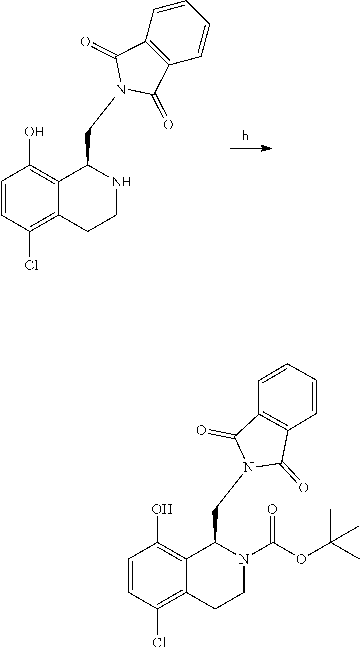 [SMILES: O=C1c2ccccc2C(=O)N1C[C@H]1NCCc2c(Cl)ccc(O)c21 ; CC(C)(C)OC(=O)N1CCc2c(Cl)ccc(O)c2[C@H]1CN1C(=O)c2ccccc2C1=O]

Scheme 1: a) SOCl₂, EtOAc; b) 2-(3-methoxyphenyl)ethan-1-amine, Et₃N, DCM; c) NCS, DMF; d) P₂O₅, MeCN; e) benzeneruthenium(II) chloride dimer, (1S,2S)-(+)—N-p-tosyl-1,2-diphenylethylene-diamine, Et₃N, HCO₂H, MeCN; f) HCl, THF; g) BBr₃, DCM; h) Boc₂O, DCM.

In the above general methods, KBGs having an amide group (R⁸=C(O)NR^{8a}R^{8b}) may be prepared from the corresponding acid group (R⁸=CO₂H) using amide coupling reactions and agents well known in the art, such as HATU and 1,1′-carbonyldiimidazole (CDI).

### General Method for PBG-L-KBG (R¹ Attachment)

Compounds of the invention where the PBG-linker(L) is attached to the R¹ group of a KBG may be prepared from suitable intermediates prepared using the scheme below, where X is any suitable linker chain as defined herein:

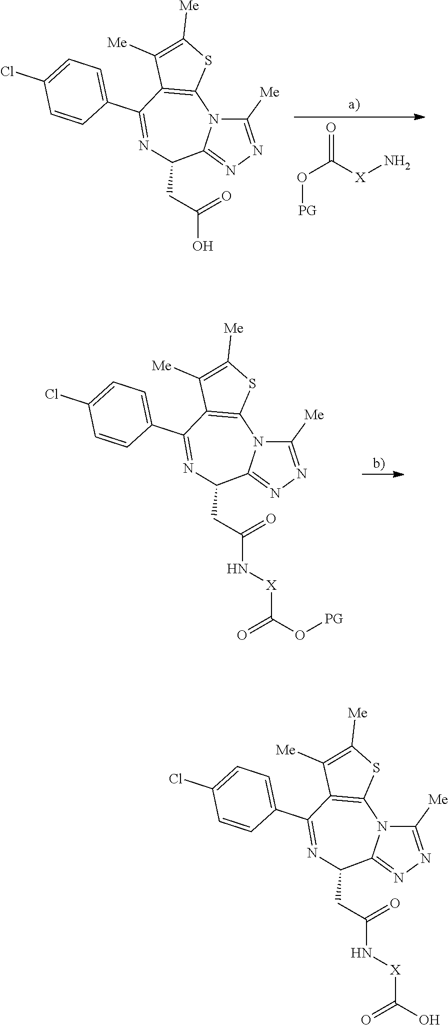

Scheme 2: a) EDCl, HOAt, NMM, b) TFA

Coupling of the PBG ligand (here exemplified with the JQ-1 ligand—a BRD4 binder) with an appropriately protected amino acid linker followed by suitable deprotection provides a PBG-Linker-COOH compound that can be coupled to the R¹ group through subsequent amide formation.

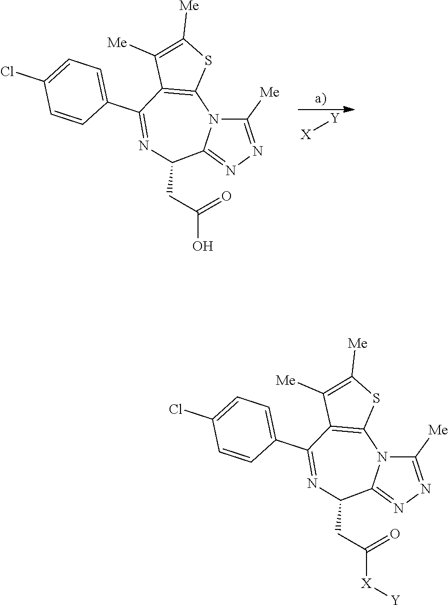

Scheme 3: a) HATU, DIPEA, DMF

Alternatively, coupling of the PBG ligand (here exemplified with the JQ-1 ligand—a BRD4 binder) with an X—Y compound (where X is any suitable linker chain as defined herein and Y is an appropriate functionality, such as an alcohol) provides a PBG-Linker-Y compound that can be coupled directly to the KBG R¹ group through appropriate methods. X may be an amine to provide an amide-linked compound or an alcohol to provide an ester-linked compound. Example transformations to attach these compounds to the KBG may include, but are not limited to, ether formation using an alkyl halide or activated alcohol (e.g. mesylate, triflate) or Mitsunobu reaction using reagents such as DBAD or DEAD and an appropriate phosphine, or ester formation using an appropriate alcohol.

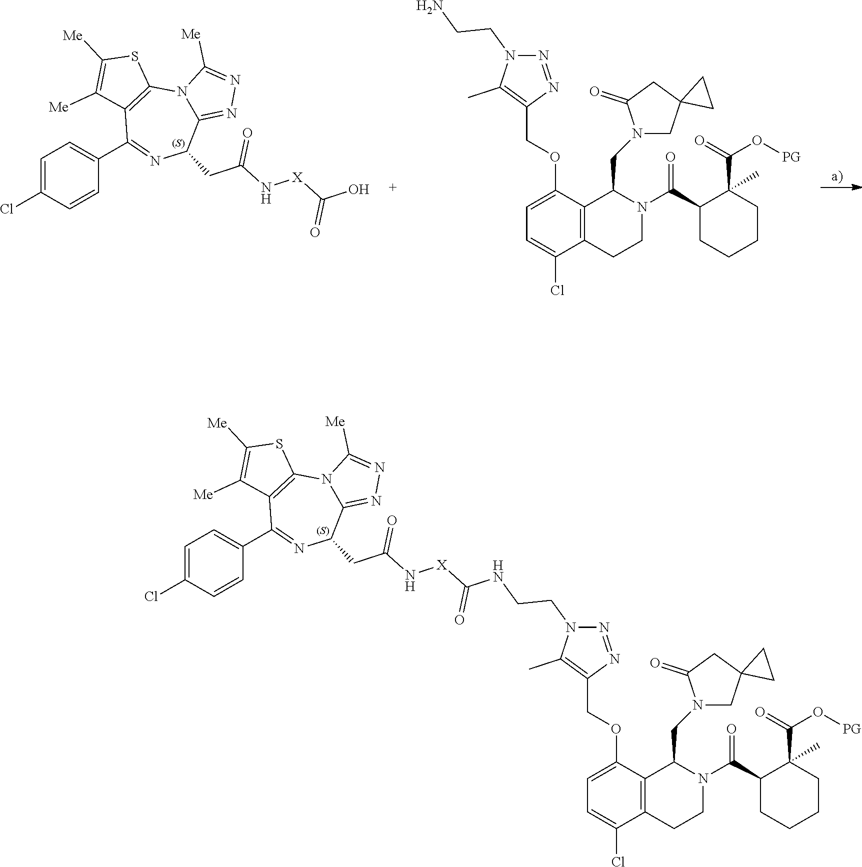

Scheme 4: a) HATU, DIPEA, DMF

In Scheme 4 the functionalised PBG-Linker compound (here exemplified as a carboxylic acid) can be coupled to the R¹ group (here exemplified using a substituted aminoethyl triazole) through subsequent amide formation using typical amide coupling conditions, for example using amide coupling reagents such as HATU.

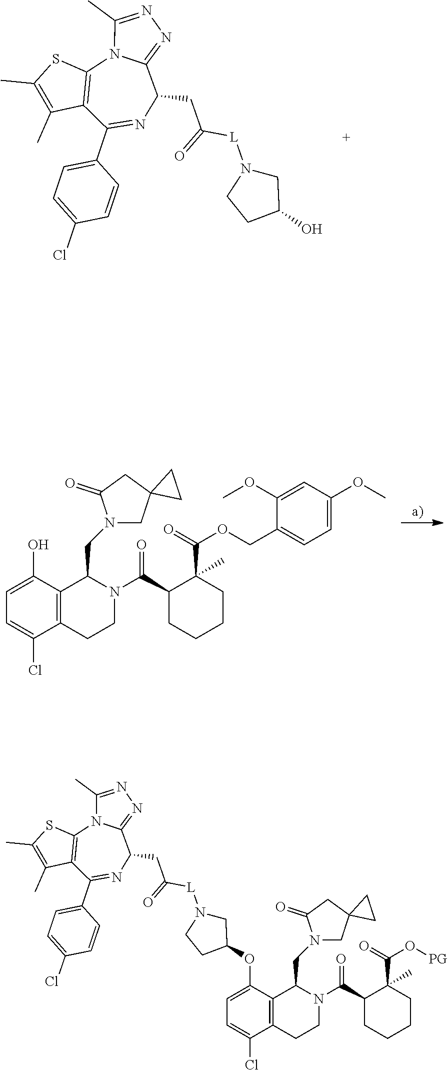

Scheme 5: a) PPh₃, DEAD, THE

Alternatively, the PBG-Linker-Y compound (here exemplified with the JQ-1 ligand—a BRD4 binder, with a pendant alcohol group) can be coupled directly to the KBG R¹ group through appropriate methods, such as ether formation using an alkyl halide or activated alcohol (e.g. mesylate, triflate) or Mitsunobu reaction (as exemplified in Scheme 5) using reagents such as DBAD or DEAD and an appropriate phosphine, or ester formation using an alcohol.

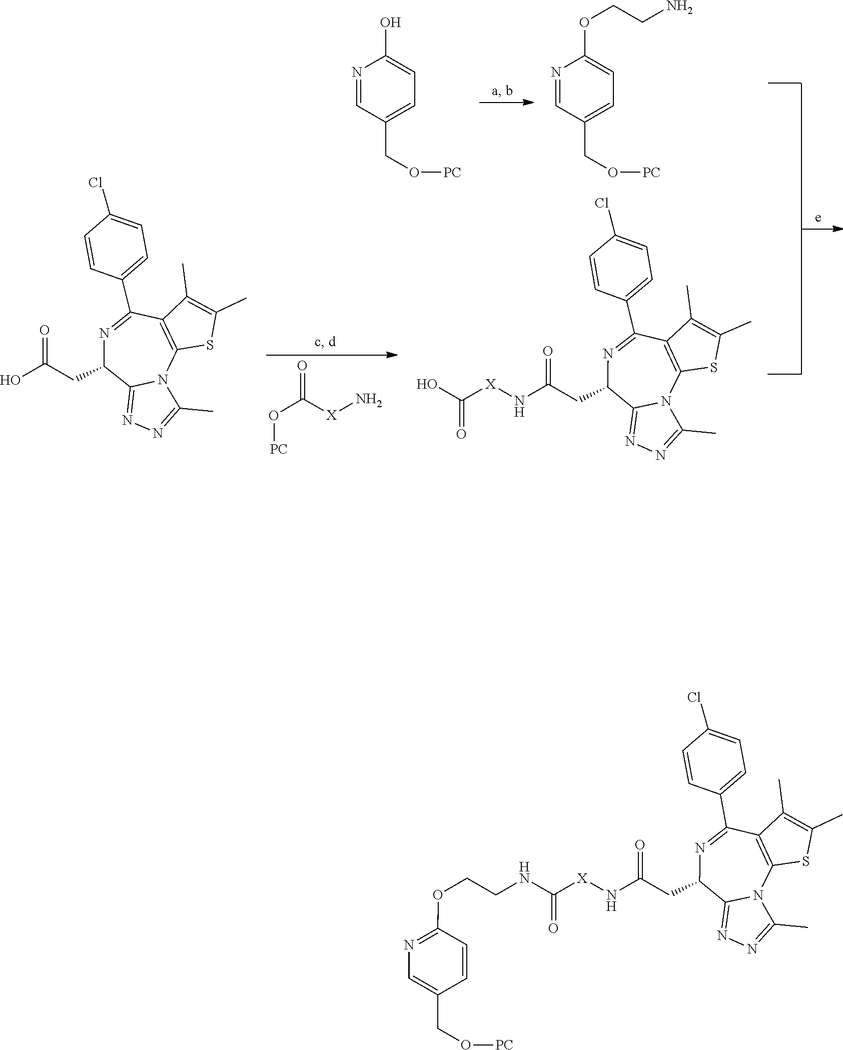

Scheme 6: a) Cs₂CO₃, DMF, tert-butyl (2-bromoethyl) carbamate; b) TFA/DCM; c) HOAt, EDCl; d) acid deprotection; e) HOAt, EDCl.

The PBG-Linker-COOH compound may contain suitable heterocycles (such as the pyridine ring as exemplified above in Scheme 6) that can be coupled to the R¹ group through subsequent amide formation or other well-known methods may be employed to attach an aminoethyl group.

General Method for PBG-L-KBG (—NR⁴R⁵ Attachment)

Compounds of the invention where the PBG-linker(L) is attached to the —NR⁴R⁵ group of a KBG may be prepared from suitable intermediates as shown in Scheme 7:

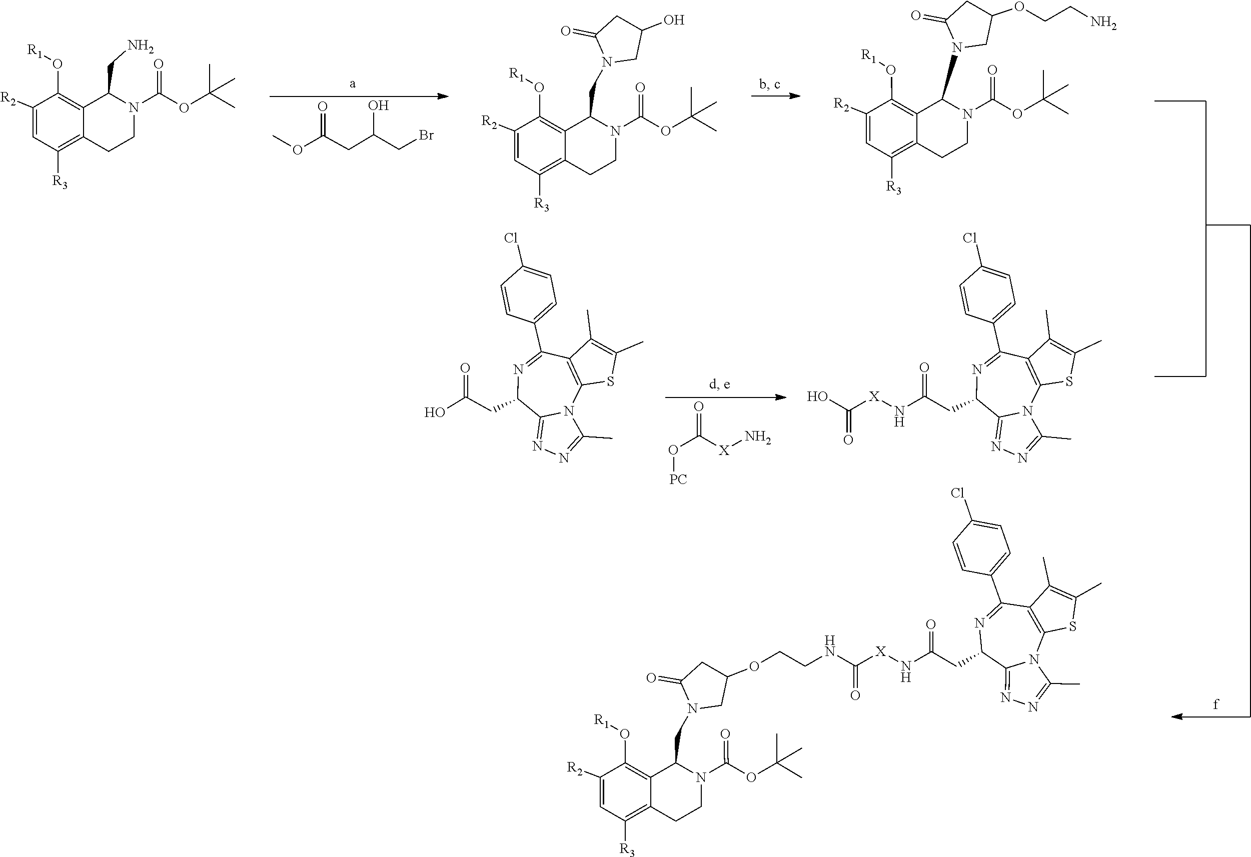

Scheme 7: a) toluene, heat; b) Cs₂CO₃, DMF, (9H-fluoren-9-yl)methyl (2-bromoethyl) carbamate; c) piperidine; d) HOAt, EDCl; e) acid deprotection; f) HOAt, EDCl
   General Method for PBG-L-KBG (-L¹-C(R⁶)(R⁷)-L²-C(R⁸)(R⁹)(R¹⁰) Attachment)

Compounds of the invention where the PBG-linker(L) is attached to the group -L¹-C(R⁶)(R⁷)-L²-C(R⁸)(R⁹)(R¹⁰) may be prepared from suitable intermediates prepared using the scheme below:

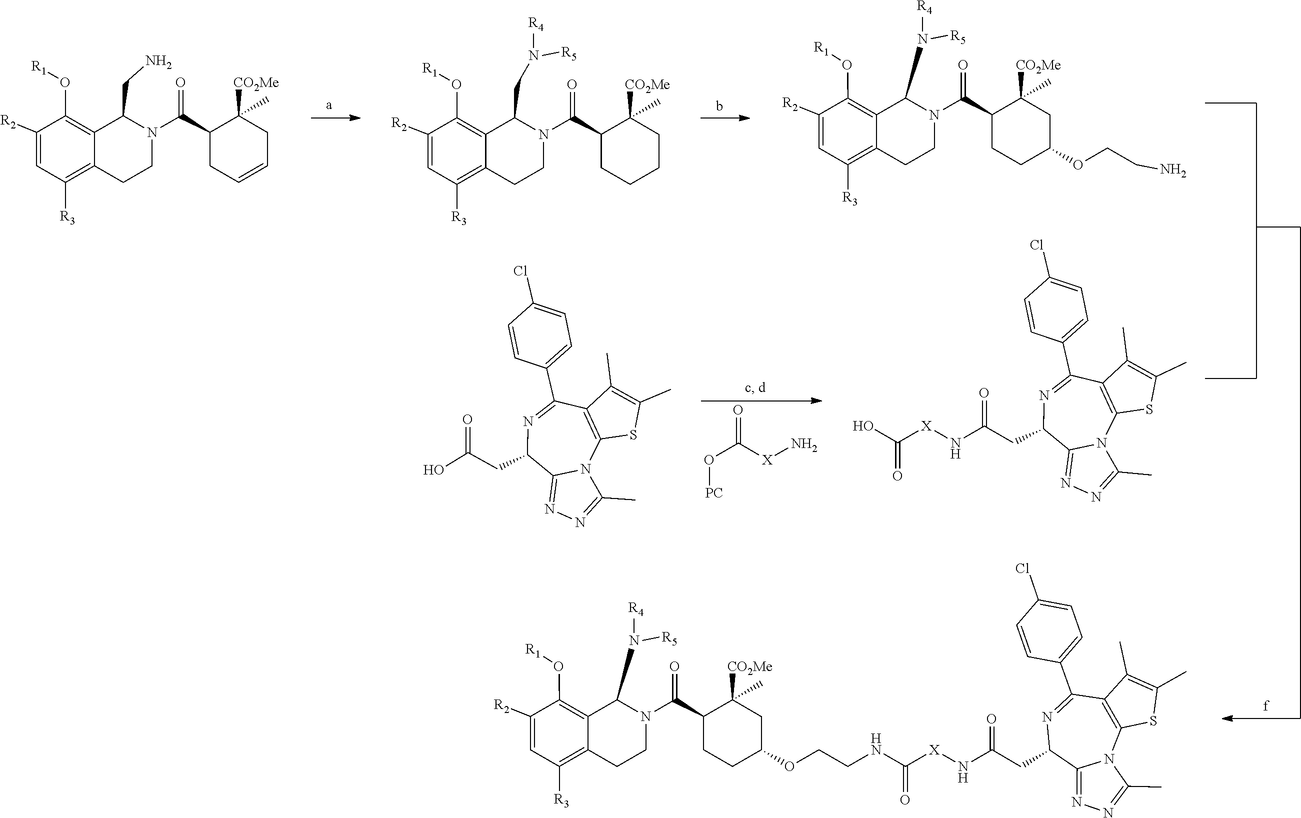

Scheme 8: a) BH₃.DMS, NaOH, H₂O₂; b) Cs₂CO₃, DMF, (9H-fluoren-9-yl)methyl (2-bromoethyl) carbamate, then piperidine, DMF; c) HOAt, EDCl; d) acid deprotection; e) HOAt, EDCl; f) TFA/DCM

### Pharmaceutical Compositions

The compounds of the invention will normally, but not necessarily, be formulated into pharmaceutical compositions prior to administration to a patient. Therefore, according to a further aspect of the invention there is provided a pharmaceutical composition which comprises a compound of Formula I as defined hereinbefore, or a pharmaceutically acceptable salt thereof, and one or more pharmaceutically acceptable excipients.

The pharmaceutical compositions of the invention may be prepared and packaged in bulk form wherein a safe and effective amount of a compound of the invention can be extracted and then given to the patient such as with powders or syrups. Alternatively, the pharmaceutical compositions of the invention may be prepared and packaged in unit dosage form wherein each physically discrete unit contains a safe and effective amount of a compound of the invention. When prepared in unit dosage form, the pharmaceutical compositions of the invention typically contain from 1 mg to 1000 mg.

The compositions of the invention may be in a form suitable for oral use (for example as tablets, capsules, caplets, pills, troches, powders, syrups, elixirs, suspensions, solutions, emulsions, sachets, and cachets), for topical use (for example as creams, ointments, lotions, solutions, pastes, sprays, foams, and gels), for transdermal administration such as via transdermal patches, for administration by inhalation (for example as a dry powders, aerosols, suspensions, and solutions), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

In a convenient embodiment, the pharmaceutical composition of the present invention is in a form suitable for parenteral administration, such as intravenous or intraperitoneal administration. Solutions or suspensions used for parenteral administration may comprise one or more of the following excipients: sterile diluent (e.g. water, saline, oils, glycerine, propylene glycol, polyethylene glycols, or other pharmaceutically acceptable solvents); buffer agent (e.g. acetates, citrates, phosphates, or tonicity adjusters such as sodium chloride or dextrose); chelating agent (e.g. edta); antibacterial agent (e.g. methyl parabens or benzyl alcohol); or antioxidant (e.g. ascorbic acid or sodium bisulfite). In a convenient embodiment, the pharmaceutical composition of the present invention is in a form suitable for intravenous or intraperitoneal administration and comprises phosphate buffered saline or physiological saline. Conveniently, the parenteral composition is enclosed within ampoules, vials, or syringes made from either glass or plastic.

As used herein, “pharmaceutically-acceptable excipient” means a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must be of sufficiently high purity to render it pharmaceutically-acceptable.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting of the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, hemectants, chelating agents, plasticizers, viscosity increasing agents, antioxidants, preservatives, stabilizers, surfactants, and buffering agents. The person skilled in the art will appreciate that certain pharmaceutically-acceptable excipients may serve more than one function and may serve alternative functions depending on how much of the excipient is present in the formulation and what other ingredients are present in the formulation.

Persons skilled in the art possess the knowledge and skill to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula I will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general, lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Routes of Administration

The compounds of the invention or pharmaceutical composition comprising the active compound may be administered to a subject by any convenient route of administration, whether systemically/peripherally or topically (i.e. at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

In a preferred embodiment, a compound of the invention as defined herein, or a pharmaceutically acceptable salt thereof, or a pharmaceutical composition as defined herein, is administered orally, intravenously, subcutaneously or intramuscularly.

### Therapeutic Uses and Applications

The compounds of the present invention are degraders of certain proteins that can be degraded through the KEAP1-CUL3 ligase. As a consequence, they are potentially useful therapeutic agents for the treatment of cancer (both solid and blood cancers), and auto-immune diseases.

Thus, in one aspect, the present invention relates to a compound of Formula I as defined herein, or a pharmaceutically acceptable salt thereof, or a pharmaceutical composition as defined herein, for use in therapy.

In an aspect, the present invention relates to a compound of Formula I as defined herein, or a pharmaceutically acceptable salt thereof, or a pharmaceutical composition as defined herein, for use in the treatment of a disease or disorder mediated by degradation of a protein of interest.

In an aspect, the present invention relates to a method of treating a disease or disorder mediated by degradation of a protein of interest, said method comprising administering to a subject in need of such treatment a therapeutically effective amount of a compound of Formula I as defined herein, or a pharmaceutically acceptable salt thereof, or a pharmaceutical composition as defined herein.

In an aspect, the present invention provides a compound of Formula I as defined herein, or a pharmaceutically acceptable salt thereof, or a pharmaceutical composition as defined herein, for use in degrading proteins susceptible to degradation by KEAP ligase.

Proteins susceptible to degradation by KEAP ligase may include:
i) a bromodomain and extra-terminal (BET) family protein (such as BRD3, BRD4 or BRD9);
ii) an androgen receptor;
iii) an estrogen receptor;
iv) BCL-XL;
v) IRAK4;
vi) STAT3;
vii) BTK;
viii) TRK; or
ix) FAK.

Suitably, the protein susceptible to degradation by KEAP ligase is a bromodomain and extra-terminal (BET) family protein (such as BRD3, BRD4 or BRD9), or FAK. Suitably, the protein susceptible to degradation by KEAP ligase is BRD4.

In an aspect, the present invention provides a method of degrading proteins susceptible to degradation by KEAP ligase in vivo, said method comprising administering an effective amount of a compound of Formula I, or a pharmaceutically acceptable salt thereof.

In an aspect, the present invention provides a compound of Formula I as defined herein, or a pharmaceutically acceptable salt thereof, or a pharmaceutical composition as defined herein, for use in degrading proteins susceptible to degradation by KEAP ligase, wherein the use comprises contacting a cell with an effective amount of a compound of Formula I as defined herein, or a pharmaceutically acceptable salt thereof.

In an aspect, the present invention provides a method of degrading proteins susceptible to degradation by KEAP ligase in vivo, said method comprising contacting a cell with an effective amount of a compound of Formula I as defined herein, or a pharmaceutically acceptable salt thereof.

In an aspect, the present invention relates to a compound of Formula I as defined herein, or a pharmaceutically acceptable salt thereof, or a pharmaceutical composition as defined herein, for use in the treatment of cancer or autoimmune diseases.

In an aspect, the present invention relates to a method of treating cancer or an autoimmune disease, said method comprising administering to a subject in need of such treatment a therapeutically effective amount of a compound of Formula I as defined herein, or a pharmaceutically acceptable salt thereof, or a pharmaceutical composition as defined herein.

The cancer to be treated may be a solid tumour (such as breast cancer, bowel cancer, colorectal cancer, lung cancer, liver cancer, bladder cancer, cervical cancer, hepatocellular carcinoma, squamous cell carcinoma, melanoma, glioma, head and neck cancer, sarcoma, pancreatic cancer or prostate cancer) or a blood cancer (such as leukaemia, acute myeloid leukaemia, acute lymphoblastic leukaemia, juvenile myelomonocytic leukaemia, multiple myeloma, lymphoma, B cell malignancies (such as non-Hodgkin lymphoma and chronic lymphocytic leukaemia) or diffuse large B-cell lymphoma). Examples of particular cancers that the compounds of Formula I and their pharmaceutically acceptable salts may be used to treat include, but are not limited to, any one of the following: prostate cancer, breast cancer, B cell malignancies, sarcomas, acute myeloid leukaemia, multiple myeloma, or lymphoma.

Examples of auto-immune diseases or disorders include rheumatoid arthritis, systemic lupus erythematosus, Graves' disease, autoimmune haemolytic anaemia, multiple sclerosis, Type 1 diabetes, Goodpasture's syndrome, Hashimoto's thyroiditis, Guillain-Barre syndrome, immune thrombocytopenic purpura, atherosclerosis, Crohn's disease, ulcerative colitis, inflammatory bowel disease, ankylosing spondylitis, seronegative spondyloarthropathies, autoimmune thyroiditis, Sjogren's syndrome, Hughes' syndrome, psoriasis, psoriatic arthritis, myasthenia gravis, thrombocytopenic purpura, Addison's disease, primary biliary cirrhosis, diffuse scleroderma, polymyositis, dermatomyositis, autoimmune hepatitis, autoimmune sclerosing cholangitis, limited scleroderma, autoimmune uveitis, acquired haemophilia, pernicious anaemia, pemphigus, pemphigoid and vitiligo.

In an aspect, the present invention provides a method of degrading proteins susceptible to degradation by KEAP ligase in vitro, said method comprising administering an effective amount of a compound of Formula I, or a pharmaceutically acceptable salt thereof.

In an aspect, the present invention provides a method of degrading proteins susceptible to degradation by KEAP ligase in vitro, said method comprising contacting a cell with an effective amount of a compound of Formula I as defined herein, or a pharmaceutically acceptable salt thereof.

### Examples

### General Procedures

Methods for preparing the compounds of this invention are illustrated in the following Examples. Starting materials are made according to procedures known in the art, or as illustrated herein, or are available commercially. Commercial reagents were used without further purification. Where no reaction temperature is included, the reaction was performed at ambient temperature which is typically 18-27° C.

Where compounds described in the invention are characterized by ¹H NMR spectroscopy, spectra were recorded on 400 MHz Bruker instruments. Where no temperature is included, the spectra were recorded at ambient temperature. Chemical shift values are expressed in parts per million (ppm). Where NMR spectra are complex due to the presence of interconverting isomers, approximate partial integrations of signals are reported, or characterisation for the major isomer only is reported. The following abbreviations are used for the multiplicity of the NMR signals: s=singlet, b=broad, t=triplet, q=quartet, m=multiplet, d=doublet.

### Analytical LCMS

Where compounds described in the invention are characterized by LCMS data, retention time and molecular weight are determined using the methods listed in the table below. In cases where molecular weights of compounds of the invention are outside the limit of detection (typically >1200 Da) the LCMS data for the major detected peak is reported (highest % peak area as detected by UV).

Method 1: Acquity UPLC H-Class (PDA, QDa and ELS). Column: Kinetex C18 (5 μm, 50×4.6 mm). Conditions: water containing 0.1% formic acid [eluent A], MeCN (containing 0.1% formic acid) [eluent B], flow rate 1.5 mL/min. Gradient: 5-95% B 0.25-1.5 min, hold at 95% B 1.5-2.25 min. Column temp 40° C.

Method 2: Acquity UPLC H-Class (PDA, QDa and ELS). Column: Kinetex Evo C18 (5 μm, 50×4.6 mm). Conditions: water containing 10 mM ammonium bicarbonate [eluent A], MeCN [eluent B], flow rate 1.5 mL/min. Gradient: 5-60% B 0.25-1.5 min, hold at 60% B 1.5-2.25 min. Column temp 40° C.

Method 3: Acquity UPLC+Waters DAD+Waters SQD2, single quadrupole UPLC-MS. Column: Acquity UPLC HSS C18 (1.8 μm, 100×2.1 mm). Conditions: water containing 0.1% formic acid [eluent A], MeCN (containing 0.1% (VN) formic acid) [eluent B], flow rate 0.4 mL/min. Gradient: 5-95% B 0.4-6.0 min, hold at 95% B 6.0-6.8 min. Column temp 40° C.

Method 4: Acquity UPLC+Waters DAD+Waters SQD2, single quadrupole UPLC-MS. Column: Acquity UPLC BEH C18 (1.7 μm, 100×2.1 mm). Conditions: water containing 10 mM ammonium bicarbonate [eluent A], MeCN [eluent B], flow rate 0.4 mL/min. Gradient: 5-95% B 0.4-6.0 min, hold at 95% B 6.0-6.8 min. Column temp 40° C.

### ABBREVIATIONS

DCM Dichloromethane
DEAD Diethyl azodicarboxylate
DIPEA N,N-Diisopropylethylamine
DMEM Dulbecco's Modified Eagle Medium
DMF N,N-Dimethylformamide
DMSO Dimethylsulfoxide
EDCl N-(3-Dimethylaminopropyl)-N′-ethylcarbodiimide hydrochloride
ELS Evaporative Light Scattering
EtOAc Ethyl acetate
EtOH Ethanol
FBS Fetal Bovine Serum
h Hour(s)
HATU N-[(Dimethylamino)-1H-1,2,3-triazolo-[4,5-b]pyridin-1-ylmethylene]-N-methylmethanaminium hexafluorophosphate N-oxide
HCl Hydrogen chloride

### INTERMEDIATES

### Intermediate 1: 2,4-Dimethoxybenzyl (1S,2R)-2-((S)-5-chloro-8-hydroxy-1-((6-oxo-5-azaspiro[2,4]heptan-5-yl)methyl)-1,2,3,4-tetrahydroisoquinoline-2-carbonyl)-1-methylcyclohexane-1-carboxylate

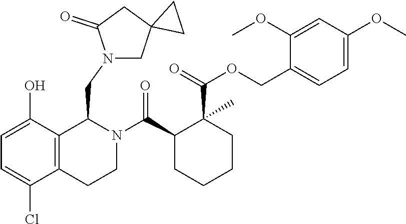 [SMILES: COc1ccc(COC(=O)[C@@]2(C)CCCC[C@H]2C(=O)N2CCc3c(Cl)ccc(O)c3[C@H]2CN2CC3(CC3)CC2=O)c(OC)c1]

Intermediate 1 is described in WO2020/084300 (Intermediate 37).

### Intermediate 2: tert-Butyl (S)-(2-(4-(4-chlorophenyl)-2,3,9-trimethyl-6H-thieno[3,2-f][1,2,4]triazolo[4,3-a][1,4]diazepin-6-yl)acetyl)glycinate

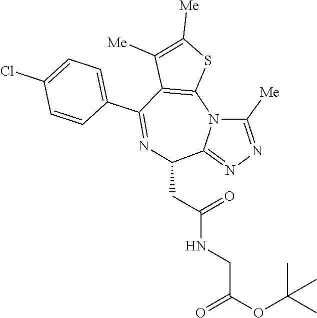 [SMILES: Cc1sc2c(c1C)C(c1ccc(Cl)cc1)=N[C@@H](CC(=O)NCC(=O)OC(C)(C)C)c1nnc(C)n1-2]

To a stirred solution of (S)-2-(4-(4-chlorophenyl)-2,3,9-trimethyl-6H-thieno[3,2-f][1,2,4]triazolo[4,3-a][1,4]diazepin-6-yl)acetic acid (1.0 g, 2.49 mmol; CAS: 202592-23-2), tert-butyl 2-aminoacetate hydrochloride (0.63 g, 3.74 mmol; CAS: 27532-96-3), 1-hydroxy-7-azabenzotriazole (0.51 g, 3.74 mmol; CAS: 39968-33-7) and EDC hydrochloride (0.72 g, 3.74 mmol) in DMSO (8.3 mL) was added 4-methylmorpholine (1.1 mL, 9.98 mmol; CAS: 109-02-4) and the reaction mixture was stirred at rt for 18 h. Purification by preparative HPLC (C18 120 g, 20-80% MeCN in water (10 mM ammonium bicarbonate)) gave the title compound (1.08 g, 80%). LCMS (Method 1): 1.70 min, 514.0 [M+H]⁺.

### Intermediate 3: (S)-(2-(4-(4-Chlorophenyl)-2,3,9-trimethyl-6H-thieno[3,2-f][1,2,4]triazolo[4,3-a][1,4]diazepin-6-yl)acetyl)glycine

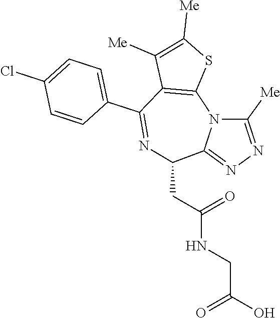 [SMILES: Cc1sc2c(c1C)C(c1ccc(Cl)cc1)=N[C@@H](CC(=O)NCC(=O)O)c1nnc(C)n1-2]

To a stirred solution of Intermediate 2 (1.08 g, 2.09 mmol) in DCM (4 mL) was added TFA (2.0 mL, 26.0 mmol) and the reaction mixture was stirred at rt for 2 h. A further portion of TFA (2.0 mL, 26.0 mmol) was added and the mixture stirred for another 2 h then concentrated in vacuo. The mixture was diluted with DCM and water, passed through a phase separator and the organics concentrated in vacuo. Purification by preparative HPLC (C18 120 g, 20-80% MeCN in water (10 mM ammonium bicarbonate)) gave the title compound (0.69 g, 72%). LCMS (Method 1): 1.43 min, 458.0 [M+H]⁺.

### Intermediate 4: tert-Butyl (S)-3-(2-(4-(4-chlorophenyl)-2,3,9-trimethyl-6H-thieno[3,2-f][1,2,4]triazolo[4,3-a][1,4]diazepin-6-yl)acetamido)propanoate

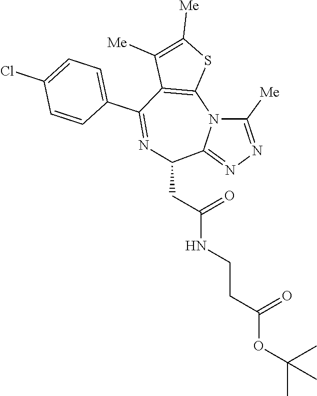 [SMILES: Cc1sc2c(c1C)C(c1ccc(Cl)cc1)=N[C@@H](CC(=O)NCCC(=O)OC(C)(C)C)c1nnc(C)n1-2]

To a stirred solution of (S)-2-(4-(4-chlorophenyl)-2,3,9-trimethyl-6H-thieno[3,2-f][1,2,4]triazolo[4,3-a][1,4]diazepin-6-yl)acetic acid (1.0 g, 2.49 mmol; CAS: 202592-23-2), tert-butyl 3-aminopropanoate (0.54 g, 3.74 mmol; CAS: 15231-41-1), 1-hydroxy-7-azabenzotriazole (0.51 g, 3.74 mmol; CAS: 39968-33-7) and EDC hydrochloride (0.72 g, 3.74 mmol) in DMSO (8.3 mL) was added 4-methylmorpholine (0.82 mL, 7.48 mmol; CAS: 109-02-4) and the reaction mixture was stirred at rt for 18 h. Purification by preparative HPLC (C18 120 g, 20-80% MeCN in water (10 mM ammonium bicarbonate)) gave the title compound (899 mg, 68%). LCMS (Method 1): 1.72 min, 528.0 [M+H]⁺.

### Intermediate 5: (S)-3-(2-(4-(4-Chlorophenyl)-2,3,9-trimethyl-6H-thieno[3,2-f][1,2,4]triazolo[4,3-a][1,4]diazepin-6-yl)acetamido)propanoic acid

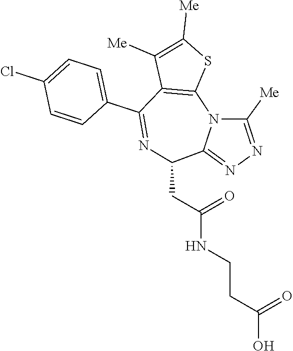 [SMILES: Cc1sc2c(c1C)C(c1ccc(Cl)cc1)=N[C@@H](CC(=O)NCCC(=O)O)c1nnc(C)n1-2]

To a stirred solution of Intermediate 4 (0.40 g, 0.76 mmol) in DCM (2.5 mL) was added TFA (0.7 mL, 9.4 mmol) and the reaction mixture was stirred at rt for 72 h then concentrated in vacuo. The mixture was diluted with DCM and water, passed through a phase separator and the organics concentrated in vacuo. Purification by preparative HPLC (C18 120 g, 20-80% MeCN in water (10 mM ammonium bicarbonate)) gave the title compound (0.26 g, 72%). LCMS (Method 1): 1.44 min, 472.0 [M+H]⁺.

### Intermediate 6: tert-Butyl (S)-3-(2-(2-(4-(4-chlorophenyl)-2,3,9-trimethyl-6H-thieno[3,2-f][1,2,4]triazolo[4,3-a][1,4]diazepin-6-yl)acetamido)ethoxy)propanoate

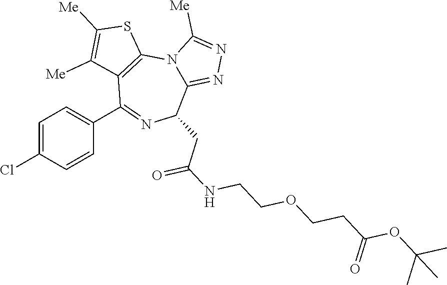 [SMILES: Cc1sc2c(c1C)C(c1ccc(Cl)cc1)=N[C@@H](CC(=O)NCCOCCC(=O)OC(C)(C)C)c1nnc(C)n1-2]

To a stirred solution of (S)-2-(4-(4-chlorophenyl)-2,3,9-trimethyl-6H-thieno[3,2-f][1,2,4]triazolo[4,3-a][1,4]diazepin-6-yl)acetic acid (0.40 g, 1.0 mmol; CAS: 202592-23-2) and tert-butyl 3-(2-aminoethoxy)propanoate (0.19 g, 1.0 mmol; CAS: 1260092-46-3) in DMF (3.3 mL) was added DIPEA (0.35 ml, 2.0 mmol) and HATU (0.42 g, 1.1 mmol; CAS: 148893-10-1) and the reaction mixture was stirred at rt for 18 h. The reaction mixture was concentrated in vacuo and purified by preparative HPLC (C18 120 g, 20-80% MeCN in water (10 mM ammonium bicarbonate)) gave the title compound (0.29 g, 51%). LCMS (Method 1): 1.72 min, 572.2 [M+H]1.

### Intermediate 7: (S)-3-(2-(2-(4-(4-Chlorophenyl)-2,3,9-trimethyl-6H-thieno[3,2-f][1,2,4]triazolo[4,3-a][1,4]diazepin-6-yl)acetamido)ethoxy)propanoic acid

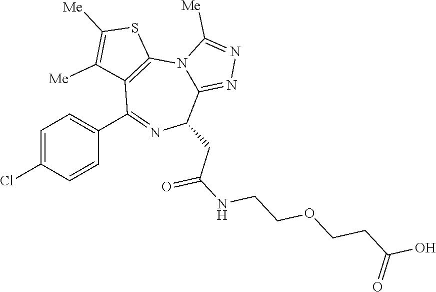 [SMILES: Cc1sc2c(c1C)C(c1ccc(Cl)cc1)=N[C@@H](CC(=O)NCCOCCC(=O)O)c1nnc(C)n1-2]

To a stirred solution of Intermediate 6 (290 mg, 0.51 mmol) in DCM (2.5 mL) was added TFA (2.0 mL, 26 mmol) and the reaction mixture was stirred at rt for 18 h then concentrated in vacuo. The mixture was diluted with DCM and water, passed through a phase separator and the organics concentrated in vacuo. Purification by preparative HPLC (C18 120 g, 20-80% MeCN in water (10 mM ammonium bicarbonate)) gave the title compound (0.20 g, 76%). LCMS (Method 1): 1.45 min, 516.5 [M+H]⁺.

### Intermediate 8: tert-Butyl (R)-(2-(2-(3-hydroxypyrrolidin-1-yl)-2-oxoethoxy)-ethyl)carbamate

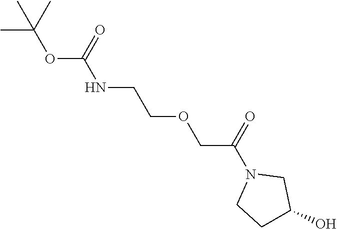 [SMILES: CC(C)(C)OC(=O)NCCOCC(=O)N1CC[C@@H](O)C1]

To a stirred solution of 2-(2-((tert-butoxycarbonyl)amino)ethoxy)acetic acid (1.26 g, 5.74 mmol; CAS: 142929-49-5) and (R)-pyrrolidin-3-ol (0.48 mL, 5.74 mmol; CAS: 2799-21-5) in DCM (19 mL) was added DIPEA (2.0 mL, 11.5 mmol) and HATU (2.40 g, 6.31 mmol) and the reaction mixture stirred at rt for 18 h. The mixture was diluted with DCM, washed with sodium hydroxide (1 M aqueous), the combined organics were passed through a phase separator and concentrated in vacuo to give the title compound (1.35 g, 82%), used without further purification. LCMS (Method 1): 1.17 min, 289.1 [M+H]⁺.

### Intermediate 9: (R)-2-(2-Aminoethoxy)-1-(3-hydroxypyrrolidin-1-yl)ethan-1-one

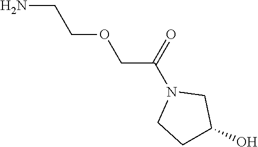 [SMILES: NCCOCC(=O)N1CC[C@@H](O)C1]

To a stirred solution of Intermediate 8 (1.25 g, 4.34 mmol) in DCM (9 mL) was added TFA (8.0 mL, 104 mmol) and the reaction mixture was stirred at rt for 24 h. The mixture was concentrated in vacuo, diluted with DCM/MeOH (1:1) and purified by SCX cartridge (washed with DCM/MeOH (1:1) and eluted with DCM/methanolic ammonia (1:1; 7N NH₃)) to give the title compound (449 mg, 55%), used without further purification. LCMS (Method 1): 1.01 min, 189.1 [M+H]⁺.

### Intermediate 10: 2-((S)-4-(4-Chlorophenyl)-2,3,9-trimethyl-6H-thieno[3,2-f][1,2,4]triazolo[4,3-a][1,4]diazepin-6-yl)-N-(2-((2-(2-((R)-3-hydroxypyrrolidin-1-yl)-2-oxoethoxy)ethyl)amino)-2-oxoethyl)acetamide

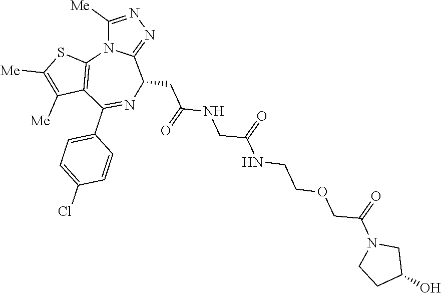 [SMILES: Cc1sc2c(c1C)C(c1ccc(Cl)cc1)=N[C@@H](CC(=O)NCC(=O)NCCOCC(=O)N1CC[C@@H](O)C1)c1nnc(C)n1-2]

To a stirred solution of (S)-2-(4-(4-chlorophenyl)-2,3,9-trimethyl-6H-thieno[3,2-f][1,2,4]triazolo[4,3-a][1,4]diazepin-6-yl)acetic acid (0.46 g, 1.01 mmol; CAS: 202592-23-2) and Intermediate 9 (0.19 g, 1.01 mmol) in DMF (3.4 mL) was added DIPEA (0.35 mL, 2.0 mmol) and HATU (0.42 g, 1.11 mmol) and the reaction mixture was stirred at rt for 18 h. The reaction mixture was concentrated in vacuo and purified by preparative HPLC (C18 120 g, 20-80% MeCN (0.1% formic acid)/H₂O (0.1% formic acid)) to give the title compound (0.34 g, 38%). LCMS (Method 1): 1.33 min, 628.3 [M+H]⁺.

### Intermediate 11: 2-(2-(4-(Chloromethyl)-5-methyl-1H-1,2,3-triazol-1-yl)ethyl)isoindoline-1,3-dione

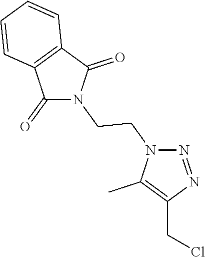 [SMILES: Cc1c(CCl)nnn1CCN1C(=O)c2ccccc2C1=O]

To a stirred solution of 2-[2-[4-(hydroxymethyl)-5-methyl-triazol-1-yl]ethyl]isoindoline-1,3-dione (0.2 g, 0.7 mmol, CAS: 1955526-81-4) and triethylamine (0.15 mL, 1.05 mmol) in DCM (3.5 mL) was added a solution of methanesulfonyl chloride (0.06 mL, 0.84 mmol, CAS: 124-63-0) and the mixture was stirred at rt for 18 h. To this was added saturated aqueous NaHCO₃ and the mixture was extracted with DCM. The combined organics were passed through a phase separator and concentrated in vacuo to provide the title compound (0.24 g, 98%) used without further purification. LCMS (Method 1): 1.38 min, 305.3 [M+H]⁺.

### Intermediate 12: 2,4-Dimethoxybenzyl (1S,2R)-2-((S)-5-chloro-8-((1-(2-(1,3-dioxoisoindolin-2-yl)ethyl)-5-methyl-1H-1,2,3-triazol-4-yl)methoxy)-1-((6-oxo-5-azaspiro[2,4]heptan-5-yl)methyl)-1,2,3,4-tetrahydroisoquinoline-2-carbonyl)-1-methylcyclohexane-1-carboxylate

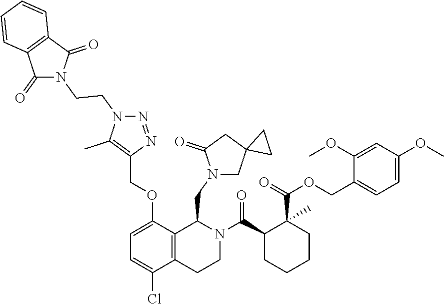 [SMILES: COc1ccc(COC(=O)[C@@]2(C)CCCC[C@H]2C(=O)N2CCc3c(Cl)ccc(OCc4nnn(CCN5C(=O)c6ccccc6C5=O)c4C)c3[C@H]2CN2CC3(CC3)CC2=O)c(OC)c1]

To a stirred solution of Intermediate 11 (0.23 g, 0.77 mmol) in DMF (1.0 mL) was added Intermediate 1 (0.32 g, 0.51 mmol) and caesium carbonate (0.33 g, 1.02 mmol) and the reaction mixture stirred at rt under nitrogen for 7 days. The mixture was diluted with EtOAc, and LiCl (4% aqueous) and the aqueous layer was re-extracted with EtOAc. The combined organics were dried over MgSO₄, passed through a phase separator and concentrated in vacuo. Purification by flash column chromatography (Biotage Isolera™, 25 g silica column, eluting 0-50% (EtOAc:EtOH 3:1) in cyclohexane) provided the title compound (0.33 g, 65%). LCMS (Method 1): 1.86 min, 894.0 [M+H]⁺.

### Intermediate 13: 2,4-Dimethoxybenzyl (1S,2R)-2-((S)-8-((1-(2-aminoethyl)-5-methyl-1H-1,2,3-triazol-4-yl)methoxy)-5-chloro-1-((6-oxo-5-azaspiro[2,4]heptan-5-yl)methyl)-1,2,3,4-tetrahydroisoquinoline-2-carbonyl)-1-methylcyclohexane-1-carboxylate

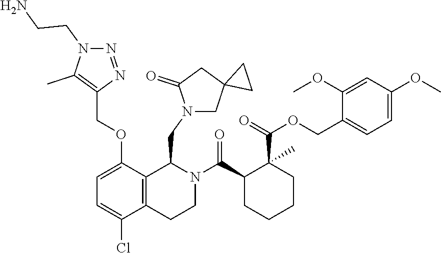 [SMILES: COc1ccc(COC(=O)[C@@]2(C)CCCC[C@H]2C(=O)N2CCc3c(Cl)ccc(OCc4nnn(CCN)c4C)c3[C@H]2CN2CC3(CC3)CC2=O)c(OC)c1]

To a stirred suspension of Intermediate 12 (0.33 g, 0.33 mmol) in EtOH (3.3 mL) was added hydrazine monohydrate (0.06 mL, 0.83 mmol, CAS: 7803-57-8) and the reaction mixture heated to 75° C. and stirred for 4 h. This was cooled to rt, diluted with additional EtOH and MeCN and filtered to remove the phthalhydrazide by-product. The filter cake was washed with additional EtOH and MeCN and the combined filtrates concentrated in vacuo to give the title compound (0.25 g, assumed quantitative), used without further purification. LCMS (Method 1): 1.79 min, 763.9 [M+H]⁺.

### Intermediate 14: 2,4-Dimethoxybenzyl (1S,2R)-2-((S)-5-chloro-8-((1-(2-(2-(2-((S)-4-(4-chlorophenyl)-2,3,9-trimethyl-6H-thieno[3,2-f][1,2,4]triazolo[4,3-a][1,4]diazepin-6-yl)acetamido)acetamido)ethyl)-5-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylate

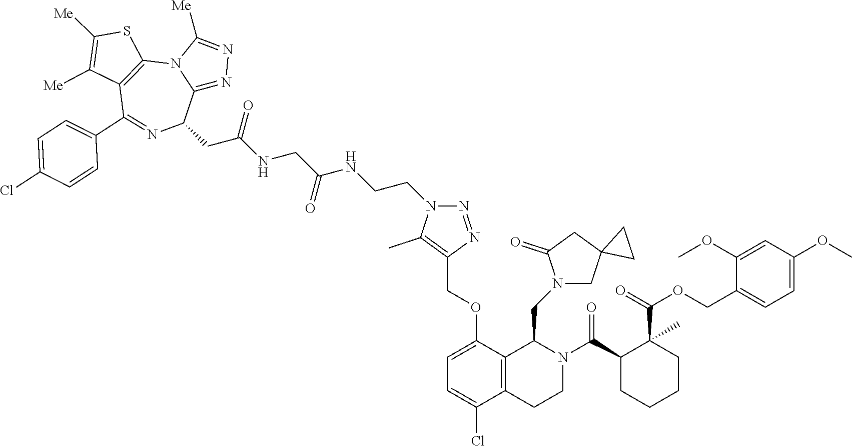 [SMILES: COc1ccc(COC(=O)[C@@]2(C)CCCC[C@H]2C(=O)N2CCc3c(Cl)ccc(OCc4nnn(CCNC(=O)CNC(=O)C[C@@H]5N=C(c6ccc(Cl)cc6)c6c(sc(C)c6C)-n6c(C)nnc65)c4C)c3[C@H]2CN2CC3(CC3)CC2=O)c(OC)c1]

To a stirred solution of Intermediate 3 (0.17 g, 0.36 mmol) and Intermediate 13 (0.25 g, 0.33 mmol) in DMF (2.2 mL) was added DIPEA (0.17 mL, 0.99 mmol), followed by HATU (0.15 g, 0.4 mmol) and the mixture was stirred at rt for 16 h. Purification by preparative HPLC (C18 120 g, 40-100% MeCN in water (10 mM ammonium bicarbonate)) gave the title compound (0.18 mg, 47%). LCMS (Method 1): 1.93 min, 1202.5 [M+H]⁺.

### Intermediate 15: 5-(((tert-Butyldiphenylsilyl)oxy)methyl)-4-(chloromethyl)-1-methyl-1H-1,2,3-triazole

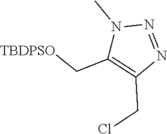

To a stirred solution of (5-(((tert-butyldiphenylsilyl)oxy)methyl)-1-methyl-1H-1,2,3-triazol-4-yl)methanol (0.53 g, 1.40 mmol; CAS: 432554-87-5, WO2002042305) and triethylamine (0.29 mL, 2.1 mmol) in DCM (5.6 mL) was added methanesulfonyl chloride (0.13 mL, 1.68 mmol) and the reaction mixture was stirred at rt for 48 h. To this was added saturated aqueous NaHCO₃ and the mixture was extracted with DCM. The combined organics were passed through a phase separator and concentrated in vacuo to provide the title compound (0.63 g, assumed quantitative), used without further purification. LCMS (Method 1): 1.95 min, 400.5 [M+H]⁺.

### Intermediate 16: 2,4-Dimethoxybenzyl (1S,2R)-2-((S)-8-((5-(((tert-butyldiphenylsilyl)oxy)methyl)-1-methyl-1H-1,2,3-triazol-4-yl)methoxy)-5-chloro-1-((6-oxo-5-azaspiro[2.4]heptan-5-yl)methyl)-1,2,3,4-tetrahydroisoquinoline-2-carbonyl)-1-methylcyclohexane-1-carboxylate

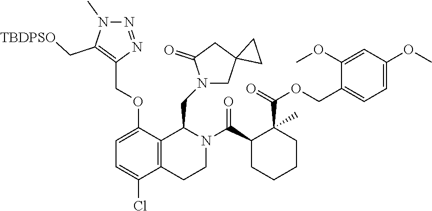

To a stirred solution of Intermediate 15 (0.63 g, 1.58 mmol) in DMF (6.6 mL) was added Intermediate 1 (0.83 mg, 1.32 mmol) and caesium carbonate (1.29 g, 3.96 mmol) and the reaction mixture stirred at rt under nitrogen for 16 h. The mixture was diluted with EtOAc, and LiCl (4% aqueous) and the aqueous layer was re-extracted with EtOAc. The combined organics were dried over MgSO₄, passed through a phase separator and concentrated in vacuo to provide the title compound (1.22 g, 94%), used without further purification. LCMS (Method 1): 2.19 min, 989.2 [M+H]⁺.

### Intermediate 17: 2,4-Dimethoxybenzyl (1S,2R)-2-((S)-5-chloro-8-((5-(hydroxymethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylate

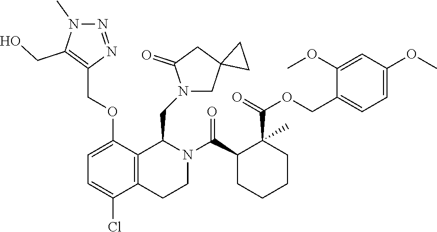 [SMILES: COc1ccc(COC(=O)[C@@]2(C)CCCC[C@H]2C(=O)N2CCc3c(Cl)ccc(OCc4nnn(C)c4CO)c3[C@H]2CN2CC3(CC3)CC2=O)c(OC)c1]

To a stirred solution of Intermediate 16 (1.22 g, 1.23 mmol) in THF (10 mL) and MeOH (2 mL) was added TBAF (1.23 mL, 1.23 mmol; 1M in THF) and the reaction mixture was stirred at rt for 72 h. The mixture was diluted with water and extracted with EtOAc and the combined organics washed with brine, passed through a phase separator and concentrated in vacuo. Purification by flash column chromatography (40 g silica column, eluted with 0-50% [EtOAc:EtOH=3:1] in cyclohexanes) provided the title compound (0.58 g, 50%). LCMS (Method 2): 1.72 min, 750.4 [M+H]⁺.

### Intermediate 18: 2,4-Dimethoxybenzyl (1S,2R)-2-((S)-5-chloro-8-((5-(chloromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylate

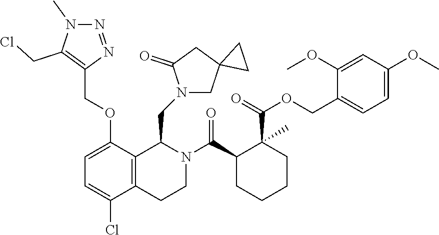 [SMILES: COc1ccc(COC(=O)[C@@]2(C)CCCC[C@H]2C(=O)N2CCc3c(Cl)ccc(OCc4nnn(C)c4CCl)c3[C@H]2CN2CC3(CC3)CC2=O)c(OC)c1]

To a stirred solution of Intermediate 17 (0.25 g, 0.33 mmol) and triethylamine (0.08 mL, 0.6 mmol) in DCM (2.2 mL) was added methanesulfonyl chloride (0.04 mL, 0.5 mmol) and the mixture was stirred at rt for 16 h. To this was added saturated aqueous NaHCO₃ and the mixture was extracted with DCM. The combined organics were passed through a phase separator and concentrated in vacuo to provide the title compound (0.23 g, 91%), used without further purification. LCMS (Method 1): 1.83 min, 768.8 [M+H]⁺.

### Intermediate 19: 2,4-Dimethoxybenzyl (1S,2R)-2-((S)-5-chloro-8-((5-((2-(1,3-dioxoisoindolin-2-yl)ethoxy)methyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylate

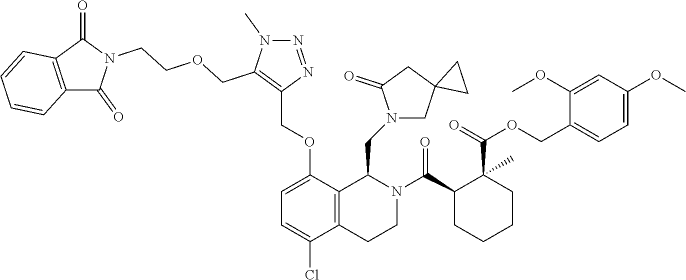 [SMILES: COc1ccc(COC(=O)[C@@]2(C)CCCC[C@H]2C(=O)N2CCc3c(Cl)ccc(OCc4nnn(C)c4COCCN4C(=O)c5ccccc5C4=O)c3[C@H]2CN2CC3(CC3)CC2=O)c(OC)c1]

To a stirred solution of 2-(2-hydroxyethyl)isoindoline-1,3-dione (60 mg, 0.31 mmol, CAS: 3891-07-4) in anhydrous DMF (0.8 mL) was added sodium hydride (14 mg, 0.36 mmol; 60% dispersion in mineral oil), and the suspension was stirred for 15 min. To this was added a solution of Intermediate 18 (0.18 g, 0.24 mmol) in DMF (0.8 mL), and the reaction mixture was heated to 60° C. for 3.5 h. The reaction was diluted with water and partitioned between DCM and LiCl solution (4% aqueous). The aqueous phase was further extracted, the combined organics were passed through a phase separator and concentrated in vacuo to give the title compound (0.18 g, 41%), used without further purification. LCMS (Method 2): 1.87 min, 923.5 [M+H]⁺.

### Intermediate 20: 2,4-Dimethoxybenzyl (1S,2R)-2-((S)-8-((5-((2-aminoethoxy)methyl)-1-methyl-1H-1,2,3-triazol-4-yl)methoxy)-5-chloro-1-((6-oxo-5-azaspiro[2.4]heptan-5-yl)methyl)-1,2,3,4-tetrahydroisoquinoline-2-carbonyl)-1-methylcyclohexane-1-carboxylate

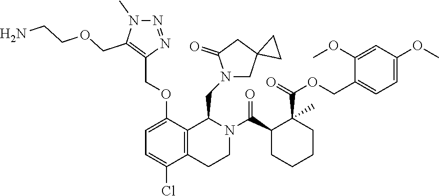 [SMILES: COc1ccc(COC(=O)[C@@]2(C)CCCC[C@H]2C(=O)N2CCc3c(Cl)ccc(OCc4nnn(C)c4COCCN)c3[C@H]2CN2CC3(CC3)CC2=O)c(OC)c1]

To a stirred suspension of Intermediate 19 (0.18 g, 0.2 mmol) in EtOH (2.0 mL) was added hydrazine monohydrate (0.04 mL, 0.49 mmol) and the resulting mixture heated to 75° C. and stirred for 16 h. The reaction mixture was cooled to rt, diluted with additional EtOH and MeCN and filtered to remove the phthalhydrazide by-product. The filter cake was washed with additional EtOH and MeCN and the combined filtrates concentrated in vacuo to give the title compound (0.19 g, 68%) used without further purification. LCMS (Method 2): 1.81 min, 793.5 [M+H]⁺.

### Intermediate 21: 2,4-Dimethoxybenzyl (1S,2R)-2-((S)-5-chloro-8-((5-((2-(2-(2-((S)-4-(4-chlorophenyl)-2,3,9-trimethyl-6H-thieno[3,2-f][1,2,4]triazolo[4,3-a][1,4]diazepin-6-yl)acetamido)acetamido)ethoxy)methyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylate

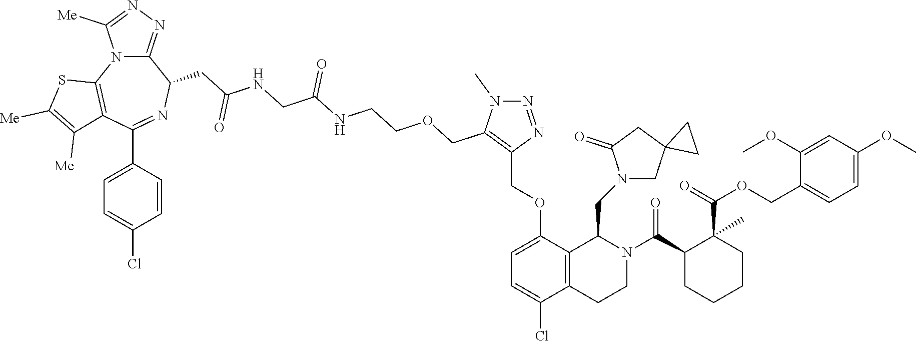 [SMILES: COc1ccc(COC(=O)[C@@]2(C)CCCC[C@H]2C(=O)N2CCc3c(Cl)ccc(OCc4nnn(C)c4COCCNC(=O)CNC(=O)C[C@@H]4N=C(c5ccc(Cl)cc5)c5c(sc(C)c5C)-n5c(C)nnc54)c3[C@H]2CN2CC3(CC3)CC2=O)c(OC)c1]

To a stirred solution of Intermediate 3 (0.14 g, 0.3 mmol) and Intermediate 20 (0.19 g, 0.23 mmol) in DMF (2.2 mL) was added DIPEA (0.12 mL, 0.7 mmol) and HATU (0.13 g, 0.33 mmol) and the mixture was stirred at rt for 18 h. Purification by preparative HPLC (C18 120 g, 40-100% MeCN in water (10 mM ammonium bicarbonate)) gave the title compound (98 mg, 30%). LCMS (Method 2): 1.85 min, 1082.5 [M-DMB+2H]⁺.

### Intermediate 22: 2,4-Dimethoxybenzyl (1S,2R)-2-((S)-5-chloro-8-((5-((2-(3-(2-((S)-4-(4-chlorophenyl)-2,3,9-trimethyl-6H-thieno[3,2-f][1,2,4]triazolo[4,3-a][1,4]diazepin-6-yl)acetamido)propanamido)ethoxy)methyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylate

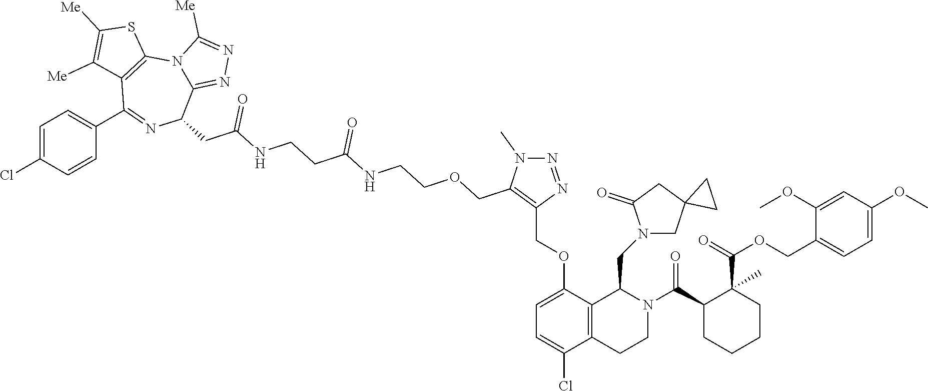 [SMILES: COc1ccc(COC(=O)[C@@]2(C)CCCC[C@H]2C(=O)N2CCc3c(Cl)ccc(OCc4nnn(C)c4COCCNC(=O)CCNC(=O)C[C@@H]4N=C(c5ccc(Cl)cc5)c5c(sc(C)c5C)-n5c(C)nnc54)c3[C@H]2CN2CC3(CC3)CC2=O)c(OC)c1]

To a stirred solution of Intermediate 5 (0.13 g, 0.27 mmol) and Intermediate 20 (0.14 g, 0.18 mmol) in DMF (2.2 mL) was added DIPEA (0.12 mL, 0.72 mmol) and HATU (0.14 g, 0.38 mmol) and the mixture was stirred at rt for 16 h. Purification by preparative HPLC (C18 120 g, 40-100% MeCN in water (10 mM ammonium bicarbonate)) gave the title compound (69 mg, 31%). LCMS (Method 2): 1.82 min, 1096.5 [M-DMB+2H]⁺.

### Intermediate 23: 2,4-Dimethoxybenzyl (1S,2R)-2-((S)-5-chloro-8-((5-(14-((S)-4-(4-chlorophenyl)-2,3,9-trimethyl-6H-thieno[3,2-f][1,2,4]triazolo[4,3-a][1,4]diazepin-6-yl)-6,13-dioxo-2,9-dioxa-5,12-diazatetradecyl)-1-methyl-1H-1,2,3-triazol-4-yl)methoxy)-1-((6-oxo-5-azaspiro[2.4]heptan-5-yl)methyl)-1,2,3,4-tetrahydroisoguinoline-2-carbonyl)-1-methylcyclohexane-1-carboxylate

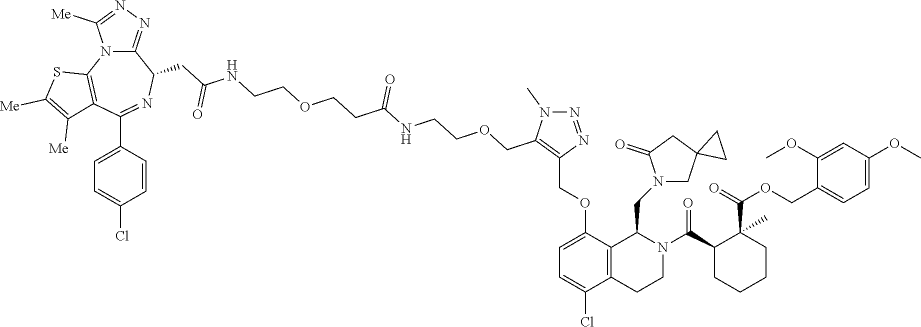 [SMILES: COc1ccc(COC(=O)[C@@]2(C)CCCC[C@H]2C(=O)N2CCc3c(Cl)ccc(OCc4nnn(C)c4COCCNC(=O)CCOCCNC(=O)C[C@@H]4N=C(c5ccc(Cl)cc5)c5c(sc(C)c5C)-n5c(C)nnc54)c3[C@H]2CN2CC3(CC3)CC2=O)c(OC)c1]

To a stirred solution of Intermediate 7 (0.19 g, 0.37 mmol) and Intermediate 20 (0.14 g, 0.18 mmol) in DMF (1.8 mL) was added DIPEA (0.12 mL, 0.72 mmol) and HATU (0.14 g, 0.38 mmol) and the mixture stirred at rt for 16 h. Purification by preparative HPLC (C18 120 g, 40-100% MeCN in water (10 mM ammonium bicarbonate)) gave the title compound (38 mg, 13%). LCMS (Method 2): 1.82 min, 1140.6 [M-DMB+2H]⁺.

### Intermediate 24: 2,4-Dimethoxybenzyl (1S,2R)-2-((S)-5-chloro-8-(((S)-1-(2-(2-(2-(2-((S)-4-(4-chlorophenyl)-2,3,9-trimethyl-6H-thieno[3,2-f][1,2,4]triazolo[4,3-a][1,4]diazepin-6-yl)acetamido)acetamido)ethoxy)acetyl)pyrrolidin-3-yl)oxy)-1-((6-oxo-5-azaspiro[2.4]heptan-5-yl)methyl)-1,2,3,4-tetrahydroisoquinoline-2-carbonyl)-1-methylcyclohexane-1-carboxylate

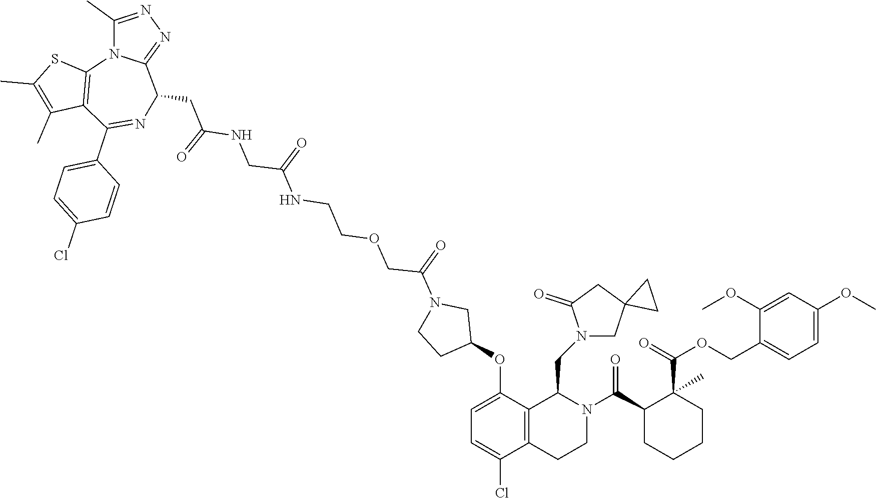

To a stirred solution of Intermediate 10 (0.14 g, 0.22 mmol) and Intermediate 1 (0.14 g, 0.22 mmol) in THF (1.5 mL) was added triphenylphosphine (64 mg, 0.25 mmol; CAS: 603-35-0) and DEAD (0.11 mL, 0.25 mmol; 40% in toluene, CAS: 1972-28-7) and the reaction mixture stirred at rt for 18 h. The reaction mixture was heated to 50° C. for 5 h, cooled to rt and further triphenylphosphine (64 mg, 0.25 mmol; CAS: 603-35-0) and DEAD (0.11 mL, 0.25 mmol; 40% in toluene, CAS: 1972-28-7) were added. The reaction mixture was heated to 60° C. for 16 h, cooled to rt and concentrated in vacuo. Purification by preparative HPLC (C18, 40-100% MeCN in water (10 mM ammonium bicarbonate)) provided the title compound (27 mg, 2%). LCMS (Method 2): 1.78 min, 1084.4 [M-DMB+2H]⁺.

### Example 1: (1S,2R)-2-((S)-5-Chloro-8-((1-(2-(2-(2-((S)-4-(4-chlorophenyl)-2,3,9-trimethyl-6H-thieno[3,2-f][1,2,4]triazolo[4,3-a][1,4]diazepin-6-yl)acetamido)acetamido)ethyl)-5-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid

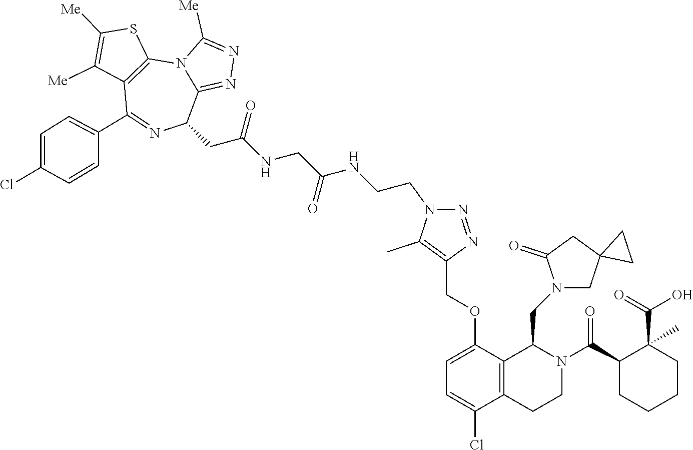 [SMILES: Cc1sc2c(c1C)C(c1ccc(Cl)cc1)=N[C@@H](CC(=O)NCC(=O)NCCn1nnc(COc3ccc(Cl)c4c3[C@@H](CN3CC5(CC5)CC3=O)N(C(=O)[C@@H]3CCCC[C@]3(C)C(=O)O)CC4)c1C)c1nnc(C)n1-2]

To a stirred solution of Intermediate 14 (0.18 g, 0.15 mmol) and triethylsilane (0.19 mL, 1.2 mmol, CAS: 617-86-7) in DCM (1.5 mL) at rt was added TFA (0.05 mL, 0.6 mmol) and the reaction mixture stirred at rt for 1 h. The mixture was concentrated in vacuo, the residue was diluted with EtOAc (100 mL) and the organics washed with water. The combined organics were passed through a phase separator, dried (Na₂SO₄) and concentrated in vacuo. Purification by preparative HPLC (C18 120 g, 20-80% MeCN in water (10 mM ammonium bicarbonate)) gave the title compound (60 mg, 38%). LCMS (Method 3): 5.02 min, 1052.8 [M+H]⁺. ¹H NMR (400 MHz, DMSO-d₆) δ 12.05-11.52 (bm, 1H), 8.74-8.60 (m, 1H), 8.19-8.04 (m, 1H), 7.52-7.42 (m, 4H), 7.36 (d, 1H), 7.13 (d, 1H), 5.80-5.73 (m, 1H), 5.16-5.06 (m, 2H), 4.52 (t, 1H), 4.34 (t, 2H), 4.03-3.87 (m, 2H), 3.81 (dd, 1H), 3.66-3.44 (m, 4H), 3.43-3.23 (m, 3H), 3.06-2.94 (m, 2H), 2.85-2.76 (m, 1H), 2.75-2.64 (m, 2H), 2.61 (s, 3H), 2.43-2.22 (m, 7H), 2.19 (d, 1H), 2.09 (d, 1H), 1.73-1.47 (m, 6H), 1.45-1.18 (m, 4H), 1.10 (s, 3H), 0.60-0.38 (m, 4H).

### Example 2: (1S,2R)-2-((S)-5-Chloro-8-((1-(2-(2-(2-((S)-4-(4-chlorophenyl)-2,3,9-trimethyl-6H-thieno[3,2-f][1,2,4]triazolo[4,3-a][1,4]diazepin-6-yl)acetamidolacetamido)ethyl)-5-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-N,1-dimethylcyclohexane-1-carboxamide

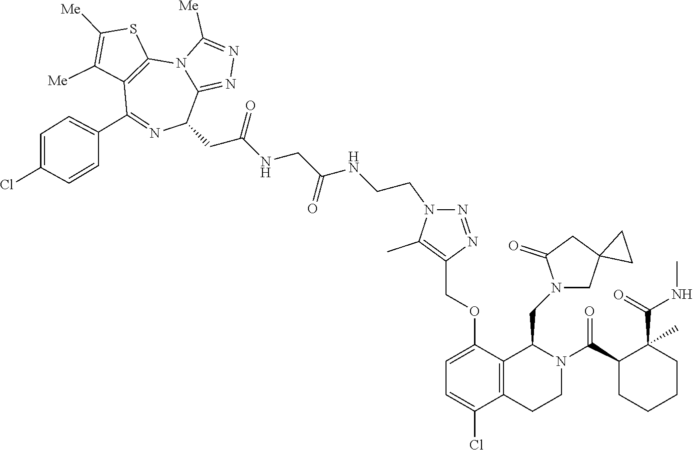 [SMILES: CNC(=O)[C@@]1(C)CCCC[C@H]1C(=O)N1CCc2c(Cl)ccc(OCc3nnn(CCNC(=O)CNC(=O)C[C@@H]4N=C(c5ccc(Cl)cc5)c5c(sc(C)c5C)-n5c(C)nnc54)c3C)c2[C@H]1CN1CC2(CC2)CC1=O]

To a stirred suspension of HATU (6.5 mg, 0.02 mmol) and DIPEA (0.02 mL, 0.02 mmol) in DMF (0.2 mL) was added Example 1 (15 mg, 0.01 mmol) and methylamine solution (0.02 mL, 0.03 mmol; 2.0 M in THF, CAS: 74-89-5) and the reaction mixture was stirred at rt for 18 h. Additional methylamine solution (0.1 mL, 0.2 mmol; 2.0 M in THF, CAS: 74-89-5), DIPEA (0.02 mL, 0.11 mmol) and HATU (40 mg, 0.11 mmol) were added and stirred for 72 h. The reaction mixture was concentrated in vacuo and purification by preparative HPLC (C18 120 g, 20-80% MeCN in water (10 mM ammonium bicarbonate)) gave the title compound (11 mg, 74%). LCMS (Method 3): 4.79 min, 1065.7 [M+H]⁺. ¹H NMR (400 MHz, DMSO-d₆) δ 8.65 (t, 1H), 8.07 (t, 1H), 7.52-7.43 (m, 4H), 7.37 (d, 1H), 7.29-7.20 (m, 1H), 7.15 (d, 1H), 5.79-5.68 (m, 1H), 5.17-5.05 (m, 2H), 4.52 (t, 1H), 4.34 (t, 2H), 4.01-3.86 (m, 2H), 3.81 (dd, 1H), 3.66-3.45 (m, 4H), 3.43-3.22 (m, 3H), 3.07-2.93 (m, 2H), 2.85-2.65 (m, 3H), 2.62 (s, 3H), 2.43-2.35 (m, 7H), 2.33 (s, 3H), 2.20 (d, 1H), 2.09 (d, 1H), 1.69-1.58 (m, 4H), 1.57-1.43 (m, 3H), 1.42-1.28 (m, 2H), 1.28-1.12 (m, 1H), 1.05 (s, 3H), 0.61-0.36 (m, 4H).

### Example 3: (1S,2R)-2-((S)-5-Chloro-8-((5-((2-(2-(2-((S)-4-(4-chlorophenyl)-2,3,9-trimethyl-6H-thieno[3,2-f][1,2,4]triazolo[4,3-a][1,4]diazepin-6-yl)acetamido)acetamido)ethoxy)methyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid

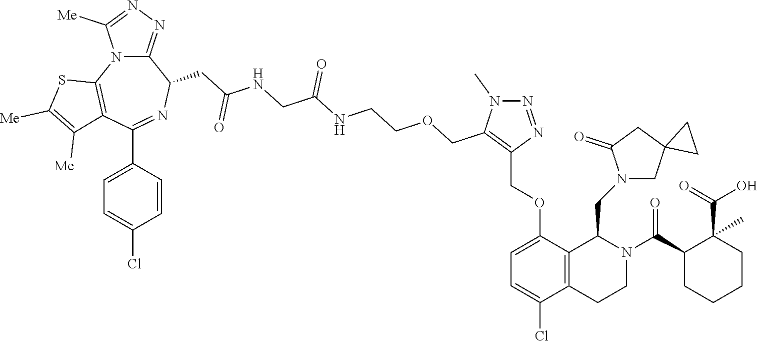 [SMILES: Cc1sc2c(c1C)C(c1ccc(Cl)cc1)=N[C@@H](CC(=O)NCC(=O)NCCOCc1c(COc3ccc(Cl)c4c3[C@@H](CN3CC5(CC5)CC3=O)N(C(=O)[C@@H]3CCCC[C@]3(C)C(=O)O)CC4)nnn1C)c1nnc(C)n1-2]

To a stirred solution of Intermediate 21 (97 mg, 0.08 mmol) and triethylsilane (0.2 mL, 1.3 mmol, CAS: 617-86-7) in DCM (0.8 mL) was added TFA (0.05 mL, 0.6 mmol) and the reaction mixture stirred at rt for 4 h. The mixture was concentrated in vacuo, and purified by preparative HPLC (C18, 5-60% MeCN in water (10 mM ammonium bicarbonate)). The residue was taken up in MeCN/water (1:1) and freeze dried to provide the title compound (41 mg, 45%). LCMS (Method 3): 4.94 min, 1082.7 [M+H]⁺. ¹H NMR (400 MHz, DMSO-d₆) δ 12.14-11.59 (bm, 1H), 8.69-8.49 (bm, 1H), 8.01-7.79 (bm, 1H), 7.51-7.41 (m, 4H), 7.33 (d, 1H), 7.13 (d, 1H), 5.79-5.73 (m, 1H), 5.21 (s, 2H), 4.76-4.65 (m, 2H), 4.52 (t, 1H), 4.05-3.88 (m, 5H), 3.79 (dd, 1H), 3.68-3.53 (m, 2H), 3.53-3.45 (m, 2H), 3.41-3.22 (m, 5H), 3.05-2.93 (m, 2H), 2.86-2.76 (m, 1H), 2.76-2.62 (m, 2H), 2.58 (s, 3H), 2.41 (s, 3H), 2.35-2.22 (m, 1H), 2.18 (d, 1H), 2.10 (d, 1H), 1.72-1.47 (m, 6H), 1.46-1.17 (m, 4H), 1.11 (s, 3H), 0.62-0.37 (m, 4H).

### Example 4: (1S,2R)-2-((S)-5-Chloro-8-((5-((2-(3-(2-((S)-4-(4-chlorophenyl)-2,3,9-trimethyl-6H-thieno[3,2-f][1,2,4]triazolo[4,3-a][1,4]diazepin-6-yl)acetamido)propanamido)ethoxy)methyl)-1-methyl-1H-1,2,3-triazol-4-yl)methoxy)-1-((6-oxo-5-azaspiro[2.4]heptan-5-yl)methyl)-1,2,3,4-tetrahydroisoguinoline-2-carbonyl)-1-methylcyclohexane-1-carboxylic acid

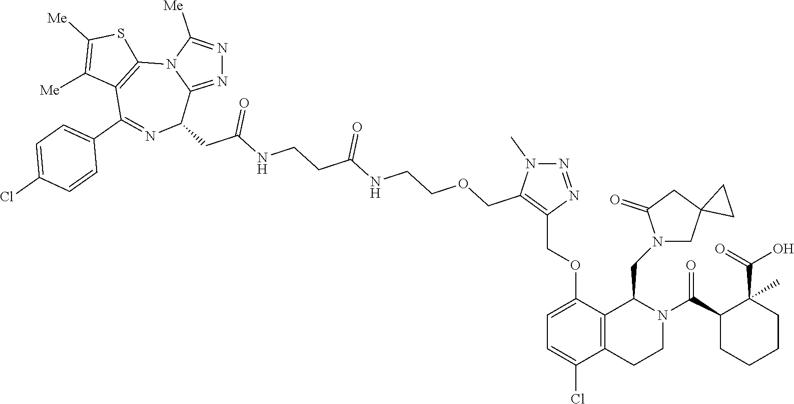 [SMILES: Cc1sc2c(c1C)C(c1ccc(Cl)cc1)=N[C@@H](CC(=O)NCCC(=O)NCCOCc1c(COc3ccc(Cl)c4c3[C@@H](CN3CC5(CC5)CC3=O)N(C(=O)[C@@H]3CCCC[C@]3(C)C(=O)O)CC4)nnn1C)c1nnc(C)n1-2]

To a stirred solution of Intermediate 22 (69 mg, 0.06 mmol) and triethylsilane (0.14 mL, 0.89 mmol) in DCM (0.8 mL) was added TFA (0.03 mL, 0.44 mmol) and the reaction mixture stirred at rt for 4 h. The mixture was concentrated in vacuo, purified by preparative HPLC (C18, 5-60% MeCN in water (10 mM ammonium bicarbonate)). The residue was taken up in MeCN/water (1:1) and freeze dried to provide the title compound (30 mg, 48%). LCMS (Method 3): 4.85 min, 1096.7 [M+H]⁺. ¹H NMR (400 MHz, DMSO-d₆) δ 11.95-11.75 (bm, 1H), 8.35-8.13 (bm, 1H), 8.08-7.86 (bm, 1H), 7.51-7.46 (m, 2H), 7.45-7.39 (m, 2H), 7.34 (d, 1H), 7.13 (d, 1H), 5.79-5.73 (m, 1H), 5.25-5.16 (m, 2H), 4.76-4.64 (m, 2H), 4.50 (t, 1H), 4.05-3.87 (m, 5H), 3.64-3.52 (m, 1H), 3.50-3.41 (m, 2H), 3.39-3.13 (m, 7H), 3.03-2.93 (m, 2H), 2.85-2.77 (m, 1H), 2.77-2.61 (m, 2H), 2.59 (s, 3H), 2.41 (s, 3H), 2.35-2.23 (m, 3H), 2.21-2.06 (m, 2H), 1.70-1.47 (m, 6H), 1.45-1.18 (m, 4H), 1.10 (s, 3H), 0.61-0.40 (m, 4H).

### Example 5: (1S,2R)-2-((S)-5-Chloro-8-((5-(14-((S)-4-(4-chlorophenyl)-2,3,9-trimethyl-6H-thieno[3,2-f][1,2,4]triazolo[4,3-a][1,4]diazepin-6-yl)-6,13-dioxo-2,9-dioxa-5,12-diazatetradecyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid

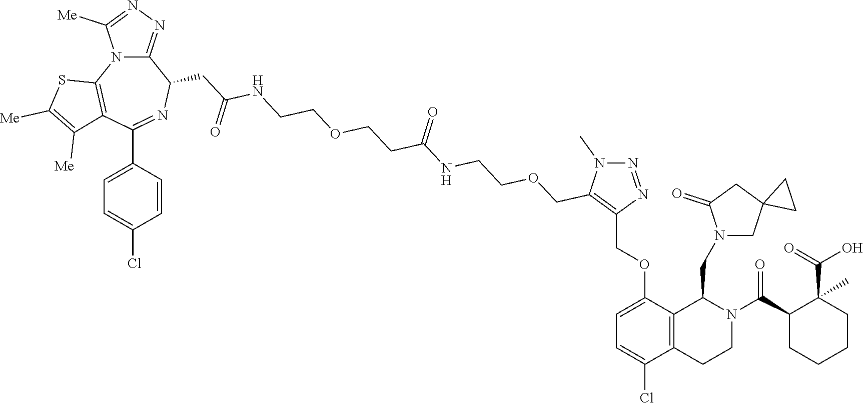 [SMILES: Cc1sc2c(c1C)C(c1ccc(Cl)cc1)=N[C@@H](CC(=O)NCCOCCC(=O)NCCOCc1c(COc3ccc(Cl)c4c3[C@@H](CN3CC5(CC5)CC3=O)N(C(=O)[C@@H]3CCCC[C@]3(C)C(=O)O)CC4)nnn1C)c1nnc(C)n1-2]

To a stirred solution of Intermediate 23 (38 mg, 0.03 mmol) and triethylsilane (0.08 mL, 0.47 mmol) in DCM (0.5 mL) was added TFA (0.02 mL, 0.24 mmol) and the reaction mixture stirred at rt for 4 h. The mixture was concentrated in vacuo, purified by preparative HPLC (C18, 20-80% MeCN in water (10 mM ammonium bicarbonate). The residue was taken up in MeCN/water (1:1) and freeze dried to provide the title compound (13 mg, 36%). LCMS (Method 4): 4.30 min, 1140.7 [M+H]⁺. ¹H NMR (400 MHz, DMSO-d₆) δ 8.33-8.22 (bm, 1H), 7.51-7.46 (m, 2H), 7.45-7.39 (m, 2H), 7.34 (d, 1H), 7.13 (d, 1H), 5.78-5.72 (m, 1H), 5.24-5.16 (m, 2H), 4.75-4.61 (m, 2H), 4.53-4.48 (m, 1H), 4.04-3.85 (m, 5H), 3.65-3.52 (m, 3H), 3.49-3.12 (m, 11H), 3.03-2.93 (m, 2H), 2.85-2.62 (m, 3H), 2.59 (s, 3H), 2.41 (s, 3H), 2.36-2.25 (m, 3H), 2.18 (d, 1H), 2.10 (d, 1H), 1.69-1.45 (m, 6H), 1.44-1.17 (m, 4H), 1.09 (s, 3H), 0.60-0.40 (m, 4H).

### Example 6: (1S,2R)-2-((S)-5-Chloro-8-(((S)-1-(2-(2-(2-(2-((S)-4-(4-chlorophenyl)-2,3,9-trimethyl-6H-thieno[3,2-f][1,2,4]triazolo[4,3-a][1,4]diazepin-6-yl)acetamido)acetamido)ethoxy)acetyl)pyrrolidin-3-yl)oxy)-1-((6-oxo-5-azaspiro[2.4]heptan-5-yl)methyl)-1,2,3,4-tetrahydroisoquinoline-2-carbonyl)-1-methylcyclohexane-1-carboxylic acid

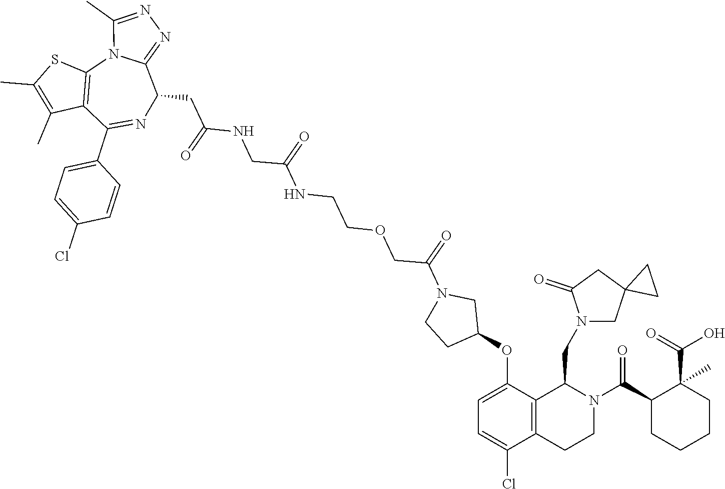

To a stirred solution of Intermediate 24 (27 mg, 0.022 mmol) and triethylsilane (0.06 mL, 0.35 mmol) in DCM (0.2 mL) was added TFA (0.01 mL, 0.17 mmol) and the reaction mixture stirred at rt for 4 h. The mixture was concentrated in vacuo, purified by preparative HPLC (C18, 5-60% MeCN in water (10 mM ammonium bicarbonate)). The residue was taken up in MeCN/water (1:1) and freeze dried to provide the title compound (4.4 mg, 12%). LCMS (Method 3): 4.83 min, 1084.5 [M+H]⁺. ¹H NMR (400 MHz, CDCl₃) -complex mixture including characteristic peaks for the title compound.

### COMPARATIVE EXAMPLES

### Comparative Example 1: (1S,2R)-2-((S)-5-Chloro-8-((1,5-dimethyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-N-(2-(2-(2-((S)-4-(4-chlorophenyl)-2,3,9-trimethyl-6H-thieno[3,2-f][1,2,4]triazolo[4,3-a][1,4]diazepin-6-yl)acetamido)acetamido)ethyl)-1-methylcyclohexane-1-carboxamide

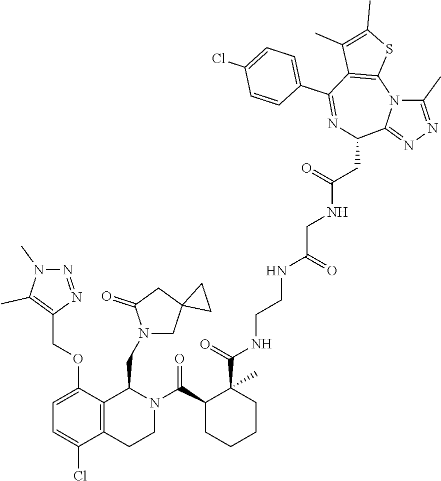

### Comparative Example 2: (1S,2R)-2-((S)-5-chloro-8-((1,5-dimethyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-N-(2-(2-(2-((S)-4-(4-chlorophenyl)-2,3,9-trimethyl-6H-thieno[3,2-f][1,2,4]triazolo[4,3-a][1,4]diazepin-6-yl)acetamido)ethoxy)ethyl)-1-methylcyclohexane-1-carboxamide

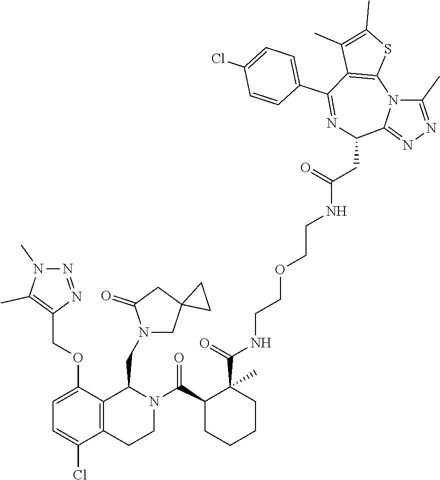

### Comparative Example 3: (1S,2R)-2-((S)-5-Chloro-8-((1,5-dimethyl-1H-1,2,3-triazol-4-yl)methoxy)-1-((6-oxo-5-azaspiro[2.4]heptan-5-yl)methyl)-1,2,3,4-tetrahydroisoguinoline-2-carbonyl)-N-(2-(2-(2-(2-((S)-4-(4-chlorophenyl)-2,3,9-trimethyl-6H-thieno[3,2-f][1,2,4]triazolo[4,3-a][1,4]diazepin-6-yl)acetamido)ethoxy)ethoxy)ethyl)-1-methylcyclohexane-1-carboxamide

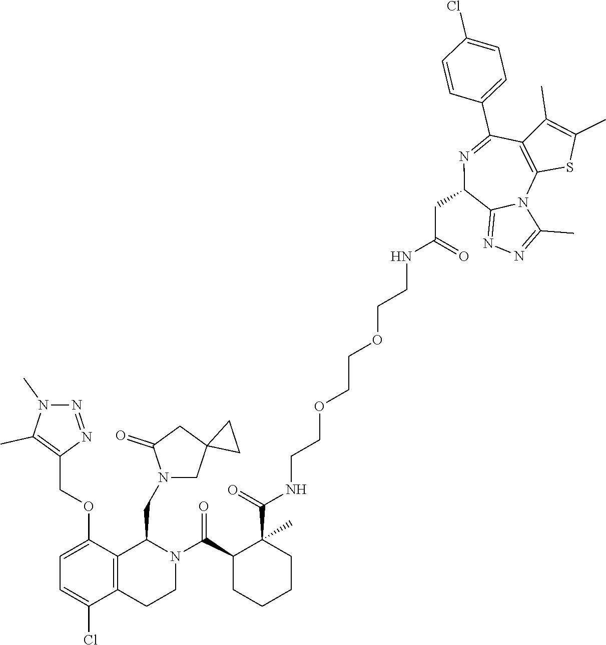

### Biological Assays

Beas2B NQO1 mRNA Cell Based Assay

The up-regulation of the NRF2 mediated gene NAD(P)H:quinone acceptor oxidoreductase 1 (NQO1) was measured using the following assay method: BEAS-2B cells (ATCC CRL-9609) were plated in 96-well clear plates at 20,000 cells/well in 75 μL of cell culture media and incubated overnight (37° C., 5% CO₂). On day 2, 25 μL of compound or controls were added to the cells for 24 h. On day 3, the medium was aspirated from the plate and the Cells-to-CT™ 1-Step TaqMan® Kit (Ambion A25603) was used to perform expression analysis directly from cultured cells without RNA purification according to the manufacturer's instructions.

Briefly, cells were washed with ice cold PBS and 22.5 μL of room-temperature DNase/Lysis solution was added to the cells and incubated at room temperature for 5 minutes. To stop the reaction, 2.25 μL of stop solution was added to the cell lysate. The samples were diluted 1:5 using nuclease free water and 2.5 μL transferred into the PCR plate. Real-time PCR was performed using the C-1000 Thermal Cycler (Bio-Rad) using human beta actin as the internal control. The cDNA was amplified with a specific primer for NQO1 using the 1-step RT-PCR master mix (Ambion Cells-to-CT™ 1-Step TaqMan® Kit A25603). The primers/probes sets that were used for amplification of cDNA were obtained from TaqMan Gene Expression Assays (Applied Biosystems). The comparative CT (ΔΔCT) relative quantification method was used to calculate the relative mRNA level of the target gene NQO1 as described in the Applied Biosystems Chemistry Guide. The data are expressed as an increase in target gene mRNA compared to vehicle (0.1% DMSO) control and the EC₅₀ values were determined by fitting the data to a four parameter logistic fit using XLfit or XE Runner within ActivityBase. EC₅₀ values for the tested compounds are shown in Table 1.

TABLE 1

| Compound | EC₅₀ (nM) |
|---|---|
| Example 1 | 187 |
| Example 2 | 458 |
| Example 3 | 280 |
| Example 4 | 660 |
| Example 5 | 337 |
| Example 6 | 2486 |
| Comparative Example 1 | 212 |
| Comparative Example 2 | 75 |
| Comparative Example 3 | 68 |

### BRD4 Degradation Assay Protocol Summary

MDA-MB-468 cells were cultured routinely in DMEM supplemented with 10% heat inactivated FBS and 1% Penicillin-Streptomycin (Thermofisher Scientific) at 37° C. 5% CO₂. Cells were seeded in 6-well plates at 4*10\hat{ }5 cells per well and left to adhere overnight. Example compounds were solubilised in DMSO to make 10 mM stocks followed by serial dilutions in DMSO to 200× final concentration. These were further diluted in culture media to give 20× treatment stocks before addition to cells to a 1× final concentration. Cells were then incubated at 37° C. 5% CO₂ for 24 or 48 hours after which cells were lysed in RIPA buffer supplemented with cOmplete Mini Protease Inhibitor Cocktail and PhosSTOP™ (Roche). Lysates were clarified by centrifugation at 13,000 RPM for 10 minutes and the protein concentration of the resulting supernatants determined by BCA assay (ThermoFisher Scientific) as per manufacturer's instructions. Lysates were denatured and reduced in NuPAGE LDS sample buffer and sample reducing agent before 10 μg was loaded onto NuPAGE 2-8% Tris-Acetate Mini Protein Gels (ThermoFisher Scientific). Protein was transferred to nitrocellulose membranes using a Trans-Blot Turbo Transfer System (Bio-Rad) before blocking for 1 hour in Intercept (TBS) Blocking Buffer (Li-Cor). Membranes were then incubated overnight with BRD4 (ab128874; Abcam) and α-tubulin (T6074; Sigma-Aldrich) antibodies diluted 1:2000 and 1:16,000 respectively in Tris-buffered saline 0.05% Tween 20 (TBS-T). The following day, membranes were washed with TBS-T before addition of anti-rabbit IRDye 800 CW and anti-mouse IRDye 680LT secondary antibodies (LiCor) (diluted 1:10,000 in TBS-T) for one hour. Membranes were then washed with TBS-T before imaging using a LICOR Odyssey Infrared Imaging System with Image Studio Ver 5.2 software (LiCor). Densitometry analysis was carried out in the LiCor image studio software where BRD4 signal was normalised to α-tubulin signal. Percentage degradation of BRD4 relative to the DMSO treated negative control was calculated as: =100−(signal of interest/signal of control sample*100). BRD4 degradation by dBET1 was used as positive control for each plate. Data obtained for the percentage degradation of BRD4 upon treatment with the Example compounds 1-6 and Comparative Example compounds 1-3 at 24 hour incubation are shown in Table 2 and with 48 hour incubation are shown in Table 3.

The Beas2B assay data in Table 1 shows that all the compounds tested (Examples 1-6 and Comparative Examples 1-3) were capable of Keap1 binding and Nrf2 activation, as measured by an increase in downstream NQO1 mRNA. The BRD4 degradation data in Tables 2 and 3 show that (i) Example 1 gave almost complete target protein degradation at ≥0.1 μM and gave up to 75% degradation even at 0.01 μM; (ii) Examples 1-6 all provided dose-responsive degradation of BRD4, with greater than 60% degradation of the long isoform, and greater than 77% degradation of the short isoform, after 48 hours incubation; and (iii) Comparative Examples 1-3 did not significantly degrade either BRD isoform or demonstrate dose-responsive degradation of the target protein.

TABLE 2

| | | % Mean Degradation of BRD4 (n = 2), 24 h | | | | | | | | | |
|---|---|---|---|---|---|---|---|---|---|---|---|
| | | | | | | | | Comp. | Comp. | Comp. | BRD4 |
| | | Ex. 1 | Ex. 2 | Ex. 3 | Ex. 4 | Ex. 5 | Ex. 6 | Ex. 1 | Ex. 2 | Ex. 3 | isoform |
| Conc | 0.01 | 75 | | | | | | | | | Long |
| (μM) | 0.1 | 99 | 77 | 23 | −37 | 1 | 9 | 11 | −33 | 34 | |
| | 1 | 101 | 93 | 80 | 26 | 65 | 52 | 18 | 3 | 45 | |
| | 5 | | 91 | 75 | 43 | 69 | 95 | 5 | 14 | 49 | |
| Conc | 0.01 | 69 | | | | | | | | | Short |
| (μM) | 0.1 | 97 | 84 | 31 | 18 | −5 | 21 | 18 | −6 | 5 | |
| | 1 | 96 | 95 | 87 | 65 | 76 | 60 | 42 | 9 | 31 | |
| | 5 | | 97 | 87 | 75 | 78 | 95 | 43 | 26 | 18 | |

TABLE 3

| | | % Mean Degradation of BRD4 (n = 2), 48 h | | | | | | | | | |
|---|---|---|---|---|---|---|---|---|---|---|---|
| | | | | | | | | Comp. | Comp. | Comp. | BRD4 |
| | | Ex. 1 | Ex. 2 | Ex. 3 | Ex. 4 | Ex. 5 | Ex. 6 | Ex. 1 | Ex. 2 | Ex. 3 | isoform |
| Conc | 0.01 | 74 | | | | | | | | | Long |
| (μM) | 0.1 | 97 | 78 | 14 | −19 | 34 | −27 | −39 | −26 | 13 | |
| | 1 | 99 | 93 | 67 | 56 | 78 | 43 | −27 | −61 | 30 | |
| | 5 | | 94 | 60 | 71 | 82 | 82 | −47 | −124 | 20 | |
| Conc | 0.01 | 68 | | | | | | | | | Short |
| (μM) | 0.1 | 93 | 71 | 25 | −2 | 40 | 1 | −11 | −15 | 23 | |
| | 1 | 100 | 92 | 77 | 65 | 78 | 44 | 24 | 13 | 32 | |
| | 5 | | 93 | 79 | 77 | 80 | 81 | 28 | 20 | −4 | |

1. A compound of Formula I, or a pharmaceutically acceptable salt thereof:
KBG-L-PBG (I)
wherein L is a bivalent linker group covalently linking the KBG to the PBG;
PBG is a Protein Binding Group comprising a moiety having a binding affinity to a target protein of interest; and
KBG is a KEAP1 Binding Group having the structure shown below: 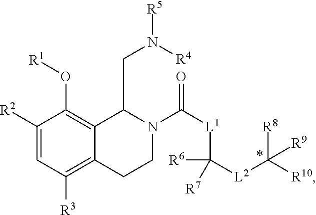
wherein:
R¹ is selected from C₁₋₄alkylene-R¹¹, heterocyclyl and 8-10 membered bicyclic heteroaryl, wherein said heterocyclyl is optionally substituted with one or more substituents independently selected from C₁₋₄alkyl, —C(O)—R¹², SO₂—R¹³, C₁₋₃alkylene-OR¹⁴ and heteroaryl which is optionally substituted with one or more substituents independently selected from C₁₋₄alkyl, C₃₋₇cycloalkyl, halo, OH, C₁₋₃alkoxy and cyano; and wherein said 8-10 membered bicyclic heteroaryl is optionally substituted with one or more substituents independently selected from C₁₋₄alkyl, C₃₋₇cycloalkyl, halo, OH and C₁₋₃alkoxy;
R² is selected from hydrogen, fluoro, chloro and C₁₋₃alkyl;
R³ is selected from hydrogen, fluoro, chloro, bromo, C₁₋₃alkoxy, C₁₋₃alkyl, C₁₋₃haloalkyl and cyano;
R⁴ is hydrogen or C₁₋₄alkyl;
R⁵ is —C(O)—C₁₋₄alkyl, —C(O)-heteroaryl or —C(O)-aryl, wherein said heteroaryl and aryl are optionally substituted with one or more substituents selected from C₁₋₄alkyl, halo, hydroxy, C₁₋₃alkoxy, CO₂R¹⁵ and cyano; or
R⁴ and R⁵, taken together with the nitrogen atom to which they are attached, form a 4-, 5-, or 6-membered heterocyclyl ring, wherein said heterocyclyl ring:
comprises one or more —C(O)— moieties attached to the nitrogen atom;
optionally is fused to an aryl or heteroaryl ring;
optionally is spiro-attached to a C₃₋₇cycloalkyl group; and
optionally is substituted with one or more substituents independently selected from C₁₋₄alkyl, halo, OH, C₁₋₃alkoxy, C₁₋₃haloalkyl, cyano, NR¹⁶R¹⁷, C(O)R¹⁸, S(O)R¹⁹ and SO₂R²⁰;
L¹ and L² are independently selected from a bond and —CR²¹R²²—;
R⁶ and R⁷ are independently selected from hydrogen, C₁₋₄alkyl, and C₃₋₇cycloalkyl; or
R⁶ and R⁷, taken together with the carbon atom to which they are attached, form a 3- , 4-, 5-, or 6-membered cycloalkyl ring;
R⁸ is selected from C(═O)NR^{8a}R^{8b}, —C(═O)Rao, CO₂R²³, C(O)NHSO₂C₁₋₃alkyl, tetrazolyl, 3-trifluoromethyl-1,2,4-triazol-5-yl and a carboxylic acid mimetic group selected from hydroxamic acids, hydroxamic esters, phosphonic acids, phosphinic acids, sulfonic acids, sulfinic acids, sulphonamides, sulfonyl ureas, acyl ureas, thiazolidine dione, oxazolidine dione, oxadiazol-5(4H)-one, thiadiazol-5(4H)-one, oxathiadiazole-2-oxide, oxadiazol-5(4H)-thione, isoxazole, tetramic acid, cyclopentane-1,3-diones and cyclopentane-1,2-diones;
R^{8a} is hydrogen or C₁₋₆alkyl;
R^{8b} is hydrogen, C₁₋₆alkyl or C₃₋₇cycloalkyl, wherein the C₁₋₆alkyl or C₃₋₇cycloalkyl groups are optionally substituted with one or more substituents independently selected from C₁₋₄alkyl, C₃₋₇cycloalkyl, halo, OH, C₁₋₃alkoxy, C₁₋₃haloalkyl, cyano, NR¹⁶R¹⁷, C(O)R¹⁸, S(O)R¹⁹ and SO₂R²⁰; or
R^{8a} and R^{8b}, taken together with the nitrogen atom to which they are attached, form a 3-, 4-, 5-, 6-, or 7-membered heterocyclyl ring, wherein the heterocyclyl ring optionally contains one or more additional heteroatoms selected from oxygen, nitrogen and sulfur, and is optionally substituted with one or more substituents independently selected from C₁₋₄alkyl, C₃₋₇cycloalkyl, halo, OH, C₁₋₃alkoxy, C₁₋₃haloalkyl, cyano, NR¹⁶R¹⁷, C(O)R¹⁸, S(O)R¹⁹ and SO₂R²⁰;
R^{8c} is C₁₋₃alkyl or C₁₋₃haloalkyl;
R⁹ is selected from hydrogen, C₁₋₄alkyl, hydroxy, C₁₋₃alkoxy and halo;
R¹⁰ is selected from hydrogen and C₁₋₄alkyl; or
R⁹ and R¹⁰, taken together with the carbon atom to which they are attached, form a 3-, 4-, 5-, or 6-membered cycloalkyl ring; or
L² is a bond and R⁷ and R¹⁰, taken together with the atoms to which they are attached, form a 4-, 5-, 6- or 7-membered cycloalkyl or heterocyclyl ring, wherein:
said heterocyclyl ring contains 1 or 2 heteroatoms independently selected from nitrogen, oxygen and sulfur;
said cycloalkyl ring optionally comprises 1 or 2 carbon-carbon double bonds and is optionally bridged by a C₁₋₃alkylene group connecting two carbon atoms of the ring, or R⁹ is optionally a C₁₋₃alkylene group connecting C* to a carbon atom of the ring; and
said cycloalkyl and heterocyclyl rings are optionally substituted with one or more substituents independently selected from C₁₋₄alkyl, halo, OH, C₁₋₃alkoxy, C₁₋₃haloalkyl and deuterium;
R¹¹ is selected from —C(O)—R²⁴, —SO₂—R²⁵, —NR²⁶C(O)—R²⁷, —NR²⁸SO₂—R²⁹, heterocyclyl, aryl and heteroaryl, wherein said heterocyclyl, aryl and heteroaryl groups are optionally substituted with one or more substituents independently selected from C₁₋₄alkyl, C₁₋₃haloalkyl, C₃₋₇cycloalkyl, C₁₋₄alkylene-R³⁰, halo, OH, C₁₋₃alkoxy, heterocyclyl and cyano; and said heterocyclyl group is optionally substituted with one or more substituents independently selected from C₁₋₄alkyl, C₁₋₃haloalkyl, C₃₋₇cycloalkyl, C₁₋₄alkylene-R³⁰, halo, OH, C₁₋₃alkoxy, oxo and cyano;
R¹² is selected from C₁₋₄alkyl, C₃₋₇cycloalkyl, OR³¹, NR³²R³³, aryl and heteroaryl, wherein said aryl and heteroaryl are optionally substituted with one or more substituents independently selected from C₁₋₄alkyl, halo, OH, C₁₋₃alkoxy and cyano;
R¹³ is selected from C₁₋₄alkyl, C₃₋₇cycloalkyl, heteroaryl, heterocyclyl and NR³⁴R³⁵, wherein said heteroaryl and heterocyclyl are optionally substituted with one or more substituents independently selected from C₁₋₄alkyl, halo, OH, C₁₋₃alkoxy and cyano;
R¹⁷ is selected from hydrogen, C₁₋₄alkyl, C(O)C₁₋₃alkyl and C(O)NR³⁶R³⁷;
R¹⁸, R¹⁹ and R²⁰ are independently selected from C₁₋₄alkyl, OH, C₁₋₃alkoxy and NR³⁸R³⁹;
R²³ is selected from hydrogen and C₁₋₄alkyl;
R²⁴ is selected from C₁₋₄alkyl, NR⁴⁰R⁴¹ and OR⁴²;
R²⁵ is selected from C₁₋₄alkyl and NR⁴³R⁴⁴;
R²⁷ is selected from C₁₋₄alkyl, C₃₋₇cycloalkyl, C₁₋₃haloalkyl, heterocyclyl, aryl and heteroaryl, wherein said aryl and heteroaryl are optionally substituted with one or more substituents independently selected from C₁₋₄alkyl, C₁₋₃haloalkyl, C₃₋₇cycloalkyl, C₁₋₄alkylene-R⁴⁵, halo, OH, C₁₋₃alkoxy and cyano;
R²⁹ is selected from C₁₋₄alkyl, C₃₋₇cycloalkyl, C₁₋₃haloalkyl, aryl and heteroaryl, wherein said aryl and heteroaryl are optionally substituted with one or more substituents independently selected from C₁₋₄alkyl, C₁₋₃haloalkyl, C₃₋₇cycloalkyl, C₁₋₄alkylene-R⁴⁶ halo, OH, C₁₋₃alkoxy and cyano;
R³⁰ is selected from hydroxy, C₁₋₃alkoxy, C₃₋₇cycloalkyl, cyano and NR⁴⁷R⁴⁸;
R⁴⁰ is selected from hydrogen and C₁₋₄alkyl;
R⁴¹ is selected from hydrogen, C₁₋₄alkyl, C₃₋₇cycloalkyl, C₁₋₃alkoxy, aryl and heteroaryl; or
R⁴⁰ and R⁴¹, taken together with the nitrogen atom to which they are attached, form a 4-, 5-, or 6-membered heteroaryl or heterocyclyl ring, wherein said heteroaryl and heterocyclyl rings are optionally substituted with one or more substituents independently selected from C₁₋₄alkyl, halo, OH, C₁₋₃alkoxy, C₃₋₇cycloalkyl and cyano;
R⁴⁵ and R⁴⁶ are independently selected from hydroxy, C₁₋₃alkoxy and C₃₋₇cycloalkyl; and
R¹⁴, R¹⁵, R¹⁶, R²¹, R²², R²⁶, R²⁸, R³¹, R³², R³³, R³⁴, R³⁵, R³⁶, R³⁷, R³⁸, R³⁹, R⁴², R⁴³, R⁴⁴, R⁴⁷ and R⁴⁸ are independently selected from hydrogen, C₁₋₄alkyl and C₃₋₇cycloalkyl.

2. The compound according to claim 1, wherein the KBG has one of the structural formulae (II) to (VIII) shown below: 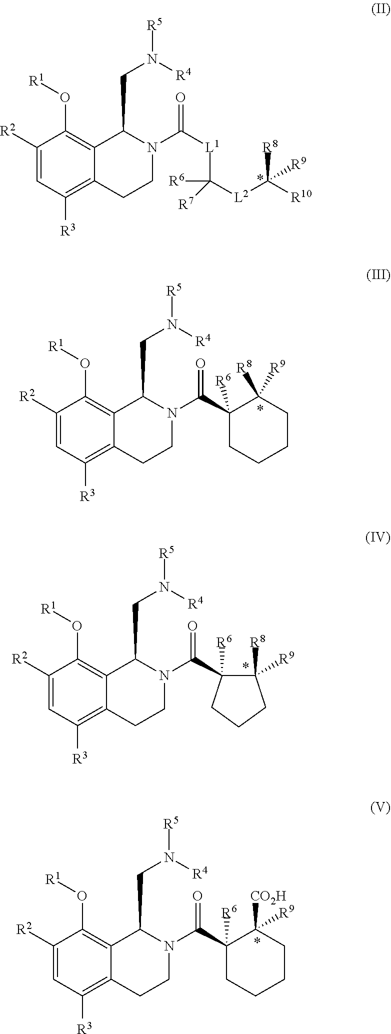 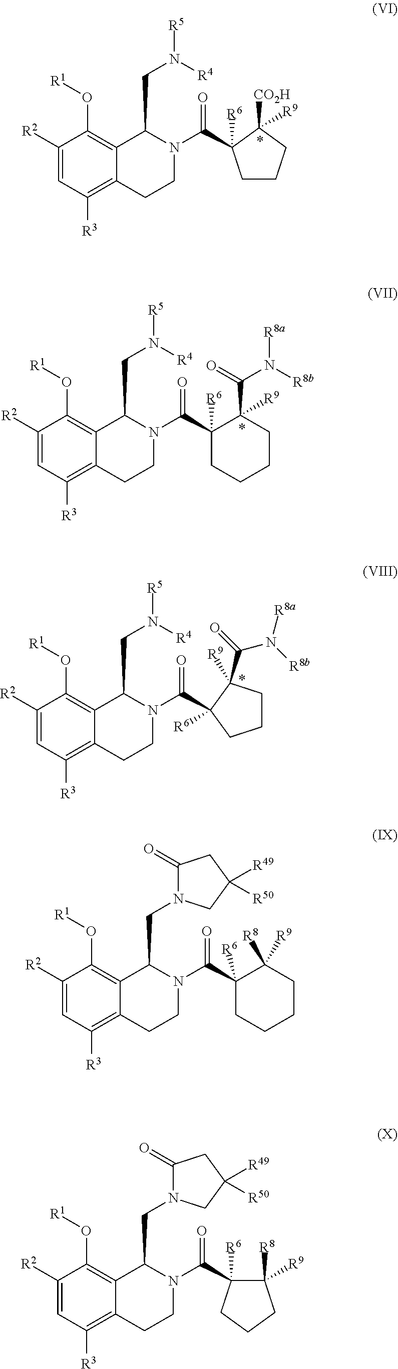
wherein R¹ to R¹⁰, L¹ and L² are as defined in claim 1; the cyclohexyl or cyclopentyl ring in formulae (Ill) to (VIII) is optionally substituted with one or more substituents independently selected from C₁₋₄alkyl, halo, OH, C₁₋₃alkoxy and deuterium, and is optionally bridged by a C₁₋₃alkylene group connecting two carbon atoms of the ring, and R⁹ is optionally a C₁₋₃alkylene group connecting C* to a carbon atom of the ring.

3. The compound according to any one of claims 1 to 2, wherein R⁸ is CO₂R²³ and R²³ is hydrogen.

4. The compound according to any one of claims 1 to 3, wherein R⁹ is hydrogen or C₁₋₄alkyl.

5. The compound according to claim 4, wherein R⁹ is methyl.

6. The compound according to any one of claims 1 to 5, wherein R¹ is C₁₋₄alkylene-R¹¹.

7. The compound according to claim 6, wherein R¹ is CH₂—R¹¹.

8. The compound according to any one of claims 1 to 7, wherein R¹¹ is:
(A) selected from —C(O)—R²⁴, —NR²⁶C(O)—R²⁷, and heteroaryl, wherein said heteroaryl is optionally substituted with one or more substituents independently selected from C₁₋₄alkyl, C₁₋₃haloalkyl, C₃₋₇cycloalkyl, C₁₋₄alkylene-R³⁰, halo, OH, C₁₋₃alkoxy, heterocyclyl and cyano;
(B) heteroaryl optionally substituted with one or more substituents independently selected from C₁₋₄alkyl, C₁₋₃haloalkyl, C₃₋₇cycloalkyl, C₁₋₄alkylene-R³⁰, halo, OH, C₁₋₃alkoxy, heterocyclyl and cyano;
(C) heteroaryl optionally substituted with one or more substituents independently selected from methyl, ethyl, isopropyl, difluoromethyl, trifluoromethyl, chloro, fluoro, cyclopropyl, methoxy, cyano, oxetanyl, CH₂—R³⁰ and C₂H₄—R³⁰; OR
(D) pyridyl, pyrimidinyl, pyridazinyl, oxazolyl, isoxazolyl, 1,2,3-triazolyl, 1,2,4-oxadiazolyl, benzotriazolyl, benzisoxazolyl, isoxazolopyridinyl, imidazopyridinyl or triazolopyridinyl, each optionally substituted with one or more substituents independently selected from C₁₋₄alkyl, C₁₋₃haloalkyl, C₃₋₇cycloalkyl, C₁₋₄alkylene-R³⁰, halo, OH, C₁₋₃alkoxy, heterocyclyl and cyano.

9. The compound according to any one of claims 1 to 7, wherein R¹¹ is selected from: 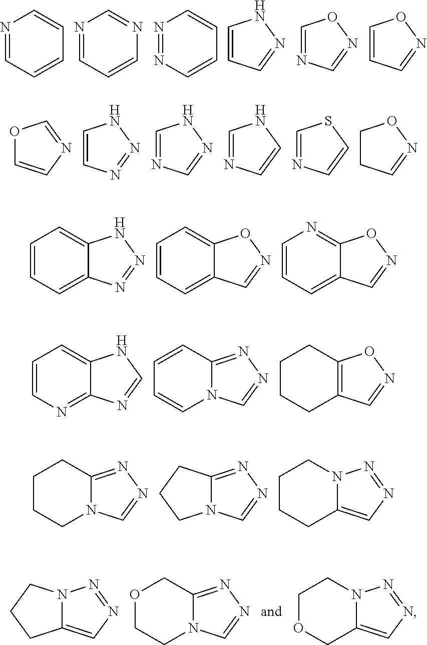
optionally substituted with one or more substituents independently selected from C₁₋₄alkyl, C₁₋₃haloalkyl, C₃₋₇cycloalkyl, C₁₋₄alkylene-R³⁰, halo, OH, C₁₋₃alkoxy, heterocyclyl and cyano.

10. The compound according to any one of claims 1 to 5, wherein R¹ is selected from one of the following groups: 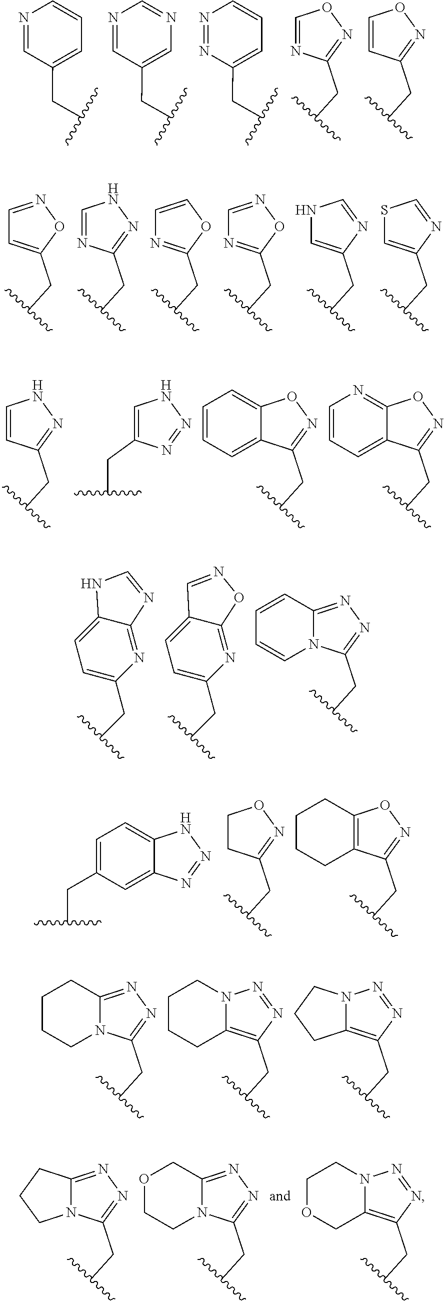
wherein [symbol] represents the point of attachment of the group to the oxygen atom of the rest of the compound and wherein each cyclic group is optionally substituted with one or more substituents independently selected from C₁₋₄alkyl, C₁₋₃haloalkyl, C₃₋₇cycloalkyl, C₁₋₄alkylene-R³⁰, halo, OH, C₁₋₃alkoxy, heterocyclyl and cyano.

11. The compound according to any one of claims 1 to 5, wherein R¹ is:
(A) heterocyclyl optionally substituted with one or more substituents independently selected from —C(O)—R¹², SO₂—R¹³, heteroaryl and C₁₋₃alkylene-OR¹⁴, wherein said heteroaryl is optionally substituted with one or more substituents independently selected from C₁₋₄alkyl, C₃₋₇cycloalkyl, halo, OH, C₁₋₃alkoxy and cyano;
(B) piperidinyl or pyrrolidinyl, each optionally substituted with one or more substituents independently selected from —C(O)—R¹², SO₂—R¹³, heteroaryl and C₁₋₃alkylene-OR¹⁴, wherein said heteroaryl is optionally substituted with one or more substituents independently selected from C₁₋₄alkyl, C₃₋₇cycloalkyl, halo, OH, C₁₋₃alkoxy and cyano;
(C) pyrrolidinyl optionally substituted with one or more substituents independently selected from —C(O)—R¹², SO₂—R¹³, heteroaryl and C₁₋₃alkylene-OR¹⁴, wherein said heteroaryl is optionally substituted with C₁₋₄alkyl or C₃₋₇cycloalkyl; OR
(D) selected from one of the following groups: 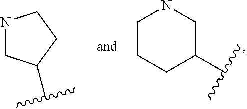
wherein [symbol] represents the point of attachment of the group to the oxygen atom of the rest of the compound and wherein each group is optionally substituted with one or more substituents independently selected from —C(O)—R¹², SO₂—R¹³, heteroaryl and C₁₋₃alkylene-OR¹⁴, wherein said heteroaryl is optionally substituted with C₁₋₄alkyl or C₃₋₇cycloalkyl.

12. The compound according to any one of claims 1 to 11, wherein R² is hydrogen or fluoro.

13. The compound according to any one of claims 1 to 12, wherein R³ is hydrogen or chloro.

14. The compound according to any one of claims 1 to 13, wherein R⁴ is hydrogen and R⁵ is —C(O)—C₁₋₄alkyl or —C(O)-aryl, wherein said aryl is optionally substituted with one or more substituents selected from C₁₋₄alkyl, halo, hydroxy, C₁₋₃alkoxy, CO₂R¹⁵ and cyano.

15. The compound according to any one of claims 1 to 13, wherein R⁴ and R⁵, taken together with the nitrogen atom to which they are attached, form:
(A) a 5- or 6-membered heterocyclyl ring, wherein said heterocyclyl ring comprises one or more —C(O)— moieties attached to the nitrogen atom and is optionally fused to an aryl ring, or optionally spiro-attached to a C₃₋₇cycloalkyl group, and is optionally substituted with one or more substituents independently selected from C₁₋₄alkyl, halo, OH, C₁₋₃alkoxy, C₁₋₃haloalkyl, cyano, NR¹⁶R¹⁷, C(O)R¹⁸, S(O)R¹⁹ and SO₂R²⁰;
(B) a 5- or 6-membered heterocyclyl ring, wherein said heterocyclyl ring comprises a —C(O)— moiety attached to the nitrogen atom and is optionally fused to an aryl ring, or optionally spiro-attached to a C₃₋₇cycloalkyl group, and wherein said heterocyclyl ring is optionally substituted with one or more substituents independently selected from C₁₋₄alkyl, halo and OH; OR
(C) a heterocyclic moiety selected from one of the following: 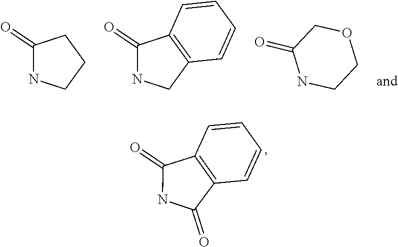
wherein the saturated ring of the heterocyclic moiety is optionally spiro-attached to a C₃₋₇cycloalkyl group, and wherein said heterocyclic moiety is optionally substituted with one or more substituents independently selected from C₁₋₄alkyl, halo and OH.

16. The compound according to any one of claims 1 to 15, wherein R^{8a} is hydrogen.

17. The compound according to any one of claims 1 to 16, wherein R^{8b} is:
a) hydrogen, C₁₋₄alkyl or C₃₋₅cycloalkyl, wherein the C₁₋₄alkyl group is optionally substituted with one or more substituents independently selected from halo, OH, C₁₋₃alkoxy, C₃₋₇cycloalkyl and cyano;
b) C₁₋₄alkyl or C₃₋₅cycloalkyl, wherein the C₁₋₄alkyl group is optionally substituted with one or more substituents independently selected from fluoro, OH, C₁₋₃alkoxy, C₃₋₇cycloalkyl and cyano; OR c) methyl.

18. The compound according to claim 1, wherein the KBG is selected from one of the following groups:
(1S,2R)-2-((S)-1-((1,3-dioxoisoindolin-2-yl)methyl)-8-(2-(5-methylisoxazole-3-carboxamido)ethoxy)-1,2,3,4-tetrahydroisoquinoline-2-carbonyl)cyclohexane-1-carboxylic acid;
(1S,2R)-2-((S)-8-(2-(Benzo[d]oxazole-2-carboxamido)ethoxy)-1-((1,3-dioxoisoindolin-2-yl)methyl)-1,2,3,4-tetrahydroisoquinoline-2-carbonyl)cyclohexane-1-carboxylic acid;
(1S,2R)-2-((S)-8-(((S)-1-Acetylpyrrolidin-3-yl)oxy)-1-((1,3-dioxoisoindolin-2-yl)methyl)-1,2,3,4-tetrahydroisoquinoline-2-carbonyl)cyclohexane-1-carboxylic acid;
(1S,2R)-2-((S)-1-((1,3-dioxoisoindolin-2-yl)methyl)-8-(((S)-1-(5-methylisoxazole-3-carbonyl)pyrrolidin-3-yl)oxy)-1,2,3,4-tetrahydroisoquinoline-2-carbonyl)cyclohexane-1-carboxylic acid;
(1S,2R)-2-((S)-5-bromo-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)cyclohexane-1-carboxylic acid;
(1S,2R)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)cyclohexane-1-carboxylic acid;
(1S,2R)-2-((S)-1-((1,3-dioxoisoindolin-2-yl)methyl)-8-(((S)-1-(2-methylthiazole-5-carbonyl)pyrrolidin-3-yl)oxy)-1,2,3,4-tetrahydroisoquinoline-2-carbonyl)cyclohexane-1-carboxylic acid;
(1S,2R)-2-((S)-1-((1,3-dioxoisoindolin-2-yl)methyl)-8-((1-methyl-1H-benzo[d][1,2,3]triazol-5-yl)methoxy)-1,2,3,4-tetrahydroisoquinoline-2-carbonyl)cyclohexane-1-carboxylic acid;
(1S,2R)-2-((S)-1-((1,3-dioxoisoindolin-2-yl)methyl)-8-((1-methyl-1H-1,2,3-triazol-4-yl)methoxy)-1,2,3,4-tetrahydroisoquinoline-2-carbonyl)cyclohexane-1-carboxylic acid;
(1S,2R)-2-((S)-1-((1,3-dioxoisoindolin-2-yl)methyl)-8-((1-methyl-1H-benzo[d]imidazol-5-yl)methoxy)-1,2,3,4-tetrahydroisoquinoline-2-carbonyl)cyclohexane-1-carboxylic acid;
(1S,2R)-2-((S)-8-(((S)-1-acetylpyrrolidin-3-yl)oxy)-5-bromo-1-((1,3-dioxoisoindolin-2-yl)methyl)-1,2,3,4-tetrahydroisoquinoline-2-carbonyl)cyclohexane-1-carboxylic acid;
(1S,2R)-2-((S)-8-(((S)-1-acetylpyrrolidin-3-yl)oxy)-5-chloro-1-((1,3-dioxoisoindolin-2-yl)methyl)-1,2,3,4-tetrahydroisoquinoline-2-carbonyl)cyclohexane-1-carboxylic acid;
(1S,2R)-2-((S)-8-(((S)-1-acetylpyrrolidin-3-yl)oxy)-5,7-dichloro-1-((1,3-dioxoisoindolin-2-yl)methyl)-1,2,3,4-tetrahydroisoquinoline-2-carbonyl)cyclohexane-1-carboxylic acid;
(1S,2R)-2-((S)-5-chloro-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)cyclohexane-1-carboxylic acid;
(1S,2R)-2-((S)-1-((1-oxoisoindolin-2-yl)methyl)-8-(((S)-1-(thiazole-5-carbonyl)pyrrolidin-3-yl)oxy)-1,2,3,4-tetrahydroisoquinoline-2-carbonyl)cyclohexane-1-carboxylic acid;
(1S,2R)-2-((S)-8-([1,2,4]triazolo[4,3-a]pyridin-3-ylmethoxy)-5-bromo-1-((1-oxoisoindolin-2-yl)methyl)-1,2,3,4-tetrahydroisoquinoline-2-carbonyl)cyclohexane-1-carboxylic acid;
(1S,2R)-2-((S)-8-((1-isopropyl-1H-1,2,3-triazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-8-((1-methyl-1H-benzo[d][1,2,3]triazol-5-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-8-(((S)-1-acetylpyrrolidin-3-yl)oxy)-5-bromo-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-(((S)-1-(2-methylthiazole-5-carbonyl)pyrrolidin-3-yl)oxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-(((S)-1-(5-methylthiazol-2-yl)pyrrolidin-3-yl)oxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-(((S)-1-(5-methylpyridazin-3-yl)pyrrolidin-3-yl)oxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((4-methyl-4H-1,2,4-triazol-3-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((5-methyl-1,2,4-oxadiazol-3-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-Bromo-8-((1-methyl-1H-imidazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((5-methylisoxazol-3-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((2-ethyl-2H-1,2,3-triazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((1S)-5-bromo-8-((5-methylthiazol-2-yl)methoxy)-1-((1-oxo-1,3,3a,4,5,6-hexahydro-2H-isoindol-2-yl)methyl)-1,2,3,4-tetrahydroisoquinoline-2-carbonyl)cyclohexane-1-carboxylic acid;
(1S,2R)-2-((S)-5-bromo-8-((1-methyl-1H-1,2,4-triazol-5-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-1-((1-oxoisoindolin-2-yl)methyl)-8-(pyridazin-3-ylmethoxy)-1,2,3,4-tetrahydroisoquinoline-2-carbonyl)cyclohexane-1-carboxylic acid;
(1S,2R)-2-((S)-5-bromo-8-((1-(cyclopropylmethyl)-1H-1,2,3-triazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-(imidazo[1,2-a]pyridin-7-ylmethoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((1-methyl-1H-benzo[d]imidazol-5-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((1-methyl-1H-1,2,3-triazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((1-isopropyl-1H-1,2,3-triazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((5-methyl-1,3,4-oxadiazol-2-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((7-methoxy-1-methyl-1H-benzo[d][1,2,3]triazol-5-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((1-methyl-1H-benzo[d][1,2,3]triazol-5-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((1-methyl-1H-1,2,4-triazol-3-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((1-isopropyl-1H-imidazol-2-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((5-(difluoromethyl)-1-methyl-1H-1,2,3-triazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((4-ethyl-4H-1,2,4-triazol-3-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-8-((5-cyano-1-ethyl-1H-imidazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-bromo-1-((1-oxoisoindolin-2-yl)methyl)-8-((5,6,7,8-tetrahydro-[1,2,4]triazolo[4,3-a]pyridin-3-yl)methoxy)-1,2,3,4-tetrahydroisoquinoline-2-carbonyl)cyclohexane-1-carboxylic acid;
(1S,2R)-2-((1S)-5-bromo-8-(1-(1-methyl-1H-1,2,3-triazol-4-yl)ethoxy)-1-((1-oxoisoindolin-2-yl)methyl)-1,2,3,4-tetrahydroisoquinoline-2-carbonyl)cyclohexane-1-carboxylic acid;
(1S,2R)-2-((1S)-5-bromo-8-(1-(1-isopropyl-1H-1,2,3-triazol-4-yl)ethoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-chloro-8-((1-methyl-1H-benzo[d][1,2,3]triazol-5-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-chloro-8-((5-methylisoxazol-3-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-8-([1,2,4]triazolo[4,3-a]pyridin-3-ylmethoxy)-5-chloro-1-((1-oxoisoindolin-2-yl)methyl)-1,2,3,4-tetrahydroisoquinoline-2-carbonyl)cyclohexane-1-carboxylic acid;
(1S,2R)-2-((S)-5-chloro-1-((1-oxoisoindolin-2-yl)methyl)-8-((1-(2,2,2-trifluoroethyl)-1H-imidazol-2-yl)methoxy)-1,2,3,4-tetrahydroisoquinoline-2-carbonyl)cyclohexane-1-carboxylic acid;
(1S,2R)-2-((S)-5-chloro-8-((1-(2,2-difluoroethyl)-1H-imidazol-2-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-chloro-8-((5-(difluoromethyl)-1-methyl-1H-1,2,3-triazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-chloro-8-((1-methyl-5-(trifluoromethyl)-1H-1,2,3-triazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-chloro-8-((1-ethyl-1H-pyrazol-3-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-chloro-8-((5-(difluoromethyl)-1-methyl-1H-imidazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-chloro-8-((1-isopropyl-1H-1,2,3-triazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-5-fluoro-8-((1-isopropyl-1H-1,2,3-triazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-8-((1-isopropyl-1H-1,2,3-triazol-4-yl)methoxy)-5-methyl-1-((1-oxoisoindolin-2-yl)methyl)-1,2,3,4-tetrahydroisoquinoline-2-carbonyl)cyclohexane-1-carboxylic acid;
(1S,2R)-2-((S)-5-cyano-8-((1-isopropyl-1H-1,2,3-triazol-4-yl)methoxy)-1-((1-oxoisoindolin-2-yl)methyl)-1,2,3,4-tetrahydroisoquinoline-2-carbonyl)cyclohexane-1-carboxylic acid;
(1S,2R)-2-((S)-8-((1-isopropyl-1H-1,2,3-triazol-4-yl)methoxy)-5-methoxy-1-((1-oxoisoindolin-2-yl)methyl)-1,2,3,4-tetrahydroisoquinoline-2-carbonyl)cyclohexane-1-carboxylic acid;
(1S,2R)-2-((S)-8-((1-isopropyl-1H-1,2,3-triazol-4-yl)methoxy)-1-((1-oxoisoindolin-2-yl)methyl)-5-(trifluoromethyl)-1,2,3,4-tetrahydroisoquinoline-2-carbonyl)cyclohexane-1-carboxylic acid;
(1S,2R)-2-((S)-5-bromo-1-((2-oxopyrrolidin-1-yl)methyl)-8-(((S)-1-(thiazole-5-carbonyl)pyrrolidin-3-yl)oxy)-1,2,3,4-tetrahydroisoquinoline-2-carbonyl)cyclohexane-1-carboxylic acid;
(1S,2R)-2-((S)-8-([1,2,4]triazolo[4,3-a]pyridin-3-ylmethoxy)-5-bromo-1-((2-oxopyrrolidin-1-yl)methyl)-1,2,3,4-tetrahydroisoquinoline-2-carbonyl)cyclohexane-1-carboxylic acid;
(1S,2R)-2-((S)-5-bromo-8-((5-methylthiazol-2-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-5-bromo-8-((5-isopropyl-1,2,4-oxadiazol-3-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-8-(benzo[d]isoxazol-3-ylmethoxy)-5-bromo-1-((2-oxopyrrolidin-1-yl)methyl)-1,2,3,4-tetrahydroisoquinoline-2-carbonyl)cyclohexane-1-carboxylic acid;
(1S,2R)-2-((S)-8-(benzo[d]isothiazol-3-ylmethoxy)-5-chloro-1-((2-oxopyrrolidin-1-yl)methyl)-1,2,3,4-tetrahydroisoquinoline-2-carbonyl)cyclohexane-1-carboxylic acid;
(1S,2R)-2-((S)-5-chloro-1-((2-oxopyrrolidin-1-yl)methyl)-8-(pyridin-2-ylmethoxy)-1,2,3,4-tetrahydroisoquinoline-2-carbonyl)cyclohexane-1-carboxylic acid;
(1S,2R)-2-((S)-5-chloro-8-((5-methylisothiazol-3-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-5-chloro-8-(isothiazol-3-ylmethoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-8-(benzo[d]isoxazol-3-ylmethoxy)-5-chloro-1-((2-oxopyrrolidin-1-yl)methyl)-1,2,3,4-tetrahydroisoquinoline-2-carbonyl)cyclohexane-1-carboxylic acid;
(1S,2R)-2-((1S)-5-bromo-8-((1-isopropyl-1H-1,2,3-triazol-4-yl)methoxy)-1-((3-methyl-2-oxopyrrolidin-1-yl)methyl)-1,2,3,4-tetrahydroisoquinoline-2-carbonyl)cyclohexane-1-carboxylic acid;
(1S,2R)-2-((S)-5-bromo-8-((1-isopropyl-1H-1,2,3-triazol-4-yl)methoxy)-1-(((R)-4-methyl-2-oxopyrrolidin-1-yl)methyl)-1,2,3,4-tetrahydroisoquinoline-2-carbonyl)cyclohexane-1-carboxylic acid;
(1S,2R)-2-((S)-5-bromo-8-((1-isopropyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)cyclohexane-1-carboxylic acid;
(1S,2R)-2-((S)-5-bromo-8-((1-isopropyl-1H-1,2,3-triazol-4-yl)methoxy)-1-(((S)-4-methyl-2-oxopyrrolidin-1-yl)methyl)-1,2,3,4-tetrahydroisoquinoline-2-carbonyl)cyclohexane-1-carboxylic acid;
(1S,2R)-2-((S)-5-chloro-1-((2-oxopyrrolidin-1-yl)methyl)-8-(pyridin-3-ylmethoxy)-1,2,3,4-tetrahydroisoquinoline-2-carbonyl)cyclohexane-1-carboxylic acid;
(1S,2R)-2-((S)-5-chloro-8-((5-(difluoromethyl)-1-ethyl-1H-1,2,3-triazol-4-yl)methoxy)-1-((2-oxopyrrolidin-1-yl)methyl)-1,2,3,4-tetrahydroisoquinoline-2-carbonyl)cyclohexane-1-carboxylic acid;
(R)-4-((S)-8-(benzo[d]isoxazol-3-ylmethoxy)-5-chloro-1-((2-oxopyrrolidin-1-yl)methyl)-3,4-dihydroisoquinolin-2(1H)-yl)-3-methyl-4-oxobutanoic acid;
1-(((S)-2-((1R,2S)-2-(2H-tetrazol-5-yl)cyclohexane-1-carbonyl)-8-(benzo[d]isoxazol-3-ylmethoxy)-5-chloro-1,2,3,4-tetrahydroisoquinolin-1-yl)methyl)pyrrolidin-2-one;
(1S,2R)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-5-chloro-8-((5-isopropyl-1,2,4-oxadiazol-3-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-5-chloro-8-((5-(difluoromethyl)-1-methyl-1H-1,2,3-triazol-4-yl)methoxy)-1-((4-methyl-1H-pyrazol-1-yl)methyl)-1,2,3,4-tetrahydroisoquinoline-2-carbonyl)cyclohexane-1-carboxylic acid;
(1S,2R)-2-((S)-5-chloro-1-((2-oxopyrrolidin-1-yl)methyl)-8-(pyrimidin-5-ylmethoxy)-1,2,3,4-tetrahydroisoquinoline-2-carbonyl)cyclohexane-1-carboxylic acid;
(1S,2R)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)cyclopentane-1-carboxylic acid;
(1R,2S)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)cyclopentane-1-carboxylic acid;
(1S,2R)-2-((S)-5-chloro-1-((2-oxopyrrolidin-1-yl)methyl)-8-(pyridazin-3-ylmethoxy)-1,2,3,4-tetrahydroisoquinoline-2-carbonyl)cyclohexane-1-carboxylic acid;
(1S,2R)-2-((S)-5-chloro-8-((3-methyl-3H-imidazo[4,5-b]pyridin-6-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-5-chloro-8-((1-methyl-1H-benzo[d][1,2,3]triazol-5-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-(imidazo[1,2-a]pyrimidin-2-ylmethoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-5-chloro-8-(isoxazolo[5,4-b]pyridin-3-ylmethoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-8-([1,2,4]triazolo[4,3-a]pyridin-3-ylmethoxy)-5-chloro-1-((2-oxopyrrolidin-1-yl)methyl)-1,2,3,4-tetrahydroisoquinoline-2-carbonyl)cyclohexane-1-carboxylic acid;
(1S,2R)-2-((S)-5-chloro-8-((3-methylisoxazolo[5,4-b]pyridin-6-yl)methoxy)-1-((2-oxopyrrolidin-1-yl)methyl)-1,2,3,4-tetrahydroisoquinoline-2-carbonyl)cyclohexane-1-carboxylic acid;
3-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)bicyclo[2.2.1]heptane-2-carboxylic acid;
3-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)tetrahydrofuran-2-carboxylic acid;
(1S,2R)-2-((S)-5-chloro-8-((1-methyl-1H-indazol-3-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-5-chloro-8-((3-methyl-3H-imidazo[4,5-b]pyridin-5-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-8-(benzo[d]isoxazol-3-ylmethoxy)-5-chloro-1-((2-oxopyrrolidin-1-yl)methyl)-1,2,3,4-tetrahydroisoquinoline-2-carbonyl)-1-methylcyclohexane-1-carboxylic acid;
2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-4,4-difluorocyclopentane-1-carboxylic acid;
(1S,2R)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-1-methylcyclopentane-1-carboxylic acid;
(1S,2R)-2-((S)-5-chloro-8-((4,5-dimethyl-4H-1,2,4-triazol-3-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-5-chloro-8-((1-methyl-1H-1,2,3-triazol-4-yl)methoxy)-1-((2-oxopyrrolidin-1-yl)methyl)-1,2,3,4-tetrahydroisoquinoline-2-carbonyl)cyclohexane-1-carboxylic acid;
(1R,2S)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-1-fluorocyclohexane-1-carboxylic acid;
(1S,2R)-2-((S)-5-chloro-8-((1-methyl-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((1,5-dimethyl-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((1-methyl-5-(trifluoromethyl)-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-methyl-1H-1,2,3-triazol-4-yl)methoxy)-1-(((R)-4-methyl-2-oxopyrrolidin-1-yl)methyl)-1,2,3,4-tetrahydroisoquinoline-2-carbonyl)-1-methylcyclopentane-1-carboxylic acid;
(1S,2R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclopentane-1-carboxylic acid;
(1S,2R)-2-((S)-5-chloro-8-((5-(difluoromethyl)-1-methyl-1H-1,2,3-triazol-4-yl)methoxy)-1-(((R)-4-methyl-2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-methyl-1H-1,2,3-triazol-4-yl)methoxy)-1-(((R)-3-methyl-2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-methyl-1H-1,2,3-triazol-4-yl)methoxy)-1-(((R)-3-methyl-2-oxopyrrolidin-1-yl)methyl)-1,2,3,4-tetrahydroisoquinoline-2-carbonyl)-1-methylcyclopentane-1-carboxylic acid;
(1S,2R)-2-((S)-5-chloro-8-((5-(difluoromethyl)-1-methyl-1H-1,2,3-triazol-4-yl)methoxy)-1-(((S)-3-methyl-2-oxopyrrolidin-1-yl)methyl)-1,2,3,4-tetrahydroisoquinoline-2-carbonyl)-1-methylcyclopentane-1-carboxylic acid;
(1S,2R)-2-((S)-5-chloro-8-((5-(difluoromethyl)-1-methyl-1H-1,2,3-triazol-4-yl)methoxy)-1-(((S)-3-methyl-2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-methyl-1H-1,2,3-triazol-4-yl)methoxy)-1-(((S)-4-methyl-2-oxopyrrolidin-1-yl)methyl)-1,2,3,4-tetrahydroisoquinoline-2-carbonyl)-1-methylcyclopentane-1-carboxylic acid;
(1S,2R)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-1-ethylcyclohexane-1-carboxylic acid;
(1S,2R)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-1-ethylcyclopentane-1-carboxylic acid;
(1S,2R)-2-((S)-5-chloro-8-((5-(difluoromethyl)-1-ethyl-1H-1,2,3-triazol-4-yl)methoxy)-1-((2-oxopyrrolidin-1-yl)methyl)-1,2,3,4-tetrahydroisoquinoline-2-carbonyl)-1-methylcyclopentane-1-carboxylic acid;
(1S,2R)-2-((S)-5-chloro-8-((5-(difluoromethyl)-1-ethyl-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-isopropyl-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-isopropyl-1H-1,2,3-triazol-4-yl)methoxy)-1-((2-oxopyrrolidin-1-yl)methyl)-1,2,3,4-tetrahydroisoquinoline-2-carbonyl)-1-methylcyclopentane-1-carboxylic acid;
(1S,2R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((4,5-dimethylisoxazol-3-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((4-chloro-5-methylisoxazol-3-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((4-methylisoxazol-5-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-(methoxymethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-(2-methoxyethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-cyclopropyl-1-methyl-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-1-(((S)-3-methyl-2-oxopyrrolidin-1-yl)methyl)-8-((1-methyl-5-(trifluoromethyl)-1H-1,2,3-triazol-4-yl)methoxy)-1,2,3,4-tetrahydroisoquinoline-2-carbonyl)-1-methylcyclohexane-1-carboxylic acid;
(1S,2R)-2-((S)-5-chloro-1-(((R)-3-methyl-2-oxopyrrolidin-1-yl)methyl)-8-((1-methyl-5-(trifluoromethyl)-1H-1,2,3-triazol-4-yl)methoxy)-1,2,3,4-tetrahydroisoquinoline-2-carbonyl)-1-methylcyclohexane-1-carboxylic acid;
(1S,2R)-2-((S)-5-chloro-1-(((R)-4-methyl-2-oxopyrrolidin-1-yl)methyl)-8-((1-methyl-5-(trifluoromethyl)-1H-1,2,3-triazol-4-yl)methoxy)-1,2,3,4-tetrahydroisoquinoline-2-carbonyl)-1-methylcyclohexane-1-carboxylic acid;
(1S,2S)-2-((S)-5-chloro-1-(((R)-4-methyl-2-oxopyrrolidin-1-yl)methyl)-8-((1-methyl-5-(trifluoromethyl)-1H-1,2,3-triazol-4-yl)methoxy)-1,2,3,4-tetrahydroisoquinoline-2-carbonyl)-1-methylcyclohexane-1-carboxylic acid;
(1S,2R)-2-((S)-5-chloro-8-((1-cyclopropyl-5-(difluoromethyl)-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((4-(difluoromethyl) pyrimidin-5-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5,5-dimethyl-4,5-dihydroisoxazol-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((4-methylisoxazol-3-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-1-((2-oxopyrrolidin-1-yl)methyl)-8-((4,5,6,7-tetrahydrobenzo[d]isoxazol-3-yl)methoxy)-1,2,3,4-tetrahydroisoquinoline-2-carbonyl)-1-methylcyclohexane-1-carboxylic acid;
(1S,2R)-2-((S)-8-((2,5-bis(difluoromethyl)-2H-1,2,3-triazol-4-yl)methoxy)-5-chloro-1-((6-oxo-5-azaspiro[2.4]heptan-5-yl)methyl)-1,2,3,4-tetrahydroisoquinoline-2-carbonyl)-1-methylcyclohexane-1-carboxylic acid;
2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)tetrahydro-2H-pyran-3-carboxylic acid;
(R)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)bicyclo[2.2.2]octane-1-carboxylic acid;
(1S,2R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic-4,4-d2 acid;
(1S,2R)-2-((S)-5-chloro-8-((1,5-dimethyl-1H-1,2,3-triazol-4-yl)methoxy)-7-fluoro-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-7-fluoro-1-((6-oxo-5-azaspiro[2.4]heptan-5-yl)methyl)-8-((4,5,6,7-tetrahydro-[1,2,3]triazolo[1,5-a]pyridin-3-yl)methoxy)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-(2-methoxyethyl)-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5,6-dihydro-4H-pyrrolo[1,2-c][1,2,3]triazol-3-yl)methoxy)-7-fluoro-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-methyl-1H-1,2,3-triazol-4-yl)methoxy)-7-fluoro-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((1-methyl-2-oxo-1,2-dihydropyridin-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2S)-2-((S)-5-chloro-8-((5-(difluoromethyl)-1-methyl-1H-1,2,3-triazol-4-yl)methoxy)-1-(((R)-4-methyl-2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((1-methyl-5-(trifluoromethyl)-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
1-(((S)-5-chloro-8-((5-(difluoromethyl)-1-methyl-1H-1,2,3-triazol-4-yl)methoxy)-2-((1R,2S)-2-methyl-2-(2H-tetrazol-5-yl)cyclohexane-1-carbonyl)-1,2,3,4-tetrahydroisoquinolin-1-yl)methyl)pyrrolidin-2-one;
(1S,2R)-2-((S)-5-chloro-8-((5-(difluoromethyl)-1-methyl-1H-1,2,3-triazol-4-yl)methoxy)-1-((4,4-dimethyl-2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((1-methyl-5-(trifluoromethyl)-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclopentane-1-carboxylic acid;
(1S,2R)-2-((S)-5-chloro-8-((5-methyl-4-(trifluoromethyl)isoxazol-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-(2,2,2-trifluoroethyl)-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((1,5-dimethyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((4-(difluoromethyl)-5-methylisoxazol-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5-cyclopropyl-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5-(methoxymethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((1-methyl-1H-1,2,3-triazol-4-yl)methoxy)-1-(((R)-4-methyl-2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-(methoxymethyl)-1-methyl-1H-1,2,3-triazol-4-yl)methoxy)-1-(((R)-4-methyl-2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-cyclopropyl-1-methyl-1H-1,2,3-triazol-4-yl)methoxy)-1-(((R)-4-methyl-2-oxopyrrolidin-1-yl)methyl)-1,2,3,4-tetrahydroisoquinoline-2-carbonyl)-1-methylcyclohexane-1-carboxylic acid;
(1S,2R)-2-((1S)-5-chloro-8-((7-fluoro-2,7a-dihydrobenzo[d]isoxazol-3-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((6,7-difluorobenzo[d]isoxazol-3-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-methylisoxazol-3-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((3-methyl-1,2,4-oxadiazol-5-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-methyl-1,2,4-oxadiazol-3-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2S)-2-((S)-5-chloro-8-((1-methyl-5-(trifluoromethyl)-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-1-((6-oxo-5-azaspiro[2.4]heptan-5-yl)methyl)-8-((4,5,6,7-tetrahydro-[1,2,3]triazolo[1,5-a]pyridin-3-yl)methoxy)-1,2,3,4-tetrahydroisoquinoline-2-carbonyl)-1-methylcyclohexane-1-carboxylic acid;
(1S,2R)-2-((S)-5-chloro-1-((6-oxo-5-azaspiro[2.4]heptan-5-yl)methyl)-8-((4-(trifluoromethyl) pyrimidin-5-yl)methoxy)-1,2,3,4-tetrahydroisoquinoline-2-carbonyl)-1-methylcyclohexane-1-carboxylic acid;
(1S,2R)-2-((S)-5-chloro-8-((1-cyclopropyl-5-(difluoromethyl)-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((1-(2-(dimethylamino)ethyl)-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-1-methyl-2-((S)-5-methyl-8-((1-methyl-5-(trifluoromethyl)-1H-1,2,3-triazol-4-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-5-chloro-8-((4-methyl-4H-1,2,4-triazol-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5,6-dihydro-8H-[1,2,4]triazolo[3,4-c][1,4]oxazin-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((4-cyclopropyl-5-methyl-4H-1,2,4-triazol-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-fluoro-8-((1-methyl-5-(trifluoromethyl)-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((4,5-dimethyl-4H-1,2,4-triazol-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-1-((6-oxo-5-azaspiro[2.4]heptan-5-yl)methyl)-8-((4,5,6,7-tetrahydro-[1,2,3]triazolo[1,5-a]pyridin-3-yl)methoxy)-1,2,3,4-tetrahydroisoquinoline-2-carbonyl)-1-methylcyclopentane-1-carboxylic acid;
(1S,2R)-2-((S)-5-chloro-8-((5,6-dihydro-4H-pyrrolo[1,2-c][1,2,3]triazol-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-fluoro-1-((6-oxo-5-azaspiro[2.4]heptan-5-yl)methyl)-8-((4,5,6,7-tetrahydro-[1,2,3]triazolo[1,5-a]pyridin-3-yl)methoxy)-1,2,3,4-tetrahydroisoquinoline-2-carbonyl)-1-methylcyclohexane-1-carboxylic acid;
(1S,2R)-2-((S)-5-chloro-8-((6,7-dihydro-5H-pyrrolo[2,1-c][1,2,4]triazol-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5-cyclopropyl-4-methyl-4H-1,2,4-triazol-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((1-methyl-1H-imidazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-1-((6-oxo-5-azaspiro[2.4]heptan-5-yl)methyl)-8-((5,6,7,8-tetrahydro-[1,2,4]triazolo[4,3-a]pyridin-3-yl)methoxy)-1,2,3,4-tetrahydroisoquinoline-2-carbonyl)-1-methylcyclohexane-1-carboxylic acid;
(1S,2R)-2-((S)-5-chloro-8-(((S)-1-(methylsulfonyl)pyrrolidin-3-yl)oxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-8-(((S)-1-acetylpyrrolidin-3-yl)oxy)-5-chloro-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((6,7-dihydro-4H-[1,2,3]triazolo[5,1-c][1,4]oxazin-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-1-methyl-2-((S)-8-((1-methyl-5-(trifluoromethyl)-1H-1,2,3-triazol-4-yl)methoxy)-1-((2-oxopyrrolidin-1-yl)methyl)-1,2,3,4-tetrahydroisoquinoline-2-carbonyl)cyclohexane-1-carboxylic acid;
(1S,2R)-2-((S)-5-bromo-8-((1-methyl-5-(trifluoromethyl)-1H-1,2,3-triazol-4-yl)methoxy)-1-((2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-(oxetan-3-yl)-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-8-((1,5-bis(difluoromethyl)-1H-1,2,3-triazol-4-yl)methoxy)-5-chloro-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-methyl-1H-1,2,3-triazol-4-yl)methoxy)-1-(((R)-2-methyl-5-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-methyl-1H-1,2,3-triazol-4-yl)methoxy)-1-(((S)-2-methyl-5-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-1-((3-oxomorpholino)methyl)-8-((4,5,6,7-tetrahydro-[1,2,3]triazolo[1,5-a]pyridin-3-yl)methoxy)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)cyclohexane-1-carboxylic acid;
(1S,2R)-2-((S)-5-bromo-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((1-methyl-5-(oxetan-3-yl)-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-methyl-1H-1,2,3-triazol-4-yl)methoxy)-1-((3-oxomorpholino)methyl)-1,2,3,4-tetrahydroisoquinoline-2-carbonyl)-1-methylcyclohexane-1-carboxylic acid;
(1S,6R)-6-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohex-3-ene-1-carboxylic acid;
5-(((S)-2-((1R,2S)-2-(1H-tetrazol-5-yl)cyclohexane-1-carbonyl)-5-chloro-8-((5-(difluoromethyl)-1-methyl-1H-1,2,3-triazol-4-yl)methoxy)-1,2,3,4-tetrahydroisoquinolin-1-yl)methyl)-5-azaspiro[2.4]heptan-6-one;
(1S,2R)-2-((S)-5-chloro-8-((5-(difluoromethyl)-1-methyl-1H-1,2,3-triazol-4-yl)methoxy)-7-fluoro-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)thiazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5-methylthiazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((2-methyl-2H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1R,3S,4R,6S)-4-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-3-methylbicyclo[4.1.0]heptane-3-carboxylic acid;
(1S,3S,4R,6R)-4-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-3-methylbicyclo[4.1.0]heptane-3-carboxylic acid;
(1S,2R)-2-((1S)-5-chloro-8-((6,7-dihydro-5H-pyrrolo[2,1-c][1,2,4]triazol-7-yl)oxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R,4S,5R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic-4,5-d2 acid;
(1S,2R)-2-((S)-5-chloro-8-((1-methyl-2-oxo-1,2-dihydropyridin-3-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((1S)-5-chloro-8-((5-(difluoromethyl)-1-methyl-1H-1,2,3-triazol-4-yl)methoxy)-1-((2-oxo-4-(trifluoromethyl) pyrrolidin-1-yl)methyl)-1,2,3,4-tetrahydroisoquinoline-2-carbonyl)-1-methylcyclohexane-1-carboxylic acid;
(1S,2R)-2-((S)-5-chloro-8-((1-methyl-6-oxo-1,6-dihydropyridin-2-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((1S)-5-chloro-8-((1-methyl-1,4,5,6-tetrahydrocyclopenta[d][1,2,3]triazol-5-yl)oxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-4,4-difluoro-1-methylcyclohexane-1-carboxylic acid;
(1S,2R,4S)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-4-fluoro-1-methylcyclohexane-1-carboxylic acid;
(1S,2R,4R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic-4-d acid;
(1S,2R,4S)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-4-hydroxy-1-methylcyclohexane-1-carboxylic acid;
(1S,2R,5S)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-5-hydroxy-1-methylcyclohexane-1-carboxylic acid;
(1S,2R,5R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-5-hydroxy-1-methylcyclohexane-1-carboxylic acid;
(1S,2R,4R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-4-hydroxy-1-methylcyclohexane-1-carboxylic acid;
(2S,3R)-3-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)tetrahydro-2H-pyran-2-carboxylic acid;
(1R,2R,6S)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-6-hydroxy-1-methylcyclohexane-1-carboxylic acid;
(1R,2R,6R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-6-hydroxy-1-methylcyclohexane-1-carboxylic acid;
(1S,2S,3R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-3-hydroxy-1-methylcyclohexane-1-carboxylic acid;
(1S,2S,3S)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-3-hydroxy-1-methylcyclohexane-1-carboxylic acid;
5-(((S)-5-chloro-8-((5-(difluoromethyl)-1-methyl-1H-1,2,3-triazol-4-yl)methoxy)-7-fluoro-2-((1R,2S)-2-methyl-2-(1H-tetrazol-5-yl)cyclohexane-1-carbonyl)-1,2,3,4-tetrahydroisoquinolin-1-yl)methyl)-5-azaspiro[2.4]heptan-6-one;
(1S,2R)-2-((S)-5-chloro-8-((5-(difluoromethyl)-1-methyl-1H-1,2,3-triazol-4-yl)methoxy)-7-fluoro-1-((3-oxomorpholino)methyl)-1,2,3,4-tetrahydroisoquinoline-2-carbonyl)-1-methylcyclohexane-1-carboxylic acid;
(1S,2R)-2-((S)-5-chloro-8-((5-(difluoromethyl)-1-methyl-1H-1,2,3-triazol-4-yl)methoxy)-7-fluoro-1-(((R)-4-methyl-2-oxopyrrolidin-1-yl)methyl)-1,2,3,4-tetrahydroisoquinoline-2-carbonyl)-1-methylcyclohexane-1-carboxylic acid;
(1S,2R)-2-((S)-5-chloro-7-fluoro-1-((6-oxo-5-azaspiro[2.4]heptan-5-yl)methyl)-8-((4,5,6,7-tetrahydro-[1,2,3]triazolo[1,5-a]pyridin-3-yl)methoxy)-1,2,3,4-tetrahydroisoquinoline-2-carbonyl)-1-methylcyclopentane-1-carboxylic acid;
(1S,2R)-2-((S)-5-chloro-8-((4,5-dimethyl-4H-1,2,4-triazol-3-yl)methoxy)-7-fluoro-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-7-fluoro-8-((1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-5-chloro-7-fluoro-8-((5-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxylic acid;
(1S,2R)-2-((S)-1-((1,3-dioxoisoindolin-2-yl)methyl)-8-(4-(methylamino)-4-oxobutoxy)-1,2,3,4-tetrahydroisoquinoline-2-carbonyl)-N-methylcyclohexane-1-carboxamide;
N-(2-(((S)-1-((1,3-dioxoisoindolin-2-yl)methyl)-2-((1R,2S)-2-(ethylcarbamoyl)cyclohexane-1-carbonyl)-1,2,3,4-tetrahydroisoquinolin-8-yl)oxy)ethyl)-5-methylisoxazole-3-carboxamide;
(1S,2R)-2-((S)-1-(cyclopropanecarboxamidomethyl)-8-(((S)-1-(thiazole-5-carbonyl)pyrrolidin-3-yl)oxy)-1,2,3,4-tetrahydroisoquinoline-2-carbonyl)-N-methylcyclohexane-1-carboxamide;
(1S,2R)-2-((S)-8-((1H-pyrazol-5-yl)methoxy)-1-((1,3-dioxoisoindolin-2-yl)methyl)-1,2,3,4-tetrahydroisoquinoline-2-carbonyl)-N-methylcyclohexane-1-carboxamide;
(1S,2R)-2-((S)-1-(acetamidomethyl)-8-(((S)-1-(thiazole-5-carbonyl)pyrrolidin-3-yl)oxy)-1,2,3,4-tetrahydroisoquinoline-2-carbonyl)-N-methylcyclohexane-1-carboxamide;
(1S,2R)—N-methyl-2-((S)-1-((2-oxooxazolidin-3-yl)methyl)-8-(((S)-1-(thiazole-5-carbonyl)pyrrolidin-3-yl)oxy)-1,2,3,4-tetrahydroisoquinoline-2-carbonyl)cyclohexane-1-carboxamide;
4-(((S)-1-((1,3-dioxoisoindolin-2-yl)methyl)-2-((1R,2S)-2-(methylcarbamoyl)cyclohexane-1-carbonyl)-1,2,3,4-tetrahydroisoquinolin-8-yl)oxy)butanoic acid;
(R)-1-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)-N-methylpiperidine-2-carboxamide;
(S)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-N-methylpyrrolidine-1-carboxamide;
1-(((S)-5-chloro-2-((S)-1-(2,2-difluoroacetyl)piperidine-2-carbonyl)-8-((5-(difluoromethyl)-1-methyl-1H-1,2,3-triazol-4-yl)methoxy)-1,2,3,4-tetrahydroisoquinolin-1-yl)methyl)pyrrolidin-2-one;
(S)-6-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-N-methyl-5-azaspiro[2.4]heptane-5-carboxamide;
(1R,2S)-2-((R)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-N,1-dimethylcyclohexane-1-carboxamide;
(1S,2R)-2-((S)-1-((1,3-dioxoisoindolin-2-yl)methyl)-8-(4-oxo-4-(pyrrolidin-1-yl)butoxy)-1,2,3,4-tetrahydroisoquinoline-2-carbonyl)cyclohexane-1-carboxamide;
5-cyclopropyl-N-(2-(((S)-1-((1,3-dioxoisoindolin-2-yl)methyl)-2-((1R,2S)-2-(methylcarbamoyl)cyclohexane-1-carbonyl)-1,2,3,4-tetrahydroisoquinolin-8-yl)oxy)ethyl)isoxazole-3-carboxamide;
N-(2-(((S)-1-((1,3-dioxoisoindolin-2-yl)methyl)-2-((1R,2S)-2-(methylcarbamoyl)cyclohexane-1-carbonyl)-1,2,3,4-tetrahydroisoquinolin-8-yl)oxy)ethyl)-4,5-dimethylisoxazole-3-carboxamide;
(1S,2R)-2-((S)-8-(((S)-1-acetylpyrrolidin-3-yl)oxy)-1-((1,3-dioxoisoindolin-2-yl)methyl)-1,2,3,4-tetrahydroisoquinoline-2-carbonyl)-N-methylcyclohexane-1-carboxamide;
N-(2-(((S)-2-((1R,2S)-2-(cyclopropylcarbamoyl)cyclohexane-1-carbonyl)-1-((1,3-dioxoisoindolin-2-yl)methyl)-1,2,3,4-tetrahydroisoquinolin-8-yl)oxy)ethyl)-5-methylisoxazole-3-carboxamide;
N-(2-(((S)-1-((1,3-dioxoisoindolin-2-yl)methyl)-2-((1R,2S)-2-(3-hydroxyazetidine-1-carbonyl)cyclohexane-1-carbonyl)-1,2,3,4-tetrahydroisoquinolin-8-yl)oxy)ethyl)-5-methylisoxazole-3-carboxamide;
N-(2-(((S)-2-((1R,2S)-2-(cyclobutylcarbamoyl)cyclohexane-1-carbonyl)-1-((1,3-dioxoisoindolin-2-yl)methyl)-1,2,3,4-tetrahydroisoquinolin-8-yl)oxy)ethyl)-5-methylisoxazole-3-carboxamide;
5-cyclopropyl-N-(2-(((S)-1-((1,3-dioxoisoindolin-2-yl)methyl)-2-((1R,2S)-2-(methylcarbamoyl)cyclohexane-1-carbonyl)-1,2,3,4-tetrahydroisoquinolin-8-yl)oxy)ethyl)isoxazole-3-carboxamide;
N-(2-(((S)-1-((1,3-dioxoisoindolin-2-yl)methyl)-2-((1R,2S)-2-(methylcarbamoyl)cyclohexane-1-carbonyl)-1,2,3,4-tetrahydroisoquinolin-8-yl)oxy)ethyl)benzo[d]oxazole-2-carboxamide;
N-(2-(((S)-1-((1,3-dioxoisoindolin-2-yl)methyl)-2-((1R,2S)-2-(methylcarbamoyl)cyclohexane-1-carbonyl)-1,2,3,4-tetrahydroisoquinolin-8-yl)oxy)ethyl)benzo[d]isoxazole-3-carboxamide;
(1S,2R)-2-((S)-1-((1,3-dioxoisoindolin-2-yl)methyl)-8-(2-((5-methylisoxazole)-4-sulfonamido)ethoxy)-1,2,3,4-tetrahydroisoquinoline-2-carbonyl)-N-methylcyclohexane-1-carboxamide;
(1S,2R)-2-((S)-1-((1,3-dioxoisoindolin-2-yl)methyl)-8-(((S)-1-(pyrimidine-5-carbonyl)pyrrolidin-3-yl)oxy)-1,2,3,4-tetrahydroisoquinoline-2-carbonyl)-N-methylcyclohexane-1-carboxamide;
(1S,2R)-2-((S)-1-((1,3-dioxoisoindolin-2-yl)methyl)-8-(((S)-1-nicotinoylpyrrolidin-3-yl)oxy)-1,2,3,4-tetrahydroisoquinoline-2-carbonyl)-N-methylcyclohexane-1-carboxamide;
(1S,2R)-2-((S)-1-((1,3-dioxoisoindolin-2-yl)methyl)-8-(((S)-1-((5-methylisoxazol-4-yl)sulfonyl)pyrrolidin-3-yl)oxy)-1,2,3,4-tetrahydroisoquinoline-2-carbonyl)-N-methylcyclohexane-1-carboxamide;
(1S,2R)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)-N-methylcyclohexane-1-carboxamide;
N-(2-(((S)-1-((1,3-dioxoisoindolin-2-yl)methyl)-2-((1R,2S)-2-(methylcarbamoyl)cyclohexane-1-carbonyl)-1,2,3,4-tetrahydroisoquinolin-8-yl)oxy)ethyl)thiazole-2-carboxamide;
(1S,2R)-2-((S)-8-(((S)-1-(cyclopropanecarbonyl)pyrrolidin-3-yl)oxy)-1-((1,3-dioxoisoindolin-2-yl)methyl)-1,2,3,4-tetrahydroisoquinoline-2-carbonyl)-N-methylcyclohexane-1-carboxamide;
(1S,2R)-2-((S)-1-((1,3-dioxoisoindolin-2-yl)methyl)-8-(((S)-1-(5-methylisoxazole-3-carbonyl)pyrrolidin-3-yl)oxy)-1,2,3,4-tetrahydroisoquinoline-2-carbonyl)-N-methylcyclohexane-1-carboxamide;
(1S,2R)-2-((S)-1-((1,3-dioxoisoindolin-2-yl)methyl)-8-(oxazol-5-ylmethoxy)-1,2,3,4-tetrahydroisoquinoline-2-carbonyl)-N-methylcyclohexane-1-carboxamide;
(1S,2R)-2-((S)-1-((1,3-dioxoisoindolin-2-yl)methyl)-8-(((S)-1-(2-methylthiazole-5-carbonyl)pyrrolidin-3-yl)oxy)-1,2,3,4-tetrahydroisoquinoline-2-carbonyl)-N-methylcyclohexane-1-carboxamide;
(1S,2R)-2-((S)-8-(((S)-1-(2,4-dimethylthiazole-5-carbonyl)pyrrolidin-3-yl)oxy)-1-((1,3-dioxoisoindolin-2-yl)methyl)-1,2,3,4-tetrahydroisoquinoline-2-carbonyl)-N-methylcyclohexane-1-carboxamide;
(1S,2R)-2-((S)-1-((1,3-dioxoisoindolin-2-yl)methyl)-8-(((S)-1-(2-methylthiazole-4-carbonyl)pyrrolidin-3-yl)oxy)-1,2,3,4-tetrahydroisoquinoline-2-carbonyl)-N-methylcyclohexane-1-carboxamide;
(R)-4-((S)-1-((1,3-dioxoisoindolin-2-yl)methyl)-8-(((S)-1-(thiazole-5-carbonyl)pyrrolidin-3-yl)oxy)-3,4-dihydroisoquinolin-2(1H)-yl)-N,3-dimethyl-4-oxobutanamide:
(1S,2R)—N-methyl-2-((S)-1-((2-oxopyrrolidin-1-yl)methyl)-8-(((S)-1-(thiazole-5-carbonyl)pyrrolidin-3-yl)oxy)-1,2,3,4-tetrahydroisoquinoline-2-carbonyl)cyclohexane-1-carboxamide;
(1S,2R)-2-((S)-1-((1,3-dioxoisoindolin-2-yl)methyl)-8-(pyrazin-2-ylmethoxy)-1,2,3,4-tetrahydroisoquinoline-2-carbonyl)-N-methylcyclohexane-1-carboxamide;
(S)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)-N-methylpiperidine-1-carboxamide;
(1S,2R)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)-N-propylcyclohexane-1-carboxamide;
(1S,2R)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)-N-(2-methoxyethyl)cyclohexane-1-carboxamide;
(1S,2R)—N-(2,2-difluoroethyl)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)cyclohexane-1-carboxamide;
(1S,2R)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)-N-(2,2,2-trifluoroethyl)cyclohexane-1-carboxamide;
(1S,2R)—N-(cyanomethyl)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)cyclohexane-1-carboxamide;
(1S,2R)—N-(2,2-difluoro-3-hydroxypropyl)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)cyclohexane-1-carboxamide;
(1S,2R)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)-N-(2-hydroxyethyl)cyclohexane-1-carboxamide;
1-((S)-1-((S)-1-((1,3-dioxoisoindolin-2-yl)methyl)-8-(((S)-1-(thiazole-5-carbonyl)pyrrolidin-3-yl)oxy)-3,4-dihydroisoquinolin-2(1H)-yl)-1-oxopropan-2-yl)-3-methylurea;
(1S,2R)-2-((S)-8-(((S)-1-acetylpyrrolidin-3-yl)oxy)-5-chloro-1-((1,3-dioxoisoindolin-2-yl)methyl)-1,2,3,4-tetrahydroisoquinoline-2-carbonyl)-N-methylcyclohexane-1-carboxamide;
5-((S)-1-((1,3-dioxoisoindolin-2-yl)methyl)-8-(((S)-1-(thiazole-5-carbonyl)pyrrolidin-3-yl)oxy)-3,4-dihydroisoquinolin-2(1H)-yl)-N-methyl-5-oxopentanamide;
4-((S)-1-((1,3-dioxoisoindolin-2-yl)methyl)-8-(((S)-1-(thiazole-5-carbonyl)pyrrolidin-3-yl)oxy)-3,4-dihydroisoquinolin-2(1H)-yl)-N-methyl-4-oxobutanamide;
(1S,2R)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)-N-ethylcyclohexane-1-carboxamide;
(1S,2R)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)-N-(3-hydroxypropyl)cyclohexane-1-carboxamide;
(1S,2R)—N-methyl-2-((S)-1-(propionamidomethyl)-8-(((S)-1-(thiazole-5-carbonyl)pyrrolidin-3-yl)oxy)-1,2,3,4-tetrahydroisoquinoline-2-carbonyl)cyclohexane-1-carboxamide;
(1S,2R)-2-((S)-1-((1,3-dioxoisoindolin-2-yl)methyl)-8-((1-methyl-1H-pyrazol-4-yl)methoxy)-1,2,3,4-tetrahydroisoquinoline-2-carbonyl)-N-methylcyclohexane-1-carboxamide;
(1S,2R)-2-((S)-8-(((S)-1-acetylpyrrolidin-3-yl)oxy)-5-bromo-1-((1,3-dioxoisoindolin-2-yl)methyl)-1,2,3,4-tetrahydroisoquinoline-2-carbonyl)-N-methylcyclohexane-1-carboxamide;
methyl (S)-2-((S)-1-((1,3-dioxoisoindolin-2-yl)methyl)-8-(((S)-1-(thiazole-5-carbonyl)pyrrolidin-3-yl)oxy)-1,2,3,4-tetrahydroisoquinoline-2-carbonyl)pyrrolidine-1-carboxylate;
(1S,2R)-2-((S)-8-(benzo[d]isoxazol-3-ylmethoxy)-5-chloro-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-8-(benzo[d]isoxazol-3-ylmethoxy)-5-chloro-1-((2-oxopyrrolidin-1-yl)methyl)-1,2,3,4-tetrahydroisoquinoline-2-carbonyl)-N-ethylcyclohexane-1-carboxamide;
(1S,2R)-2-((S)-8-(benzo[d]isoxazol-3-ylmethoxy)-5-chloro-1-((2-oxopyrrolidin-1-yl)methyl)-1,2,3,4-tetrahydroisoquinoline-2-carbonyl)cyclohexane-1-carboxamide;
(1S,2R)-2-((S)-5-bromo-8-((5-isopropyl-1,2,4-oxadiazol-3-yl)methoxy)-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-5-bromo-8-((5-(difluoromethyl)-1-methyl-1H-1,2,3-triazol-4-yl)methoxy)-1-((1-oxoisoindolin-2-yl)methyl)-1,2,3,4-tetrahydroisoquinoline-2-carbonyl)-N-methylcyclohexane-1-carboxamide;
(1S,2R)-2-((S)-5-chloro-8-((5-isopropyl-1,2,4-oxadiazol-3-yl)methoxy)-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-N-methylcyclohexane-1-carboxamide;
(2R,3R)-4-((S)-5-chloro-8-((5-isopropyl-1,2,4-oxadiazol-3-yl)methoxy)-1-((2-oxopyrrolidin-1-yl)methyl)-3,4-dihydroisoquinolin-2(1H)-yl)-N,2,3-trimethyl-4-oxobutanamide;
(1S,2R)-2-((S)-5-chloro-1-((2-oxopyrrolidin-1-yl)methyl)-8-(pyridin-3-ylmethoxy)-1,2,3,4-tetrahydroisoquinoline-2-carbonyl)-N-methylcyclohexane-1-carboxamide;
(1S,2R)-2-((S)-5-chloro-1-((2-oxopyrrolidin-1-yl)methyl)-8-(pyridazin-3-ylmethoxy)-1,2,3,4-tetrahydroisoquinoline-2-carbonyl)-N-methylcyclohexane-1-carboxamide;
(1S,2R)-2-((S)-5-chloro-1-((2-oxopyrrolidin-1-yl)methyl)-8-(pyrazin-2-ylmethoxy)-1,2,3,4-tetrahydroisoquinoline-2-carbonyl)-N-methylcyclohexane-1-carboxamide;
(1S,2R)-2-((S)-5-chloro-1-((2-oxopyrrolidin-1-yl)methyl)-8-(pyridin-2-ylmethoxy)-1,2,3,4-tetrahydroisoquinoline-2-carbonyl)-N-methylcyclohexane-1-carboxamide;
(1S,2R)-2-((S)-5-chloro-1-((2-oxopyrrolidin-1-yl)methyl)-8-(pyrimidin-5-ylmethoxy)-1,2,3,4-tetrahydroisoquinoline-2-carbonyl)-N-methylcyclohexane-1-carboxamide;
2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-N-methylcyclopentane-1-carboxamide;
(1S,2R)-2-((S)-5-chloro-8-(2-(1-methyl-1H-1,2,3-triazol-4-yl)ethoxy)-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-5-chloro-8-((3-methyl-3H-imidazo[4,5-b]pyridin-6-yl)methoxy)-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-N,1-dimethylcyclohexane-1-carboxamide;
(1S,2R)-2-((S)-5-chloro-8-(isoxazolo[5,4-b]pyridin-3-ylmethoxy)-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-5-chloro-8-((3-methylisoxazolo[5,4-b]pyridin-6-yl)methoxy)-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-5-chloro-8-((1-methyl-1H-benzo[d][1,2,3]triazol-5-yl)methoxy)-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-8-([1,2,4]triazolo[4,3-a]pyridin-3-ylmethoxy)-5-chloro-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-5-chloro-8-((1-methyl-1H-1,2,3-triazol-4-yl)methoxy)-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-5-chloro-8-(imidazo[1,2-a]pyrimidin-2-ylmethoxy)-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-5-chloro-8-((5-(difluoromethyl)-1-methyl-1H-1,2,3-triazol-4-yl)methoxy)-1-((1-oxoisoindolin-2-yl)methyl)-1,2,3,4-tetrahydroisoquinoline-2-carbonyl)-N-methylcyclohexane-1-carboxamide;
(1S,2R)-2-((S)-8-([1,2,4]triazolo[4,3-a]pyridin-3-ylmethoxy)-5-chloro-1-((1-oxoisoindolin-2-yl)methyl)-1,2,3,4-tetrahydroisoquinoline-2-carbonyl)-N-methylcyclohexane-1-carboxamide;
(1S,2R)-2-((S)-8-(benzo[d]isoxazol-3-ylmethoxy)-5-chloro-1-((2-oxopyrrolidin-1-yl)methyl)-1,2,3,4-tetrahydroisoquinoline-2-carbonyl)-N-(2,2-difluoro-3-hydroxypropyl)cyclohexane-1-carboxamide;
(S)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-N-methylpiperidine-1-carboxamide;
(1S,2R)-2-((S)-5-chloro-8-((3-methyl-3H-imidazo[4,5-b]pyridin-5-yl)methoxy)-1-((2-oxopyrrolidin-1-yl)methyl)-1,2,3,4-tetrahydroisoquinoline-2-carbonyl)-N-methylcyclohexane-1-carboxamide;
(1S,2R)-2-((S)-5-chloro-8-((1-methyl-1H-indazol-3-yl)methoxy)-1-((2-oxopyrrolidin-1-yl)methyl)-1,2,3,4-tetrahydroisoquinoline-2-carbonyl)-N-methylcyclohexane-1-carboxamide;
3-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-N-methyltetrahydrofuran-2-carboxamide;
(1S,2R)-2-((S)-8-(benzo[d]isoxazol-3-ylmethoxy)-5-chloro-1-((2-oxopyrrolidin-1-yl)methyl)-1,2,3,4-tetrahydroisoquinoline-2-carbonyl)-N,1-dimethylcyclohexane-1-carboxamide;
(1S,2R)-2-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-N,1-dimethylcyclopentane-1-carboxamide;
(1S,2R)-2-((S)-5-chloro-8-((4,5-dimethyl-4H-1,2,4-triazol-3-yl)methoxy)-1-((2-oxopyrrolidin-1-yl)methyl)-1,2,3,4-tetrahydroisoquinoline-2-carbonyl)-N-methylcyclohexane-1-carboxamide;
(S)-3-((S)-5-chloro-8-((5-(difluoromethyl)-1-methyl-1H-1,2,3-triazol-4-yl)methoxy)-1-((2-oxopyrrolidin-1-yl)methyl)-1,2,3,4-tetrahydroisoquinoline-2-carbonyl)-N-methylmorpholine-4-carboxamide;
(S)-2-((S)-5-chloro-8-((1-methyl-1H-1,2,3-triazol-4-yl)methoxy)-1-((2-oxopyrrolidin-1-yl)methyl)-1,2,3,4-tetrahydroisoquinoline-2-carbonyl)-N-methylpyrrolidine-1-carboxamide;
(1S,2R)-2-((S)-5-chloro-8-((1,5-dimethyl-1H-1,2,3-triazol-4-yl)methoxy)-1-((2-oxopyrrolidin-1-yl)methyl)-1,2,3,4-tetrahydroisoquinoline-2-carbonyl)-N,1-dimethylcyclohexane-1-carboxamide;
(1S,2R)-2-((S)-5-chloro-8-((1-methyl-5-(trifluoromethyl)-1H-1,2,3-triazol-4-yl)methoxy)-1-((2-oxopyrrolidin-1-yl)methyl)-1,2,3,4-tetrahydroisoquinoline-2-carbonyl)-N,1-dimethylcyclohexane-1-carboxamide;
(S)-2-((S)-5-chloro-8-((1-methyl-1H-1,2,3-triazol-4-yl)methoxy)-1-((2-oxopyrrolidin-1-yl)methyl)-1,2,3,4-tetrahydroisoquinoline-2-carbonyl)-N-methylpiperidine-1-carboxamide;
(1S,2R)-2-((S)-5-chloro-8-((1-methyl-1H-1,2,3-triazol-4-yl)methoxy)-1-((2-oxopyrrolidin-1-yl)methyl)-1,2,3,4-tetrahydroisoquinoline-2-carbonyl)-N,1-dimethylcyclohexane-1-carboxamide;
(1S,2R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-N,1-dimethylcyclopentane-1-carboxamide;
(1S,2R)-2-((1S)-5-chloro-8-((5-(difluoromethyl)-1-methyl-1H-1,2,3-triazol-4-yl)methoxy)-1-((3-methyl-2-oxopyrrolidin-1-yl)methyl)-1,2,3,4-tetrahydroisoquinoline-2-carbonyl)-N,1-dimethylcyclopentane-1-carboxamide;
(1S,2R)-2-((1S)-5-chloro-8-((5-(difluoromethyl)-1-methyl-1H-1,2,3-triazol-4-yl)methoxy)-1-((3-methyl-2-oxopyrrolidin-1-yl)methyl)-1,2,3,4-tetrahydroisoquinoline-2-carbonyl)-N,1-dimethylcyclohexane-1-carboxamide;
(1S,2R)-2-((S)-5-chloro-8-((1-methyl-5-(trifluoromethyl)-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-N,1-dimethylcyclohexane-1-carboxamide;
(1S,2R)-2-((S)-5-chloro-8-((5-cyclopropyl-1-methyl-1H-1,2,3-triazol-4-yl)methoxy)-1-((2-oxopyrrolidin-1-yl)methyl)-1,2,3,4-tetrahydroisoquinoline-2-carbonyl)-N,1-dimethylcyclohexane-1-carboxamide;
(1S,2R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-1-methylcyclohexane-1-carboxamide;
(1S,2R)-2-((S)-5-chloro-8-((1,5-dimethyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-N,1-dimethylcyclohexane-1-carboxamide;
(1S,2R)-2-((S)-5-chloro-8-((1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-N,1-dimethylcyclohexane-1-carboxamide;
(1S,2R)-2-((S)-5-chloro-8-((5-cyclopropyl-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-N,1-dimethylcyclohexane-1-carboxamide;
(1S,2R)-2-((S)-5-chloro-8-((1-methyl-1H-1,2,3-triazol-4-yl)methoxy)-1-(((R)-4-methyl-2-oxopyrrolidin-1-yl)methyl)-1,2,3,4-tetrahydroisoquinoline-2-carbonyl)-N,1-dimethylcyclohexane-1-carboxamide;
(1S,2R)-2-((S)-5-chloro-8-((5-(methoxymethyl)-1-methyl-1H-1,2,3-triazol-4-yl)methoxy)-1-(((R)-4-methyl-2-oxopyrrolidin-1-yl)methyl)-1,2,3,4-tetrahydroisoquinoline-2-carbonyl)-N,1-dimethylcyclohexane-1-carboxamide;
(1S,2R)-2-((S)-5-chloro-8-((5-(difluoromethyl)-1-methyl-1H-1,2,3-triazol-4-yl)methoxy)-7-fluoro-1-((6-oxo-5-azaspiro[2.4]heptan-5-yl)methyl)-1,2,3,4-tetrahydroisoquinoline-2-carbonyl)-N,1-dimethylcyclohexane-1-carboxamide;
(1S,2R)-2-((S)-5-chloro-8-((5-(difluoromethyl)-1-methyl-1H-1,2,3-triazol-4-yl)methoxy)-7-fluoro-1-((6-oxo-5-azaspiro[2.4]heptan-5-yl)methyl)-1,2,3,4-tetrahydroisoquinoline-2-carbonyl)-1-methylcyclohexane-1-carboxamide;
(1S,2R)-2-((S)-5-chloro-8-((5-(difluoromethyl)-1-methyl-1H-1,2,3-triazol-4-yl)methoxy)-7-fluoro-1-((3-oxomorpholino)methyl)-1,2,3,4-tetrahydroisoquinoline-2-carbonyl)-N,1-dimethylcyclohexane-1-carboxamide;
(1S,2R)-2-((S)-5-chloro-8-((5-(difluoromethyl)-1-methyl-1H-1,2,3-triazol-4-yl)methoxy)-7-fluoro-1-((2-oxopyrrolidin-1-yl)methyl)-1,2,3,4-tetrahydroisoquinoline-2-carbonyl)-N,1-dimethylcyclohexane-1-carboxamide;
(1S,2R)-2-((S)-5-chloro-7-fluoro-8-((1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-N,1-dimethylcyclohexane-1-carboxamide;
(1S,2R)-2-((S)-5-chloro-7-fluoro-1-((6-oxo-5-azaspiro[2.4]heptan-5-yl)methyl)-8-((4,5,6,7-tetrahydro-[1,2,3]triazolo[1,5-a]pyridin-3-yl)methoxy)-1,2,3,4-tetrahydroisoquinoline-2-carbonyl)-1-methylcyclohexane-1-carboxamide;
(1S,2R)-2-((S)-5-chloro-8-((1,5-dimethyl-1H-1,2,3-triazol-4-yl)methoxy)-7-fluoro-1-((6-oxo-5-azaspiro[2.4]heptan-5-yl)methyl)-1,2,3,4-tetrahydroisoquinoline-2-carbonyl)-1-methylcyclohexane-1-carboxamide;
(1S,2R)-2-((S)-5-chloro-8-((5-cyclopropyl-1-methyl-1H-1,2,3-triazol-4-yl)methoxy)-1-(((R)-4-methyl-2-oxopyrrolidin-1-yl)methyl)-1,2,3,4-tetrahydroisoquinoline-2-carbonyl)-N,1-dimethylcyclohexane-1-carboxamide; and
(1S,2R)-2-((S)-5-chloro-8-((5-(difluoromethyl)-1-methyl-1H-1,2,3-triazol-4-yl)methoxy)-1-((6-oxo-5-azaspiro[2.4]heptan-5-yl)methyl)-1,2,3,4-tetrahydroisoquinoline-2-carbonyl)-N-methylcyclohexane-1-carboxamide.

19. The compound according to any one of claims 1 to 18, wherein L is a covalent bond or a bivalent, saturated or unsaturated, straight or branched hydrocarbon chain comprising 1-50 carbon atoms; wherein between 0 and 6 alkylene units of L are independently replaced by -Cy-, —O—, —N(R), —S—, —OC(O)—, —C(O)O—, —S(O)—, —S(O)₂—, —N(R)S(O)₂—, —S(O)₂N(R)—, —N(R)C(O)—, —C(O)N(R)—, —OC(O)N(R)—, 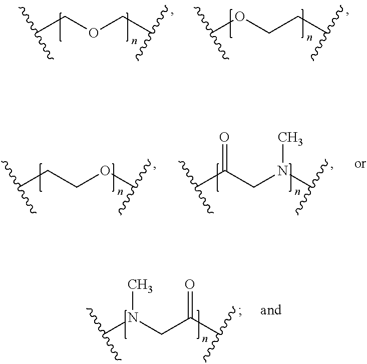
each -Cy- is independently an optionally substituted bivalent ring selected from:
i) phenylenyl,
ii) an 8-10 membered bicyclic arylenyl,
iii) a 4-7 membered saturated or partially unsaturated carbocyclenyl,
iv) a 4-7 membered saturated or partially unsaturated spiro carbocyclylenyl,
v) an 8-10 membered bicyclic saturated or partially unsaturated carbocyclylenyl,
vi) a 4-7 membered saturated or partially unsaturated heterocyclylenyl having 1-2 heteroatoms independently selected from nitrogen, oxygen, and sulfur,
vii) a 4-7 membered saturated or partially unsaturated spiro heterocyclylenyl having 1-2 heteroatoms independently selected from nitrogen, oxygen or sulfur,
viii) an 8-10 membered bicyclic saturated or partially unsaturated heterocyclylenyl having 1-2 heteroatoms independently selected from nitrogen, sulfur or oxygen,
ix) a 5-6 membered heteroarylenyl having 1-4 heteroatoms independently selected from nitrogen, oxygen and sulfur, and
x) an 8-10 membered bicyclic heteroarylenyl having 1-5 heteroatoms independently selected from nitrogen, oxygen or sulfur;
each n is independently 1-10; and
each R is independently hydrogen, or an optionally substituted group selected from C₁₋₆alkyl, phenyl, a 4-7 membered saturated or partially unsaturated heterocyclic having 1-2 heteroatoms independently selected from nitrogen, oxygen and sulfur, and a 5-6 membered heteroaryl ring having 1-4 heteroatoms independently selected from nitrogen, oxygen and sulfur.

20. The compound according to claim 19, wherein L is a bivalent, saturated or unsaturated, straight or branched hydrocarbon chain comprising 1-20 carbon atoms; wherein between 0 and 6 alkylene units of L are independently replaced by —O—, —N(R), —S—, —OC(O)—, —C(O)O—, —S(O)—, —S(O)₂—, —N(R)S(O)₂—, —S(O)₂N(R)—, —N(R)C(O)—, —C(O)N(R)—, —OC(O)N(R)—, a 4-7 membered saturated or partially unsaturated heterocyclylenyl having 1-2 heteroatoms independently selected from nitrogen, oxygen or sulfur, 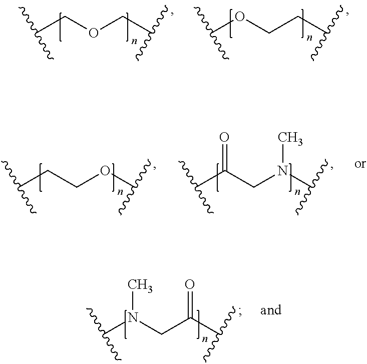
each n is independently 1-10; and each R is independently hydrogen or C₁₋₆alkyl.

21. The compound according to claim 20, wherein L is a bivalent, saturated, straight hydrocarbon chain comprising 5-15 carbon atoms; wherein between 2 and 6 alkylene units of L are independently replaced by —O—, —N(R), —S—, —OC(O)—, —C(O)O—, —S(O)—, —S(O)₂—, —N(R)S(O)₂—, —S(O)₂N(R)—, —N(R)C(O)—, —C(O)N(R)—, —OC(O)N(R)—, a 5-7 membered saturated heterocyclylenyl having 1-2 heteroatoms independently selected from nitrogen, oxygen, and sulfur, 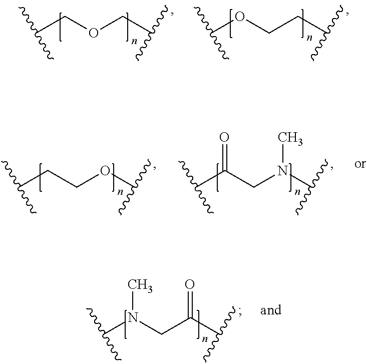
and
each n is independently 1-5; and each R is independently hydrogen, or C₁₋₃alkyl.

22. The compound according to claim 19, wherein L is selected from one of the following groups: 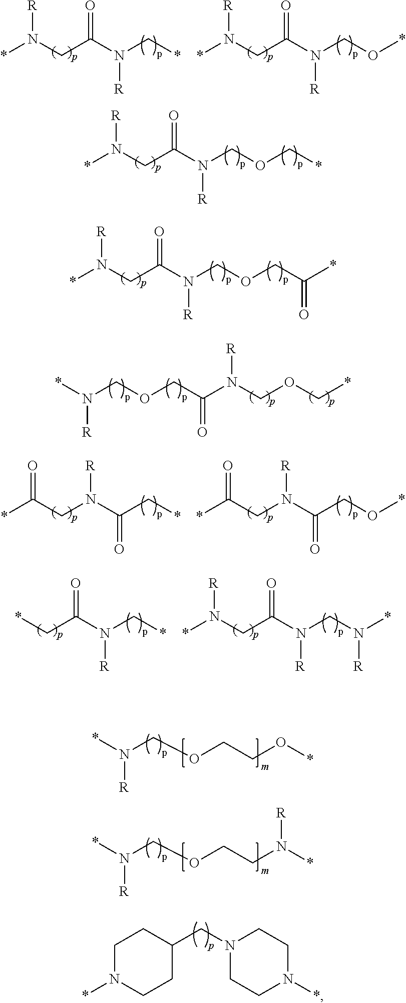
wherein * is the point of attachment to the KBG or the PBG; m is 0 to 4; each p is independently 1 to 4; and each R is independently hydrogen or C₁₋₃alkyl.

23. The compound according to any one of claims 1 to 22, wherein L is covalently attached to any one of the groups —R¹, —N(R⁴)(R⁵), or -L¹C(R⁶)(R⁷)L²C(R³)(R⁹)(R¹⁰) on the KBG.

24. The compound according to any one of claims 1 to 22, wherein L is covalently attached to the group —R¹.

25. The compound according to claim 24, wherein R¹ is a group selected from: 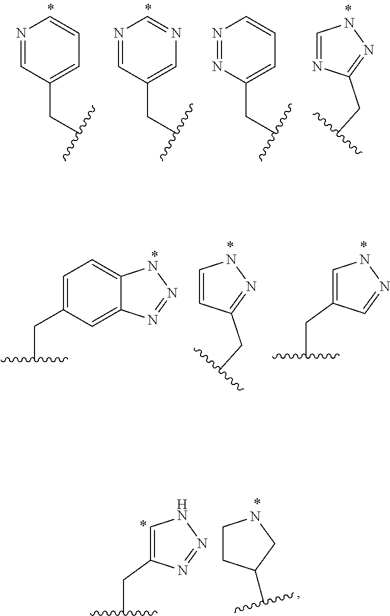
wherein [symbol] represents the point of attachment of the group to the oxygen atom of the rest of the KBG; each group is optionally substituted with one or more substituents independently selected from C₁₋₄alkyl, C₁₋₃haloalkyl, C₃₋₇cycloalkyl, C₁₋₄alkylene-R³⁰, halo, OH, C₁₋₃alkoxy, heterocyclyl and cyano; and wherein * represents the point of attachment of the linking group L.

26. The compound according to claim 23, wherein L is covalently attached to the group —N(R⁴)(R⁵) and replaces one of R⁴ or R⁵.

27. The compound according to claim 23, wherein L is covalently attached to the group —N(R⁴)(R⁵), and —N(R⁴)(R⁵) is selected from one of the following structures: 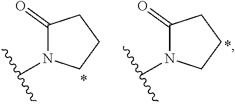
wherein [symbol] represents the point of attachment of the group to the rest of the KBG; each group is optionally substituted with one or more substituents independently selected from C₁₋₄alkyl, halo and OH; and wherein * represents the point of attachment of the linking group L.

28. The compound according to claim 23, wherein L is covalently attached to the group -L¹C(R⁶)(R⁷)L²C(R⁸)(R⁹)(R¹⁰) on the KBG, and -L¹C(R⁶)(R⁷)L²C(R⁸)(R⁹)(R¹⁰) is selected from one of the following structures: 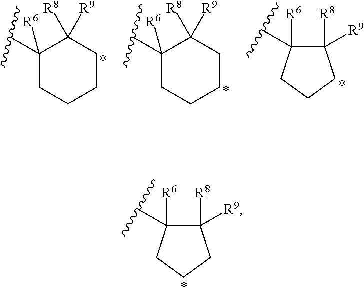
wherein [symbol] represents the point of attachment of the group to the rest of the KBG; each group is optionally substituted with one or more substituents independently selected from C₁₋₄alkyl, halo, OH, C₁₋₃alkoxy and deuterium; and wherein * represents the point of attachment of the linking group L.

29. The compound according to any one of claims 1 to 28, wherein the PBG is a protein binding group comprising a moiety having a binding affinity to a target protein of interest, wherein the target protein of interest is:
(i) a bromodomain and extra-terminal (BET) family protein (such as BRD3, BRD4 or BRD9);
(ii) an androgen receptor;
(iii) an estrogen receptor;
(iv) BCL-XL;
(v) IRAK4;
(vi) STAT3;
(vii) BTK; or
(viii) TRK.

30. The compound according to claim 29, wherein the target protein of interest is BRD4, and optionally wherein the moiety having a binding affinity is: 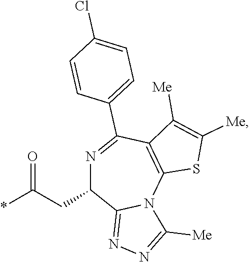 [SMILES: *C(=O)C[C@@H]1N=C(c2ccc(Cl)cc2)c2c(sc(C)c2C)-n2c(C)nnc21]
wherein * represents the point of attachment of the linking group L.

31. A pharmaceutical composition comprising a compound of Formula I according to any one of claims 1 to 30, or a pharmaceutically acceptable salt thereof, and one or more pharmaceutically acceptable excipients.

32. A compound of Formula I according to any one of claims 1 to 30, or a pharmaceutically acceptable salt thereof, or a pharmaceutical composition according to claim 31, for use in therapy.

33. A compound of Formula I according to any one of claims 1 to 30, or a pharmaceutically acceptable salt thereof, or a pharmaceutical composition according to claim 31, for use in the treatment of cancer or autoimmune diseases.

34. A compound of Formula I according to any one of claims 1 to 30, or a pharmaceutically acceptable salt thereof, or a pharmaceutical composition according to claim 31, for use in degrading proteins susceptible to degradation by KEAP ligase.

35. A method of degrading proteins susceptible to degradation by KEAP ligase in vitro, said method comprising:
a. administering an effective amount of a compound of Formula I according to any one of claims 1 to 30, or a pharmaceutically acceptable salt thereof; or
b. contacting a cell with an effective amount of a compound of Formula I according to any one of claims 1 to 30, or a pharmaceutically acceptable salt thereof.

36. A method of treating cancer or autoimmune diseases, comprising administering to a patient in need thereof a therapeutically effective amount of a compound of Formula I according to any one of claims 1 to 30, or a pharmaceutically acceptable salt thereof, or a pharmaceutical composition according to claim 31.
